(19) United States
(12) Patent Application Publication
(10) Pub. No.: US 2010/0254946 A1
(43) Pub. Date: Oct. 7, 2010

(54) MAINTENANCE OF HAIR GROWTH AND TREATMENT OF HAIR-LOSS

(75) Inventors: Regina Betz, Bonn (DE); Markus Nothen, Bonn (DE); Sandra Pasternack, Bonn (DE); Khalid Al Aboud, Makkah (SA)

Correspondence Address: MUETING, RAASCH & GEBHARDT, P.A., P.O. BOX 581336, MINNEAPOLIS, MN, 55458-1336, US

(73) Assignee: LIFE & BRAIN GMBH, Bonn (DE)

(21) Appl. No.: 12/680,108

(22) PCT Filed: Sep. 25, 2008
(86) PCT No.: PCT/EP2008/008170

§ 371 Date: Jun. 15, 2010

(30) Foreign Application Priority Data
Sep. 25, 2007 (EP) 07018871.9

(51) Int. Cl. A61K 35/12; C07H 21/04; C12N 15/74; C12N 1/21; C07K 14/00; A61K 31/7088; A61K 38/16; G01N 33/53; C12P 21/00; C07K 16/18; C07H 21/00; G01N 33/50; A61P 17/14

(52) U.S. Cl. 424/93.2; 536/23.5; 435/320.1; 435/252.3; 530/300; 514/44 R; 514/20.7; 435/7.21; 435/69.1; 530/387.1; 536/23.1; 436/86; 436/94

(57) ABSTRACT

The present invention relates to a nucleic acid molecule, encoding a polypeptide having P2Y5 receptor function wherein said nucleic acid molecule comprises: (a) a nucleic acid molecule encoding a polypeptide having the amino acid sequence of SEQ ID NO:2; (b) a nucleic acid molecule having the DNA sequence of SEQ ID NO:1; (c) a nucleic acid molecule having the sequence of SEQ ID NO:1, wherein each thymidine is replaced by uridine; (d) a nucleic acid molecule that hybridizes under stringent conditions to the complementary strand of a nucleic acid molecule of (a), (b) or (c); (e) a nucleic acid molecule encoding a polypeptide having at least 80% sequence identity to the polypeptide of (a); or (f) a nucleic acid molecule that is degenerate with respect to the nucleic acid molecule of (b), (c) or (d); for the diagnosis, treatment and/or prevention of hair-loss and the diagnosis of a predisposition for hair-loss. Furthermore, the invention relates to compositions and uses for the diagnosis, treatment and/or prevention of hair-loss as well as to methods of identifying compounds useful in the treatment of hair-loss. The invention also relates to nucleic acid molecules carrying mutations that are causative and/or indicative of hair-loss, diagnostic compositions, kits and methods for testing for the presence of the mutant nucleic acid molecule.

The present invention relates to a nucleic acid molecule, encoding a polypeptide having P2Y5 receptor function wherein said nucleic acid molecule comprises: (a) a nucleic acid molecule encoding a polypeptide having the amino acid sequence of SEQ ID NO:2; (b) a nucleic acid molecule having the DNA sequence of SEQ ID NO:1; (c) a nucleic acid molecule having the sequence of SEQ ID NO:1, wherein each thymidine is replaced by uridine; (d) a nucleic acid molecule that hybridizes under stringent conditions to the complementary strand of a nucleic acid molecule of (a), (b) or (c); (e) a nucleic acid molecules encoding a polypeptide having at least 80% sequence identity to the polypeptide of (a); or (f) a nucleic acid molecule that is degenerate with respect to the nucleic acid molecule of (b), (c) or (d); for the diagnosis, treatment and/or prevention of hair-loss and the diagnosis of a predisposition for hair-loss. Furthermore, the invention relates to compositions and uses for the diagnosis, treatment and/or prevention of hair-loss as well as to methods of identifying compounds useful in the treatment of hair-loss. The invention also relates to nucleic acid molecules carrying mutations that are causative and/or indicative of hair-loss, diagnostic compositions, kits and methods for testing for the presence of the mutant nucleic acid molecule.

Throughout this specification, several documents are cited. The disclosure content of these documents is herewith incorporated by reference (including all product descriptions and manufacturers instructions).

G protein-coupled receptors (GPCRs) constitute a major class of proteins responsible for transducing a signal within a cell. GPCRs share a common structural organization characterized by an amino terminal extracellular domain, seven hydrophobic alpha helices putatively constituting transmembrane domains, three extracellular loops, three intracellular loops, and a carboxy terminal intracellular domain.

Upon binding of a ligand to an extracellular portion of a GPCR, a signal is transduced within the cell that results in a change in a biological or physiological property of the cell. GPCRs, along with heterotrimeric, guanine nucleotide binding G proteins and effectors (intracellular enzymes and channels modulated by G proteins), are the components of a modular signaling system that connects the state of intracellular second messengers to extracellular inputs.

G protein-coupled receptor proteins are present on the cell surface of each functional cell and organ in the body, and they play important physiological roles as targets of the molecules that regulate their functions, e.g., hormones, neurotransmitters, physiologically active substances and the like. GPCR gene-products and its ligands are related to the occurrence of numerous metabolic and genetic disorders. Mechanistically, approximately 50-60% of all clinically relevant drugs act by modulating the functions of various GPCRs.

The P2Y5 receptor is an orphan G protein-coupled receptor and contains seven predicted hydrophobic transmembrane regions, a structural feature of G protein-coupled receptors. P2Y5 was originally reported to bind extracellular nucleotides as ligands (T. E. Webb et al., Biochem Biophys Res Commun 219, 105 (1996)), however, further experiments were not able to substantiate that these extracellular nucleotides are indeed P2Y5 ligands (Q. Li at al., Biochem Biophys Res Commun 236, 455 (1997); I. von Kügelgen, Pharmacol Ther 110, 415 (2006)). The gene encoding the P2Y5 receptor, P2RY5, has been published under the accession number uc001vcf.1 (database UCSC (University of California Santa Cruz), NCBI Build 36.1). It is localized in reverse orientation in intron 17 of the RB1 (retinoblastoma) gene (Herzog et al., Genome Research 6, 858 (1996)). The gene consists of only one coding exon encoding 344 amino acids. Seven 5′-untranslated regions (UTR) and one 3′-untranslated region were given in the database (UCSC).

Hair loss is a common occurrence in humans and has a variety of causes. The causes include purely genetic factors, as in androgenetic alopecia, multifactorial factors i.e. genetic as well as external factors, as in alopecia greata, or mainly external factors as in drug-induced alopecia. Hair loss causes significant psychological distress in the majority of those affected. Currently available therapies are unsatisfactory and there is a demand for novel, treatment strategies. A powerful approach to furthering our understanding of the pathophysiology of human hair loss is the identification of the genes underlying Mendelian isolated alopecias. Investigation of this type of hair loss offers the unique opportunity of identifying factors that are not only necessary for, but also specific to hair growth.

A number of genes have already been identified using this approach.

Ahmad at al. identified a human homolog of the murine hairless gene on chromosome 8p12 (W. Ahmad at al., Science 279, 720 (1998)). In individuals with alopecia universalis this gene was found to carry a missense mutation, which may affect the function of the gene, which encodes for a zinc finger transcription factor. Work by Levy-Nissenbaum et al. identified nonsense mutations in the gene encoding corneodesmosin (CDSN), a glycoprotein expressed in the epidermis and inner root sheath of hair follicles that act as a keratinocyte adhesion molecule (E. Levy-Nissenbaum et al., Nat Genet. 34, 151 (2003)). These nonsense mutations result in premature stop codons in CDSN in individuals suffering from hypotrichosis simplex of the scalp, an autosomal dominant form of isolated alopecia, Kljuic at al. identified another gene, the cadherin family member desmoglein 4, that carries mutations in individuals with autosomal recessive hypotrichosis (LAH) (A. Kljuic et al., Cell 113, 249 (2003)).

Recent work by Kazantseva et al. identified the lipase H (LIPH) gene as a further gene involved in human hair growth and scalp hair loss (A. Kazantseva et al., Science 314, 982 (2006)) while work by Miller et al. suggests a role of the vitamin D receptor gene (J. Miller et al., J Invest Dermatol 117, 612 (2001)).

Despite significant progress in this research field, the complex pathophysiology of human hair growth is far from completely understood, and a major breakthrough in therapy has yet to be achieved.

The technical problem underlying the present invention was the provision of means and methods useful in the diagnosis of hair-loss or a predisposition thereto.

The solution to this problem is achieved by providing the embodiments as characterized in the claims.

Accordingly, the present invention relates to a nucleic acid molecule, encoding a polypeptide having P2Y5 receptor function wherein said nucleic acid molecule comprises (a) a nucleic acid molecule encoding a polypeptide having the amino acid sequence of SEQ ID NO:2; (b) a nucleic acid molecule having the DNA sequence of SEQ ID NO:1; (c) a nucleic acid molecule having the sequence of SEQ ID NO:1, wherein each thymidine is replaced by uridine; (d) a nucleic acid molecule that hybridizes under stringent conditions to the complementary strand of a nucleic acid molecule of (a), (b) or (c); (e) a nucleic acid molecule encoding a polypeptide having at least 80% sequence identity to the polypeptide of (a); or (f) a nucleic acid molecule that is degenerate with respect to the nucleic acid molecule of (b), (c) or (d); for the diagnosis, treatment and/or prevention of hair-loss and the diagnosis of a predisposition for hair-loss.

Nucleic acid molecules, in accordance with the present invention, include DNA, such as cDNA or genomic DNA, RNA (e.g. mRNA), also in synthetic or semisynthetic form, further synthetic or semisynthetic derivatives of DNA or RNA (e.g. PNA or phosphorothioates) and mixed polymers, both sense and antisense strands. They may contain additional non-natural or derivatized nucleotide bases, as will be readily appreciated by those skilled in the art. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA) and locked nucleic acid (LNA) (see Braasch and Corey, Chem Biol 2001, 8: 1). LNA is an RNA derivative in which the ribose ring is constrained by a methylene linkage between the 2′-oxygen and the 4′-carbon. They may contain additional non-natural or derivative nucleotide bases, as will be readily appreciated by those skilled in the art. For the purposes of the present invention, a peptide nucleic acid (PNA) is a polyamide type of DNA analog and the monomeric units for the derivatives of adenine, guanine, thymine and cytosine are available commercially (Perceptive Biosystems). Certain components of DNA, such as phosphorus, phosphorus oxides, or deoxyribose derivatives, are not present in PNAs. As disclosed by Nielsen et al., Science 254:1497 (1991); and Egholm et al., Nature 365:666 (1993), PNAs bind specifically and tightly to complementary DNA strands and are not degraded by nucleases. In fact, PNA binds more strongly to DNA than DNA itself does. This is probably because there is no electrostatic repulsion between the two strands, and also the polyamide backbone is more flexible. Because of this, PNA/DNA duplexes bind under a wider range of stringency conditions than DNA/DNA duplexes, making it easier to perform multiplex hybridization. Smaller probes can be used than with DNA due to the strong binding. In addition, it is more likely that single base mismatches can be determined with PNA/DNA hybridization because a single mismatch in a PNA/DNA 15-mer lowers the melting point (T.sub.m) by 8°-20° C., vs. 4°-16° C. for the DNA/DNA 15-mer duplex. Also, the absence of charge groups in PNA means that hybridization can be done at low ionic strengths and reduce possible interference by salt during the analysis.

In a preferred embodiment the nucleic acid molecule is DNA.

The term “polypeptide”, which is interchangeably used herein with the term “protein”, in accordance with the present invention describes molecular chains of amino acids, including single chain proteins or their fragments, containing 30 or more amino acids. Preferably, these amino acid chains are linear. Polypeptides may further form multimers consisting of at least two identical or different molecules. The corresponding higher order structures of such multimers are correspondingly termed homo- or heterodimers, homo- or heterotrimers etc. Homodimers, timers etc. of fusion proteins giving rise or corresponding to polypeptides of the present invention also fall under the definition of the term “polypeptide” or “protein”. Those components of said fusion proteins, which are not P2Y5 protein sequences or fragments or variants thereof as defined herein above, include amino acid sequence which confer desired properties such as modified/enhanced stability, modified/enhanced solubility and/or the ability of targeting one or more specific cell types or could confer a different biological activity. For example, fusion proteins with antibodies specific for cell surface markers or with antigen-recognizing fragments of said antibodies are envisaged. Furthermore, peptidomimetics of such proteins/polypeptides where amino acid(s) and/or peptide bond(s) have been replaced by functional analogues are also encompassed by the invention. Such functional analogues include all known amino acids other than the 20 gene-encoded amino acids, such as selenocysteine. The terms “polypeptide” and “protein” also refer to naturally modified polypeptides/proteins where the modification is effected e.g. by glycosylation, acetylation, phosphorylation and similar modifications which are well known in the art.

It is also well known that polypeptides are not always entirely linear. For instance, polypeptides may be branched as a result of ubiquitination, and they may be circular, with or without branching, generally as a result of post-translation events, including natural processing events and events brought about by human manipulation which do not occur naturally. Circular, branched and branched circular polypeptides may be synthesized by non-translational natural processes and by synthetic methods.

The modifications can be a function of how the protein is made. For recombinant polypeptides, for example, the modifications will be determined by the host cells posttranslational modification capacity and the modification signals in the polypeptide amino acid sequence. Accordingly, when glycosylation is desired, a polypeptide should be expressed in a glycosylating host, generally an eukaryotic cell, for example Cos7, HELA or others. The same type of modification may be present in the same or varying degree at several sites in a given polypeptide. Also, a given polypeptide may contain more than one type of modification.

The term “P2Y5 receptor” in accordance with the present invention refers to the orphan G protein-coupled receptor (GPCR) P2Y5.

The coding sequence of the P2RY5 gene is shown in SEQ ID NO:1 and the deduced 344 amino acid sequence is shown in SEQ ID NO:2. The genomic sequence of the P2RY5 gene is represented in SEQ ID NO: 4 while the sequence corresponding the P2RY5 mRNA (including 5′ and 3′ UTRs) is represented in SEQ ID NO:3. It is predicted that amino acids 1 to about 19 constitute the amino terminal extracellular domain, amino acids about 20-292 constitute the region spanning the transmembrane domain, and amino acids about 293-344 constitute the carboxy terminal intracellular domain. The transmembrane domain contains seven transmembrane segments, three extracellular loops and three intracellular loops. As used herein, the term “transmembrane segment” refers to a structural amino acid motif which includes a hydrophobic helix that spans the plasma membrane. The transmembrane segments are found from about amino acid 20 to about amino acid 46, from about amino acid 56 to about amino acid 79, from about amino acid 93 to about amino acid 113, from about amino acid 134 to about amino acid 154, from about amino acid 182 to about amino acid 209, from about amino acid 228 to about amino acid 253, and from about amino acid 273 to about amino acid 292. Within the region spanning the entire transmembrane domain are three intracellular and three extracellular loops. The three intracellular loops are found from about amino acid 47 to about amino acid 55, from about amino acid 114 to about amino acid 133, and from about amino acid 210 to about amino acid 227. The three extracellular loops are found at from about amino acid 80 to about amino acid 92, from about amino acid 155 to about amino acid 181, and from about amino acid 254 to about amino acid 272.

The nucleic acid molecules of any of (d) to (f) include nucleic acid molecules encoding polypeptides comprising or consisting of fragments of the amino acid sequence set forth in SEQ ID NO: 2. It is well known in the art that functional polypeptides may be cleaved to yield fragments with unaltered or substantially unaltered function. Such cleavage may include the removal of a given number of N- and/or C-terminal amino acids. Additionally or alternatively, a number of internal (non-terminal) amino acids may be removed, provided the obtained polypeptide has P2Y5 receptor activity. Said number of amino acids to be removed from the termini and/or internal regions may be one, two, three, four, five, six, seven, eight, nine, ten, 15, 20, 25, 30, 40, 50 or more than 50. Any other number between one and 50 is also deliberately envisaged. In particular, removals of amino acids which preserve sequence and boundaries of any conserved functional domain(s) or subsequences in the sequence of SEQ ID NO: 2 are particularly envisaged. Means and methods for determining such domains are well known in the art and include experimental and bioinformatic means. Experimental means include the systematic generation of deletion mutants and their assessment in assays for P2Y5 receptor activity known in the art and as described in the Examples enclosed herewith. Bioinformatic means include database searches. Suitable databases include protein sequence databases. In this case a multiple sequence alignment of significant hits is indicative of domain boundaries; wherein the domain(s) is/are comprised of the/those subsequences exhibiting an elevated level of sequence conservation as compared to the remainder of the sequence. Further suitable databases include databases of statistical models of conserved protein domains such as Pfam maintained by the Sanger Institute, UK (www.sanger.ac.uk/Software/Pfam).

Nucleic acid molecules of the invention comprising a nucleic acid molecule having the DNA sequence of SEQ ID NO:1 include, but are not limited to, nucleic acid molecules having the sequence of SEQ ID NO:1, nucleic acid molecules having the sequence of SEQ ID NO:1 and additional coding sequences, such as leader or secretory sequence as well as nucleic acid molecules having the sequence of SEQ ID NO:1 and additional non-coding sequences, e.g. 5′ and 3′ untranslated sequences (UTR) that are transcribed but not translated, such as for example SEQ ID NO:3. Such additional sequence may play a role in transcription, mRNA processing (including splicing and polyadenylation signals), ribosome binding and stability of mRNA. In addition, the nucleic acid molecule may be the entire genomic DNA representing the P2RY5 gene (as represented in SEQ ID NO:4). Furthermore, the nucleic acid molecule may comprise the sequence of SEQ ID NO:1 fused to marker sequences encoding, for example, a peptide that facilitates purification of the encoded polypeptide, such as a V5- or poly-His-tag.

The term “hybridizes/hybridizing” as used herein refers to a pairing of a polynucleotide to a (partially) complementary strand of this polynucleotide which thereby form a hybrid.

It is well known in the art how to perform hybridization experiments with nucleic acid molecules. Correspondingly, the person skilled in the art knows what hybridization conditions she/he has to use to allow for a successful hybridization in accordance with item (i)(c), above. The establishment of suitable hybridization conditions is referred to in standard text books such as Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989), or Higgins and Hames (Eds.) “Nucleic acid hybridization, a practical approach” IRL Press Oxford, Washington D.C., (1985).

“Stringent conditions” refers to hybridization conditions under which the polynucleotides that are capable of hybridizing to the polynucleotides of the invention or parts thereof hybridize to these target sequences to a detectably greater degree than to other sequences (e.g., at least 2-fold over background). Stringent conditions are sequence-dependent and will be different in different circumstances. By controlling the stringency of the hybridization and/or washing conditions, target sequences that have at least 90% sequence identity, more preferably 95%, such as 98% and more preferred 100% sequence identity to the probe can be identified (highly stringent hybridization conditions). Alternatively, stringency conditions can be adjusted to allow a higher degree of mismatching in sequences (low stringency conditions of hybridization). Such highly stringent and low stringent conditions for hybridization are well known to the person skilled in the art. For example, highly stringent conditions for hybridization comprise, e.g. an overnight incubation at 65° C. in 4×SSC (600 mM NaCl, 60 mM sodium citrate) followed by washing at 65° C. in 0.1×SSC for one hour. Alternatively, highly stringent hybridization conditions can comprise: an overnight incubation at 42° C. in a solution comprising 50% formamide, 5×SSC (750 mM NaCl, 75 mM sodium citrate), 50 mM sodium phosphate (pH 7.6), 5×Denhardt's solution, 10% dextran sulphate, and 20 μg/ml denatured, sheared salmon sperm DNA, followed by washing in e.g. 0.1-0.5×SSC at about 55-65° C. for about 5 to 20 min. Also contemplated are nucleic acid molecules that hybridize to the polynucleotides of the invention at lower stringency hybridization conditions (“low stringency conditions for hybridization”). Changes in the stringency of hybridization are primarily accomplished through the manipulation of formamide concentration (lower percentages of formamide result in lowered stringency), salt conditions, or temperature. For example, lower stringency conditions include an overnight incubation at 50° C. in 4×SSC or an overnight incubation at 37° C. in a solution comprising 6×SSPE (20×SSPE=3M NaCl; 0.2M NaH₂PO₄; 0.02M EDTA, pH 7.4), 0.5% SDS, 30% formamide, 100 mg/ml salmon sperm blocking DNA; followed by washes at 50° C. with 1×SSPE, 0.1% SDS. In addition, to achieve an even lower stringency, washes performed following stringent hybridization can be done at higher salt concentrations (e.g. 5×SSC). It is of note that variations in the above conditions may be accomplished through the inclusion and/or substitution of alternate blocking reagents. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. Such modifications can generally be effected by the skilled person without further ado. The embodiment recited herein above preferably refers to highly stringent conditions. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed).

As stated herein above, preferred in accordance with the present invention are nucleic acid molecules which are capable of hybridizing to the nucleic acid molecules of the invention or parts thereof, under (highly) stringent hybridization conditions, i.e. which do not cross hybridize to nucleic acid molecules unrelated in nucleotide sequence. In accordance with item (d), above, nucleic acid molecules related but not identical in sequence with the nucleic acid molecules of items (a), (b) or (c) are also encompassed by the invention. In addition, the invention comprises according to item (d) fragments of the nucleic acid molecule of (a), (b) or (c). For all embodiments falling under item (d), it is essential in accordance with this embodiment, that the polypeptide encoded by this nucleic acid molecule retains or essentially retains P2Y5 receptor function.

In accordance with the present invention, P2Y5 receptor function is essentially retained, if at least 60% of the biological activity of the P2Y5 receptor activity are retained. Preferably, at least 75% or at least 80% of the P2Y5 receptor activity are retained. More preferred is that at least 90% such as at least 95%, even more preferred at least 98% such as at least 99% of the biological activity of the P2Y5 receptor are retained. Most preferred is that the biological activity is fully, i.e. to 100%, retained. Also in accordance with the invention are polypeptides having increased biological activity compared to P2Y5 receptor, i.e. more than 100% enzyme activity. Methods of assessing biological activity of a polypeptide are well known to the person skilled in the art and include without being limiting measuring receptor mediated changes in cAMP levels. Methods for determining cAMP levels are well known to the skilled person and are described, for example in George et al., Journal of Biomolecular Screening 2, 235 (1997).

In accordance with the present invention the nucleic acid molecule can also encode a polypeptide having at least 80% sequence identity to the polypeptide of (a). More preferably, the nucleic acid molecule encodes a polypeptide that has at least 85%, even more preferably 90% sequence identity to the polypeptide of (a). Even more preferably the nucleic acid molecule can encode a polypeptide that has at least 95 sequence identity to the polypeptide of (a) and most preferably 98% sequence identity to the polypeptide of (a).

In accordance with the present invention, the term “% sequence identity” describes the number of matches (“hits”) of identical nucleotides/amino acids of two or more aligned nucleic acid or amino acid sequences as compared to the number of nucleotides or amino acid residues making up the overall length of the nucleic acid or amino acid sequences (or the overall compared part thereof). In other terms, using an alignment, for two or more sequences or subsequences the percentage of amino acid residues or nucleotides that are the same (e.g., 80% or 85% identity) may be determined, when the (sub)sequences are compared and aligned for maximum correspondence over a window of comparison, or over a designated region as measured using a sequence comparison algorithm as known in the art, or when manually aligned and visually inspected. This definition also applies to the complement of a test sequence. Preferred nucleic acid molecules/polypeptides in accordance with the invention are those where the described identity exists over a region that is at least about 15 to 25 amino acids or nucleotides in length, more preferably, over a region that is at least about 50 to 100 amino acids or nucleotides in length. More preferred nucleic acid molecules/polypeptides in accordance with the present invention are those having the described sequence identity over the entire length of the nucleic acid molecule or polypeptide. Those having skill in the art will know how to determine percent sequence identity between/among sequences using, for example, algorithms such as those based on CLUSTALW computer program (Thompson Nucl. Acids Res. 2 (1994), 4673-4680) or FASTA (Pearson and Lipman, Proc. Natl. Acad. Sci., 1988, 85; 2444), as known in the art.

Although the FASTA algorithm typically does not consider internal non-matching deletions or additions in sequences, i.e., gaps, in its calculation, this can be corrected manually to avoid an overestimation of the % sequence identity. CLUSTALW, however, does take sequence gaps into account in its identity calculations. Also available to those having skill in this art are the BLAST and BLAST 2.0 algorithms (Altschul, Nucl. Acids Res., 1977, 25:3389). The BLASTN program for nucleic acid sequences uses as default a word length (W) of 11, an expectation (E) of 10, M=5, N=4, and a comparison of both strands. For amino acid sequences, the BLASTP program uses as default a word length (W) of 3, and an expectation (E) of 10. The BLOSUM62 scoring matrix (Henikoff, Proc. Natl. Acad. Sci., 1989, 89:10915) uses alignments (B) of 50, expectation (E) of 10, M=5, N=4, and a comparison of both strands. All those programs may be used for the purposes of the present invention. Accordingly, all the nucleic acid molecules having the prescribed function and further having a sequence identity of at least 80% as determined with any of the above recited or further programmes available to the skilled person fall under the scope of the invention.

In accordance with the present invention the nucleic acid molecule encoding a polypeptide having P2Y5 receptor function may be degenerate with respect to the nucleic acid molecule of item (b), (c) or (d). When used in accordance with the present invention the term being “degenerate” means that due to the redundancy of the genetic code different nucleotide sequences code for the same amino acid.

The term “hair-loss” in accordance with the present invention and as used throughout the description relates to and includes all hair-loss conditions including, for example, androgenetic alopecia, alopecia greata, monogenic and syndromal forms of alopecia. Also included within the term “hair-loss” are all forms of hair-loss induced by, for example, drugs (chemotherapy) or mechanic stimuli.

Androgenetic alopecia, the most common form of hair-loss, is triggered by elevated androgene receptor level and by elevated dihydrotestosterone (DHT) level, a sex hormone, that can adversely affect the hair on the scalp. However, the mechanism by which DHT accomplishes this is not yet understood.

Alopecia greata (AA) is a common dermatologic disease with a lifetime prevalence of 1-2%, which causes round, patchy hair loss on the scalp. A classification in three subtypes is established referring to the amount of hair loss, ranging from patchy AA (one or more circumscribed patches of hair loss) to alopecia totalis (AT, 100% loss of scalp hair without loss of body hair) and alopecia universalis (AU, 100% loss of both scalp and body hair). The etiopathogenesis of AA is incompletely understood, but AA is thought to be a tissue-specific autoimmune disease directed against the hair follicle. Induced hair-loss refers to exogenic factors, as for example drugs (chemotherapy) or, mechanic stimuli.

Monogenic forms of hair-loss are characterised by a single gene being responsible for the pathogenic cause of hair loss. Examples include, but are not limited to, Hypotrichosis simplex of the scalp (HSS), hypotrichosis simplex, generalised form (HSG), alopecia universalis congenitalis, papular atrichia, hypotrichosis Marie Unna and monilethrix.

The present inventors surprisingly found that the orphan G protein coupled receptor P2Y5 mediates signalling pathways that are crucial for the maintenance of hair growth. Dysregulation of the P2Y5 receptor, on the other hand, was shown to be associated with diseases such as hair-loss. To the applicants best knowledge, these are the first data that implicate a G protein-coupled receptor as being essential for and specific to the maintenance of human hair growth. Due to these findings it is now possible to develop new diagnostic tools to predict an individuals predispositions for diseases mediated by the P2Y5 receptor as well as diagnostic and therapeutic approaches for individuals affected by diseases mediated by the P2Y5 receptor, in particular hair-loss.

The technical teaching of the present invention has been confirmed by genetical and molecular biological data published by Shimomura Y. et al. 2008, (Nat. Genet.; 40(3):335-9) as well as by Azeem Z, et al., 2008 (Hum Genet.; 123(5):515-9). In addition, it has been found by the inventors that the bioactive lipid oleoyl-L-alpha-lysophosphatidic acid (LPA) is a ligand for the P2Y5 receptor (Pasternack S M et al., 2008, Nat. Genetics 40(3):329-34). Using this ligand in biochemical assays it has been confirmed that the P2Y5 mutant p.Lys125AsnfsX37 is not capable of activating a CRE luciferase reporter gene, thus indicating a loss of function of the receptor due to the mutation. Said ligand had previously been shown to stimulate hair growth in vivo in a mouse model, thus further corroborating the essential function of the P2Y5 receptor in hair growth.

Experimentally, using a genome-wide linkage-analysis, the present inventors identified a genetic defect underlying an autosomal-recessive mode of inherited hypotrichosis simplex, in a Saudi-Arabian family. After sequencing of various genes in the candidate region, a premature stop-codon in the G protein-coupled receptor P2Y5 was detected. When screening other hypotrichosis cases it was found that a different P2RY5-Mutation in two further HSG-families from Saudi-Arabia, enclosing one and two affected individuals respectively, also resulting in a truncated, non-functional protein. A number of diverse functional studies, such as expression-profiling in humans (cDNA panels (BD), multiple tissue expression human array3 (BD)), 5′RACE (Rapid amplification of 5′ complementary DNA ends) and Northern Blotting (mouse RNA), confirmed these results. Furthermore, the proteins were analyzed by Western blot, protein truncation tests (The TNT® 17 Quick Coupled Transcription/Translation System) and immunofluorescence experiments. In preferred embodiments of the invention, the hair-loss is selected from the group consisting of hypotrichosis simplex of the scalp (HSS), hypotrichosis simplex, generalised form (HSG), alopecia universalis congenitalis, papular atrichia, hypotrichosis Marie Unna and monilethrix.

Hypotrichosis simplex (HS; MIM 146520 and MIM 605389) is a rare form of non-syndromic alopecia that affects men and women equally. In most families the disorder is inherited as an autosomal dominant trait, although autosomal recessive inheritance has also been observed. The extent of scalp and body hair involvement is variable. Some families experience hypotrichosis that is confined to the scalp (hypotrichosis simplex of the scalp, HSS) while others experience loss of body hair in addition to scalp hair loss (hypotrichosis simplex, generalized form, HSG). The hair loss is diffuse and usually begins in early childhood and progresses until adulthood. Hypotrichosis from birth has also been reported. In hypotrichosis, the hair shaft characteristically shows no gross abnormality.

In a preferred embodiment, the nucleic acid molecule of the invention is contained in a vector.

The term “vector” in accordance with the present invention refers to a vector that is a plasmid, cosmid, virus, bacteriophage or another vector. Such vectors may be used e.g. in genetic engineering. Incorporation of the nucleic acid into a vector offers the possibility of introducing the nucleic acid molecule efficiently into the cells and preferably the DNA of a recipient. The recipient may be a single cell such as a cell from a cell line. Such a measure renders it possible to express, when expression vectors are chosen, the respective nucleic acid molecule in the recipient. Thus, incorporation of the nucleic acid molecule into an expression vector opens up the way to a permanently elevated level of the encoded protein in any cell or a subset of selected cells of the recipient. In a preferred embodiment, the recipient is a mammal. In a more preferred embodiment, the mammal is a human.

The nucleic acid molecule may be inserted into several commercially available vectors. Non-limiting examples include vectors compatible with an expression in mammalian cells like pREP (Invitrogen), pcDNA3 (Invitrogen), pCEP4 (Invitrogen), pMC1neo (Stratagene), pXT1 (Stratagene), pSG5 (Stratagene), EBO-pSV2neo, pBPV-1, pdBPVMMTneo, pRSVgpt, pRSVneo, pSV2-dhfr, pIZD35, pLXIN, pSIR (Clontech), pIRES-EGFP (Clontech), pEAK-10 (Edge Biosystems) pTriEx-Hygro (Novagen), pCINeo (Promega), Okayama-Berg cDNA expression vector pcDV1 (Pharmacia), pRc/CMV, pcDNA1, pSPORT1 (GIBCO BRL), pGEMHE (Promega), pSVL and pMSG (Pharmacia, Uppsala, Sweden), pRSVcat (ATCC 37152), pSV2dhfr (ATCC 37146), pBC12MI (ATCC 67109) or pcDNA3.1 (Invitrogen), preferably pcDNA3.1/V5/His or pcDNA5/FRT/TO (Invitrogen).

The nucleic acid molecule referred to above may also be inserted into vectors such that a translational fusion with another nucleic acid molecule is generated. The vectors may also contain an additional expressible polynucleotide coding for one or more chaperones to facilitate correct protein folding.

For vector modification techniques, see Sambrook and Russel (“Molecular Cloning, A Laboratory Manual”, Cold Spring Harbor Laboratory, N.Y. (2001)). Generally, vectors can contain one or more origin of replication (ori) and inheritance systems for cloning or expression, one or more markers for selection in the host, e.g., antibiotic resistance, and one or more expression cassettes.

The coding sequences inserted in the vector can e.g. be synthesized by standard methods, or isolated from natural sources. Ligation of the coding sequences to transcriptional regulatory elements and/or to other amino acid encoding sequences can be carried out using established methods. Transcriptional regulatory elements (parts of an expression cassette) ensuring expression in eukaryotic cells are well known to those skilled in the art. These elements comprise regulatory sequences ensuring the initiation of the transcription (a g. translation initiation codon, promoters, enhancers, and/or insulators), internal ribosomal entry sites (IRES) (Owens, Proc. Natl. Acad. Sci. USA 2001, 98: 1471) and optionally poly-A signals ensuring termination of transcription and stabilization of the transcript. Additional regulatory elements may include transcriptional as well as translational enhancers, and/or naturally-associated or heterologous promoter regions. Preferably, the nucleic acid molecule is operatively linked to such expression control sequences allowing expression in eukaryotic cells. The vector may further comprise nucleotide sequences encoding secretion signals as further regulatory elements. Such sequences are well known to the person skilled in the art. Furthermore, depending on the expression system used, leader sequences capable of directing the expressed polypeptide to a cellular compartment may be added to the coding sequence of the polynucleotide of the invention. Such leader sequences are well known in the art.

Possible examples for regulatory elements ensuring the initiation of transcription comprise the cytomegalovirus (CMV) promoter, SV40-promoter, RSV-promoter (Rous sarcome virus), the lacZ promoter, the gai10 promoter, human elongation factor 1a-promoter, CMV enhancer, CaM-kinase promoter, the Autographa californica multiple nuclear polyhedrosis virus (AcMNPV) polyhedral promoter or the SV40-enhancer. Examples for further regulatory elements in prokaryotes and eukaryotic cells comprise transcription termination signals, such as SV40-poly-A site or the tk-poly-A site or the SV40, lacZ and AcMNPV polyhedral polyadenylation signals, downstream of the polynucleotide. Moreover, elements such as origin of replication, drug resistance genes, regulators (as part of an inducible promoter) may also be included. Additional elements might include enhancers, Kozak sequences and intervening sequences flanked by donor and acceptor sites for RNA splicing. Highly efficient transcription can be achieved with the early and late promoters from SV40, the long terminal repeats (LTRs) from retroviruses, e.g., RSV, HTLVI, HIVI, and the early promoter of the cytomegalovirus (CMV). However, cellular elements can also be used (e.g., the human actin promoter).

The co-transfection with a selectable marker such as dihydrofolate reductase (dhfr), gpt, neomycin, hygromycin or G418 allows the identification and isolation of the transfected cells. The transfected nucleic acid can also be amplified to express large amounts of the encoded (poly)peptide. The DHFR marker is useful to develop cell lines that carry several hundred or even several thousand copies of the gene of interest. Another useful selection marker is the enzyme glutamine synthase (GS) (Murphy et al., Biochem J. 1991, 227:277; Bebbington et al., Bio/Technology 1992, 10:169). Using these markers, the mammalian cells are grown in selective medium and the cells with the highest resistance are selected. As indicated above, the expression vectors will preferably include at least one selectable marker.

The nucleic acid molecules as described herein may be designed for direct introduction or for introduction via liposomes, phage vectors or viral vectors (e.g. adenoviral, retroviral) into the cell. Additionally, baculoviral systems, or systems based on vaccinia virus or Semliki Forest virus can be used as eukaryotic expression system for the nucleic acid molecules of the invention.

In a further preferred embodiment, the nucleic acid molecule of the invention, which is contained in a vector, is contained in a host cell.

The term “host cell” in accordance with the present invention refers to a host cell that may be produced by introducing said vector into a host cell, which upon its presence mediates the expression of the nucleic acid molecule of the invention.

The host cell may be any prokaryote or eukaryotic cell. Suitable prokaryotes/bacteria are those generally used for cloning like E. coli (e.g., E coli strains BL21(DE3), HB101, DH5α, XL1 Blue, Y1090 and JM101), Salmonella typhimurium, Serratia marcescens, Pseudomonas putida, Pseudomonas fluorescens, Streptomyces lividans, Lactococcus lactis, Mycobacterium smegmatis or Bacillus subtilis. A suitable eukaryotic host cell may be an animal cell such as a mammalian cell, an amphibian cell, a fish cell, an insect cell such as Drosophila S2 and Spodoptera Sf9 cells, a fungal cell or a plant cell. Mammalian host cells that could be used include, human Hela, HEK293, H9, Bowes melanoma cells and Jurkat cells, mouse NIH3T3 and C127 cells, Cos 1, Cos 7 and CV1, quail QC1-3 cells, mouse L cells and Chinese hamster ovary (CHO) cells. Also within the scope of the present invention are primary mammalian cells such as the immortalised keratinocyte cell line HaCaT, primary keratinocytes, primary hair follicle cells or mouse embryonic fibroblasts (MEF). Appropriate culture mediums and conditions for the above-described host cells are well known in the art.

The present invention also relates to a polypeptide encoded by the nucleic acid molecule described above for the treatment and/or prevention of hair-loss.

As described above, the present inventors surprisingly found that the P2Y5 receptor plays a crucial role in maintenance of hair growth and that the dysfunction of this G protein-coupled receptor is one reason for hair-loss in humans. Thus, the polypeptide encoded by the above-described nucleic acid molecule may be used for the treatment and/or prevention of hair-loss. For example, administering the polypeptide of the invention as therapy to compensate for reduced or aberrant expression or activity of the protein may be useful in the treatment and/or prevention of hair-loss. The finding of the present invention that the P2Y5 receptor plays a role in hair growth/hair-loss has been further confirmed by genetical and molecular biological data published by Shimomura Y. et al. 2008, (Nat. Genet.; 40(3):335-9) as well as by Azeem Z, et al., 2008 (Hum Genet.; 123(5):515-9).

The invention further relates to a pharmaceutical composition comprising (i) a nucleic acid molecule encoding a polypeptide having P2Y5 receptor function wherein said nucleic acid molecule comprises (a) a nucleic acid molecule encoding a polypeptide having the amino acid sequence of SEQ ID NO:2; (b) a nucleic acid molecule having the DNA sequence of SEQ ID NO:1; (c) a nucleic acid molecule having the sequence of SEQ ID NO:1, wherein each thymidine is replaced by uridine; (d) a nucleic acid molecule that hybridizes under stringent conditions to the complementary strand of a nucleic acid molecule of (a), (b) or (c); (e) a nucleic acid molecule encoding a polypeptide that is at least 80% homologous to the polypeptide of (a); or (f) a nucleic acid molecule that is degenerate with respect to the nucleic acid molecule of (b), (c) or (d); (ii) a vector comprising the nucleic acid molecule of (i); (iii) a host cell comprising the vector of (ii); or (iv) a polypeptide encoded by the nucleic acid molecule of (i).

In accordance with the present invention, the term “pharmaceutical composition” relates to a composition for administration to a patient, preferably a human patient. The pharmaceutical composition of the invention comprises at least one of the compounds recited above. It may, optionally, comprise further molecules capable of altering the characteristics of the compounds of the invention thereby, for example, suppressing, stabilizing, blocking, modulating and/or activating their function. The composition may be in solid, liquid or gaseous form and may be, inter alia, in the form of (a) powder(s), (a) tablet(s), (a) solution(s) or (an) aerosol(s). The pharmaceutical composition of the present invention may, optionally and additionally, comprise a pharmaceutically acceptable carrier. Examples of suitable pharmaceutical carriers, are well known in the art and include phosphate buffered saline solutions, water, emulsions, such as oil/water emulsions, various types of wetting agents, sterile solutions, organic solvents including DMSO etc. Compositions comprising such carriers can be formulated by well known conventional methods. These pharmaceutical compositions can be administered to the subject at a suitable dose. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgement of the ordinary clinician or physician. Generally, the regimen as a regular administration of the pharmaceutical composition should be in the range of 1 μg to 5 g per day. However, a more preferred dosage might be in the range of 0.01 mg to 100 mg, even more preferably 0.01 mg to 50 mg and most preferably 0.01 mg to 10 mg per day.

The components of the pharmaceutical composition to be used for therapeutic administration must be sterile. Sterility is readily accomplished, e.g. by filtration through sterile filtration membranes (e.g., 0.2 micron membranes).

The components of the pharmaceutical composition ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous solution, and the resulting mixture is lyophilized. The infusion solution is prepared by reconstituting the lyophilized compound(s) using bacteriostatic Water-for-injection.

Preservatives and other additives may also be present such as, for example, antimicrobials, antioxidants, chelating agents, and inert gases and the like. Furthermore, the pharmaceutical composition may comprise further agents depending on the intended use of the pharmaceutical composition.

The components of the pharmaceutical composition as outlined in (i) to (iv) are as defined above.

The pharmaceutical composition may be particularly useful for the treatment or prevention of diseases associated with a dysfunction of the P2Y5 receptor, preferably diseases selected from those described herein above. As described hereinabove, P2Y5 receptor protein, or its corresponding nucleic acid molecule, plays a crucial role in the maintenance of hair growth while dysfunction of this G protein-coupled receptor gene is one reason for hair-loss in humans. Therefore, the pharmaceutical compositions of the invention offer the possibility to specifically target hair-loss in individuals suffering from these conditions. For example, overexpression of the nucleic acid molecules of the invention may be useful for “gene targeting” and/or “gene replacement” for restoring the mutant gene in affected individuals while the provision of the polypeptide of the invention may be used to compensate for reduced or aberrant expression or activity of the protein.

The invention further relates to a method of treating and/or preventing hair-loss comprising administering the pharmaceutical composition described above to a subject in need thereof.

In another embodiment, the present invention relates to a (i) nucleic acid molecule encoding a polypeptide having P2Y5 receptor function wherein said nucleic acid molecule comprises (a) a nucleic acid molecule encoding a polypeptide having the amino acid sequence of SEQ ID NO:2; (b) a nucleic acid molecule having the DNA sequence of SEQ ID NO:1; (c) a nucleic acid molecule having the sequence of SEQ ID NO:1, wherein each thymidine is replaced by uridine; (d) a nucleic acid molecule that hybridizes under stringent conditions to the complementary strand of a nucleic acid molecule of (a), (b) or (c); (e) a nucleic acid molecule encoding a polypeptide that is at least 80% homologous to the polypeptide of (a); or (f) a nucleic acid molecule that is degenerate with respect to the nucleic acid molecule of (b), (c) or (d); or (ii) a vector comprising the nucleic acid molecule of (i); or (iii) a host cell comprising the vector of (ii); or (iv) a polypeptide encoded by the nucleic acid molecule of (i) for use in treating and/or preventing hair-loss.

The present invention also relates to the use of (i) a nucleic acid molecule encoding a polypeptide having P2Y5 receptor function wherein said nucleic acid molecule comprises (a) a nucleic acid molecule encoding a polypeptide having the amino acid sequence of SEQ ID NO:2; (b) a nucleic acid molecule having the DNA sequence of SEQ ID NO:1; (c) a nucleic acid molecule having the sequence of SEQ ID NO:1, wherein each thymidine is replaced by uridine; (d) a nucleic acid molecule that hybridizes under stringent conditions to the complementary strand of a nucleic acid molecule of (a), (b) or (c); (e) a nucleic acid molecule encoding a polypeptide that is at least 80% homologous to the polypeptide of (a); or (f) a nucleic acid molecule that is degenerate with respect to the nucleic acid molecule of (b), (c) or (d); (ii) a vector comprising the nucleic acid molecule of (i); (iii) a host cell comprising the vector of (ii); or (iv) a polypeptide encoded by the nucleic acid molecule of (1) for the manufacture of a pharmaceutical composition for treating and/or preventing hair-loss.

The components for use for the manufacture of a pharmaceutical composition for treating and/or preventing hair-loss are as defined above.

The present invention also relates to a method for the identification of a compound useful in the treatment of hair-loss or as a lead compound for the development of an agent for treating hair-loss comprising the steps: (i) determining the level of P2Y5 receptor protein or P2RY5 transcript in a cell wherein said cell comprises P2RY5 DNA in expressible form; (ii) contacting said cell with a test compound; (iii) determining the level of P2Y5 receptor protein or P2RY5 transcript in said cell after contacting with the test compound; and (iv) comparing the P2Y5 receptor protein or P2RY5 transcript level determined in step (iii) with the P2Y5 receptor protein or P2RY5 transcript level determined in step (i), wherein an increase of P2Y5 receptor protein or P2RY5 transcript level in step (iii) as compared to step (i) indicates that the test compound is a compound useful in the treatment of hair-loss or as a lead compound for the development of an agent for treating hair-loss.

A “compound” in accordance with the present invention is, for example, a small molecule. Such a small molecule may be, for example, an organic molecule. Organic molecules relate to or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. Alternatively the compound may be an inorganic compound. Inorganic compounds are derived from mineral sources and include all compounds without carbon atoms (except carbon dioxide, carbon monoxide and carbonates).

Test compounds include but are not limited to, for example, purine analogs, peptides such as soluble peptides, including Ig-tailed fusion peptides and members of random peptide libraries (see, e.g., Lam et al. (1991) Nature 354: 82-84; Houghten et al. (1991) Nature 354: 84-86) and combinatorial chemistry-derived molecular libraries made of D- and/or L-configuration amino acids; phosphopeptides (e.g., members of random and partially degenerate, directed phosphopeptide libraries, see, e.g., Songyang et al. (1993) Cell 72: 767-778); antibodies (e.g., polyclonal, monoclonal, humanized, anti-idiotypic, chimeric, and single chain antibodies as well as Fab, F(ab′)2, Fab expression library fragments, and epitope-binding fragments of antibodies).

One example for a test compound is a soluble full-length receptor or fragment that competes for ligand binding. Other test compounds include mutant receptors or appropriate fragments containing mutations that affect receptor function and thus compete for ligand. Accordingly, a fragment that competes for a ligand, for example with a higher affinity, or a fragment that binds a ligand but does not allow release, is encompassed by the invention.

The term “P2RY5 DNA in expressible form” refers to P2RY5 DNA under the control of its naturally occurring regulatory sequences. For example, the P2RY5 DNA may be under the control of its natural promoter. Furthermore, P2RY5 DNA under the control of the fragments constituting the minimal promoter of P2RY5 is also envisaged by the invention.

As described hereinabove, the P2Y5 receptor is involved in processes that regulate the maintenance of hair growth, while dysfunction of this G protein-coupled receptor is one reason for hair-loss in humans. Therefore, the use of P2Y5 receptor as a target for the discovery of compounds that modulate processes of hair-loss is also encompassed by the present invention. It is envisaged that an increase of expression levels of P2RY5 conferred by a compound as described above may contribute to the maintenance of hair growth and may ameliorate conditions associated with dysfunction of this receptor, such as hair-loss. Accordingly, measurement of the P2Y5 receptor protein or P2RY5 transcript level may be used to determine the readout of the above-described assay.

The level of P2Y5 receptor protein or P2RY5 transcript may e.g. be undetectable before contacting the above-mentioned cell with the test compound and it may be clearly detectable after contacting the cell with the test compound indicating a compound suitable for the treatment of hair-loss or as a lead compound for the development of a compound for the treatment of hair-loss. Alternatively, the above-mentioned cell may exhibit a detectable level of P2Y5 receptor protein or P2RY5 transcript before contacting with the test compound and the level of P2Y5 receptor protein or P2RY5 transcript may be higher after contacting the cell with the test compound. Preferably, the level of P2Y5 receptor protein or P2RY5 transcript is for example at least 5, 10, 20, 30, 40 or 50% higher after contacting the cell with the test compound. More preferably, the level of P2Y5 receptor protein or P2RY5 transcript is for example at least 100, 200, 300 or 400% higher after contacting the cell with the test compound. Most preferably, the level of P2Y5 receptor protein or P2RY5 transcript is for example at least 500% higher after contacting the cell with the test compound.

Measurements of protein levels can be accomplished in several ways. Western blotting using specific antibodies or polyacrylamide gel electrophoresis (PAGE) in conjunction with protein staining techniques such as, but not limited to, Coomassie Brilliant blue or silver-staining may be used. Also of use in protein quantification is the Agilent Bioanalyzer technique.

Techniques for the determination of the transcript level include, but are not limited to RT-PCR and its various modifications such as qRT-PCR (also referred to as Real Time RT-PCR). PCR is well known in the art and is employed to make large numbers of copies of a target sequence. This is done on an automated cycler device, which can heat and cool containers with the reaction mixture in a very short time. The PCR, generally, consists of many repetitions of a cycle which consists of: (a) a denaturing step, which melts both strands of a DNA molecule and stalls all previous enzymatic reactions; (b) an annealing step, which is aimed at allowing the primers to anneal specifically to the melted strands of the DNA molecule; and (c) an extension step, which elongates the annealed primers by using the information provided by the template strand. Generally, PCR can be performed for example in a 50 μl reaction mixture containing 5 μl of 10×PCR buffer with 1.5 mM MgCl₂, 200 μM of each deoxynucleoside triphosphate, 0.5 μl of each primer (10 μM), about 10 to 100 ng of template DNA and 1 to 2.5 units of Taq Polymerase. The primers for the amplification may be labeled or be unlabeled. DNA amplification can be performed, e.g., with a model 2400 thermal cycler (Applied Biosystems, Foster City, Calif.): 2 min at 94° C., followed by 30 to 40 cycles consisting of annealing (e.g. 30 s at 50° C.), extension (e.g. 1 min at 72° C., depending on the length of DNA template and the enzyme used), denaturing (e.g. 10 s at 94° C.) and a final annealing step at 55° C. for 1 min as well as a final extension step at 72° C. for 5 min. Suitable polymerases for use with a DNA template include, for example, E. coli DNA polymerase I or its Klenow fragment, T4 DNA polymerase, Tth polymerase, Taq polymerase, a heat-stable DNA polymerase isolated from Thermus aquaticus, Amplitaq, Pfu and KOD, some of which may exhibit proof-reading function and/or different temperature optima. The person skilled in the art knows how to optimize PCR conditions for the amplification of specific nucleic acid molecules with primers of different length and/or composition or to scale down or increase the volume of the reaction mix.

The “reverse transcriptase polymerase chain reaction” (RT-PCR) is used when the nucleic acid to be amplified consists of RNA. The term “reverse transcriptase” refers to an enzyme that catalyzes the polymerization of deoxyribonucleoside triphosphates to form primer extension products that are complementary to a ribonucleic acid template. The enzyme initiates synthesis at the 3′-end of the primer and proceeds toward the 5′-end of the template until synthesis terminates. Examples of suitable polymerizing agents that convert the RNA target sequence into a complementary, copy-DNA (cDNA) sequence are avian myeloblastosis virus (AMV) reverse transcriptase and Thermus thermophilus DNA polymerase, a thermostable DNA polymerase with reverse transcriptase activity marketed by Perkin Elmer. Typically, the genomic RNA/cDNA duplex template is heat denatured during the first denaturation step after the initial reverse transcription step leaving the DNA strand available as an amplification template. High-temperature RT provides greater primer specificity and improved efficiency. U.S. patent application Ser. No. 07/746,121, filed Aug. 15, 1991, describes a “homogeneous RT-PCR” in which the same primers and polymerase suffice for both the reverse transcription and the PCR amplification steps, and the reaction conditions are optimized so that both reactions occur without a change of reagents. Thermus thermophilus DNA polymerase, a thermostable DNA polymerase that can function as a reverse transcriptase, can be used for all primer extension steps, regardless of template. Both processes can be done without having to open the tube to change or add reagents; only the temperature profile is adjusted between the first cycle (RNA template) and the rest of the amplification cycles (DNA template). The RT Reaction can be performed, for example, in a 20 μl reaction mix containing: 4 μl of 5×AMV-RT buffer, 2 μl of Oligo dT (100 μg/ml), 2 μl of 10 mM dNTPs, 1 μl total RNA, 10 Units of AMV reverse transcriptase, and H₂O to 20 μl final volume. The reaction may be, for example, performed by using the following conditions: The reaction is held at 70 C.° for 15 minutes to allow for reverse transcription. The reaction temperature is then raised to 95 C.° for 1 minute to denature the RNA-cDNA duplex. Next, the reaction temperature undergoes two cycles of 95° C. for 15 seconds and 60 C.° for 20 seconds followed by 38 cycles of 90 C.° for 15 seconds and 60 C.° for 20 seconds. Finally, the reaction temperature is held at 60 C.° for 4 minutes for the final extension step, cooled to 15 C.°, and held at that temperature until further processing of the amplified sample. Any of the above mentioned reaction conditions may be scaled up according to the needs of the particular case.

Real-time PCR employs a specific probe, in the art also referred to as TaqMan probe, which has a reporter dye covalently attached at the 5′ end and a quencher at the 3′ end. After the TaqMan probe has been hybridized in the annealing step of the PCR reaction to the complementary site of the polynucleotide being amplified, the 5′ fluorophore is cleaved by the 5′ nuclease activity of Taq polymerase in the extension phase of the PCR reaction. This enhances the fluorescence of the 5′ donor which was formerly quenched due to the close proximity to the 3′ acceptor in the TaqMan probe sequence. Thereby, the process of amplification can be monitored directly and in real time, which permits a significantly more precise determination of expression levels than conventional end-point PCR. Also of use in Real time RT-PCR experiments is a DNA intercalating dye such as SybrGreen for monitoring the de novo synthesis of double stranded DNA molecules.

In a preferred embodiment, the method is carried out in vitro.

In vitro methods offer the possibility of establishing high-throughput assays which are capable of screening up to several thousand compounds in parallel. High-throughput assays, independently of being biochemical, cellular or other assays, generally may be performed in wells of microtiter plates, wherein each plate may contain 96, 384 or 1536 wells. Handling of the plates, including incubation at temperatures other than ambient temperature, and bringing into contact of test compounds with the assay mixture is preferably effected by one or more computer-controlled robotic systems including pipetting devices. In case large libraries of test compounds are to be screened and/or screening is to be effected within short time, mixtures of, for example 10, 20, 30, 40, 50 or 100 test compounds may be added to each well. In case a well exhibits biological activity, said mixture of test compounds may be de-convoluted to identify the one or more test compounds in said mixture giving rise to said activity.

The identified so-called lead compounds may be optimized to arrive at a compound which may be used in a pharmaceutical composition. Methods for the optimization of the pharmacological properties of compounds identified in screens, the lead compounds, are known in the art and comprise methods of modifying a compound identified as a lead compound to achieve: (i) modified site of action, spectrum of activity, organ specificity, and/or (ii) improved potency, and/or (iii) decreased toxicity (improved therapeutic index), and/or (iv) decreased side effects, and/or (v) modified onset of therapeutic action, duration of effect, and/or (vi) modified pharmacokinetic parameters (resorption, distribution, metabolism and excretion), and/or (vii) modified physico-chemical parameters (solubility, hygroscopicity, color, taste, odor, stability, state), and/or (viii) improved general specificity, organ/tissue specificity, and/or (ix) optimized application form and route by (i) esterification of carboxyl groups, or (ii) esterification of hydroxyl groups with carboxylic acids, or (iii) esterification of hydroxyl groups to, e.g. phosphates, pyrophosphates or sulfates or hemi-succinates, or (iv) formation of pharmaceutically acceptable salts, or (y) formation of pharmaceutically acceptable complexes, or (vi) synthesis of pharmacologically active polymers, or (vii) introduction of hydrophilic moieties, or (viii) introduction/exchange of substituents on aromates or side chains, change of substituent pattern, or (ix) modification by introduction of isosteric or bioisosteric moieties, or (x) synthesis of homologous compounds, or (xi) introduction of branched side chains, or (xii) conversion of alkyl substituents to cyclic analogues, or (xiii) derivatisation of hydroxyl group to ketales, acetates, or (xiv) N-acetylation to amides, phenylcarbamates, or (xv) synthesis of Mannich bases, imines, or (xvi) transformation of ketones or aldehydes to Schiff's bases, oximes, acetates, ketales, enolesters, oxazolidines, thiazolidines or combinations thereof.

The various steps recited above are generally known in the art. They include or rely on quantitative structure-activity relationship (QSAR) analyses (Kubinyi, “Hausch-Analysis and Related Approaches”, VCH Verlag, Weinheim, 1992), combinatorial biochemistry, classical chemistry and others (see, for example, Holzgrabe and Bechtold, Deutsche Apotheker Zeitung 2000, 140(8): 813).

Any of the cells described herein above may be used for the method of the invention. Preferably, said cell is a HaCaT cell (immortalized keratinocyte cell line), primary keratinocyte or primary hair follicle cell.

In another preferred embodiment of the method of the invention said cell comprises a nucleic acid molecule encoding a protein having P2Y5 receptor function fused to a reporter gene, wherein said nucleic acid molecule comprises (a) a nucleic acid molecule encoding a polypeptide having the amino acid sequence of SEQ ID NO:2; (b) a nucleic acid molecule having the DNA sequence of SEQ ID NO:1; (c) a nucleic acid molecule having the sequence of SEQ ID NO:1, wherein each thymidine is replaced by uridine; (d) a nucleic acid molecule that hybridizes under stringent conditions to the complementary strand of a nucleic acid molecule of (a), (b) or (c); (e) a nucleic acid molecule encoding a polypeptide having at least 80% sequence identity to the polypeptide of (a); or (f) a nucleic acid molecule that is degenerate with respect to the nucleic acid molecule of (b), (c) or (d).

The fusion to a reporter gene allows the indirect measurement of protein level by measuring the level of the reporter gene attached to the protein of interest. Examples of reporter genes include, but are not limited to, green fluorescent protein (GFP), yellow fluorescent protein (YFP), red fluorescent protein (RFP), cyan fluorescent protein (CFP), luciferase or β-galactosidase.

In an alternative embodiment, the present invention relates to a method for the identification of a compound useful in the treatment of hair-loss or as a lead compound for the development of an agent for treating hair-loss comprising the steps: (i) contacting a cell containing P2Y5 receptor protein and a P2Y5 target molecule with a test compound; and (ii) determining the level of activity of the P2Y5 target molecule before contacting the P2Y5 protein with the test compound and after contacting the P2Y5 protein with the test compound, wherein a modulation of activity of the target molecule after contacting the P2Y5 protein with the test compound as compared to the level before contacting the P2Y5 protein with the test compound indicates that the test compound is a compound useful in the treatment of hair-loss or as a lead compound for the development of an agent for treating hair-loss.

In a preferred embodiment of the method of the invention, the compound modulates P2Y5 receptor activity.

Such compounds may, for example, alter the affinity or rate of binding to a known ligand, compete with a ligand for binding to the receptor, or displace a ligand bound to the receptor.

The term “P2Y5 target molecule” refers to molecules that are affected by P2Y5 receptor activity as a result of the downstream signalling of this G-protein coupled receptor. Downstream signalling refers to the modulation (e.g., stimulation or inhibition) of a cellular function/activity upon binding of a ligand to the GPCR. Examples of such functions include mobilization of intracellular molecules that participate in a signal transduction pathway, for example cAMP or adenylate cyclase or that result in a change of intracellular molecules, for example release of calcium, polarisation of the plasma membrane, production or secretion of molecules, alteration in the structure of a cellular component, cell proliferation, cell differentiation and cell survival. The response mediated by the receptor protein is cell type dependent and may, for example, stimulate an activity such as release of compounds, gating of a channel, cellular adhesion, migration, differentiation, etc., through cyclic AMP metabolism and turnover while in other cells, the binding of the ligand will produce a different result.

The P2Y5 target molecule can be a ligand or a component of the signal pathway with which the receptor protein normally interacts, for example, a G-protein or other interactor involved in cAMP or phosphatidylinositol turnover and/or adenylate cyclase, or phospholipase C activation.

The assay includes the steps of combining the receptor protein with a test compound under conditions that allow the receptor protein to interact with the P2Y5 target molecule, and to detect the formation of a complex between the protein and the target or to detect the biochemical consequence of the interaction with the receptor protein and the target, such as any of the associated effects of signal transduction such as G-protein phosphorylation, cyclic AMP or phosphatidylinositol turnover, and adenylate cyclase or phospholipase C activation.

Furthermore, assays determining the expression of genes that are up- or down-regulated in response to the receptor protein dependent signal cascade can be employed. For example, the regulatory region of such genes may be operably linked to a marker that is easily detectable, such as for example luciferase, green fluorescent protein (GFP), yellow fluorescent protein (YFP), red fluorescent protein (RFP), cyan fluorescent protein (CFP) or β-galactosidase. Alternatively, phosphorylation of the receptor protein, or a receptor protein target, may also be measured.

In a preferred embodiment of the method of the invention, the P2Y5 target molecule is cAMP.

Intracellular levels of cAMP can be determined using methods well known in the art, for example using Flash-plate kits (Dupont) or the Biotrak cAMP SPA screening assay system (Amersham) according to the manufacturer's instructions (George et al., Journal of Biomolecular Screening 2, 4:235 (1997)).

Binding and/or activating compounds can also be screened by using chimeric receptor proteins in which the amino terminal extracellular domain, or parts thereof, the entire transmembrane domain or subregions, such as any of the seven transmembrane segments or any of the intracellular or extracellular loops and the carboxy terminal intracellular domain, or parts thereof, can be replaced by heterologous domains or subregions. For example, a G protein-binding region can be used that interacts with a different G protein than that which is recognized by the native receptor. Accordingly, a different set of signal transduction components is available as an end-point assay for activation. Alternatively, the entire transmembrane portion or subregions (such as transmembrane segments or intracellular or extracellular loops) can be replaced with the entire transmembrane portion or subregions specific to a host cell that is different from the host cell from which the amino terminal extracellular domain and/or the G protein-binding region are derived. This allows for assays to be performed in other than the specific host cell from which the receptor is derived. Alternatively, the amino terminal extracellular domain (and/or other ligand-binding regions) could be replaced by a domain (and/or other binding region) binding a different ligand, thus, providing an assay for test compounds that interact with the heterologous amino terminal extracellular domain (or region) but still cause signal transduction. Finally, activation can be detected by a reporter gene containing an easily detectable coding region operably linked to a transcriptional regulatory sequence that is part of the native signal transduction pathway.

The receptor polypeptides are further useful in competition binding assays in methods designed to discover compounds that interact with the receptor. Thus, a compound is exposed to a receptor polypeptide under conditions that allow the compound to bind or to otherwise interact with the polypeptide. Soluble receptor polypeptide is also added to the mixture. If the test compound interacts with the soluble receptor polypeptide, it decreases the amount of complex formed or activity from the receptor target. This type of assay is particularly useful in cases in which compounds are sought that interact with specific regions of the receptor. Thus, the soluble polypeptide that competes with, the target receptor region is designed to contain peptide sequences corresponding to the region of interest.

The P2Y5 receptor is, as described above, involved in processes that regulate the maintenance of hair growth and dysfunction of this G protein-coupled receptor is one reason for hair-loss in humans. Therefore, its use as a target for the discovery of compounds that increase the activity of P2Y5 is also envisaged. P2Y5 protein is useful, as has been surprisingly found in accordance with the present invention, for the prevention of hair-loss and therefore, increase of its activity by the use of compounds identified in the above-described screen will result in amelioration or even loss of symptoms associated with hair-loss.

In a further embodiment, the present invention relates to a nucleic acid molecule deviating from the nucleic acid molecules described above by at least one mutation, wherein said mutation results in a loss of function of the polypeptide encoded by the nucleic acid molecule described above and is selected from: (I) a substitution; (ii) a deletion; (iii) an inversion; and/or (iv) an insertion; and wherein said mutation is causative and/or indicative of hair-loss.

The term “mutation” in accordance with the invention refers to changes to the nucleotide sequence of the genetic material and includes deletion, insertion, or substitution of one or more nucleotides in the gene, chromosomal rearrangement, such as inversion or transposition, modification of genomic DNA, such as aberrant methylation patterns or changes in gene copy number, such as amplification.

The “nucleic acid molecule deviating from the nucleic acid molecules described above by at least one mutation” in accordance is also referred to herein as the “mutant nucleic acid molecule”.

The term “substitution” in accordance with the present invention, refers to point mutations preferably resulting in an amino acid exchange.

The term “deletion” as used in accordance with the present invention refers to the loss of nucleotides.

The term “inversion” in accordance with the present invention refers to a kind of mutation in which the order of the nucleotides in a section of the nucleic acid molecule is reversed with respect to the remainder of the nucleic acid molecule.

The term “insertion” in accordance with the present invention refers to the addition of one or more nucleotides to a nucleic acid molecule, wherein the addition is not to the 5′ or 3′ end of the nucleic acid molecule.

All of the above mutations may lead to the creation of nonsense codons, i.e. stop codons, which will lead to premature termination of translation and, thus, to truncated forms of the polypeptide of the present invention. Furthermore, such mutations, in particular deletions or insertions, may lead to a frameshift mutation, causing all of the codons occurring after the mutation to be read incorrectly during translation. This frameshift may result in the production of a severely altered and potentially nonfunctional protein. The skilled person knows how to assess whether a mutation in the nucleic acid molecule encoding the P2Y5 receptor leads to a loss of function of this polypeptide. Non-limiting examples include immunofluorescence assays, that may be performed to determine the localisation of the polypeptide, whereas an incorrect localisation, such as for example to the endoplasmic reticulum, is indicative of a loss of function of the membrane receptor polypeptide P2Y5. Furthermore, employing bioinformatic means the skilled person can predict whether the mutation is located within domain 2 or 3 of the polypeptide. Said domains are necessary for the activation of the G-protein downstream of the GPCR (Johnston and Siderovski, 2007, Mol. Pharmacol. 2007 August; 72(2):219-30. Also CRE luciferase reporter assays may be employed, as confirmed by Pasternack S M et al., 2008, Nat. Genetics 40(3):329-34.

Especially with respect to substitutions and deletions, it could be shown in accordance with the present invention that such mutations may lead to a frame shift which in turn leads to the expression of a truncated form of the polypeptide of the present invention due to the generation of a premature stop codon.

Thus, detection of a mutated form of the P2RY5 gene associated with a dysfunction of the P2Y5 receptor protein provides a diagnostic tool for diseases or susceptibilities to diseases related to P2Y5 receptor activity, in particular the diseases cited herein above. Furthermore, the nucleic acid molecule of the invention may also be used for testing an individual for a genotype that, while not necessarily causing the disease, nevertheless affects the treatment modality. Thus, the polynucleotides can be used to study the relationship between an individual's genotype and the individual's response to a compound used for treatment (pharmacogenomic relationship). In the present case, for example, a mutation in the receptor gene that results in altered affinity for ligand could result in an excessive or decreased drug effect with standard concentrations of ligand that activates the receptor. Accordingly, the nucleic acid molecules of the invention can be used to assess the mutation content of the receptor gene in an individual in order to select an appropriate compound or dosage regimen for treatment.

In a preferred embodiment of the present invention, said substitution is a cytosine to thymidine exchange at a nucleotide position corresponding to position 463 of the nucleotide sequence of SEQ ID NO:1. The nucleic acid molecule having this cytosine to thymidine exchange is shown in SEQ ID NO:5.

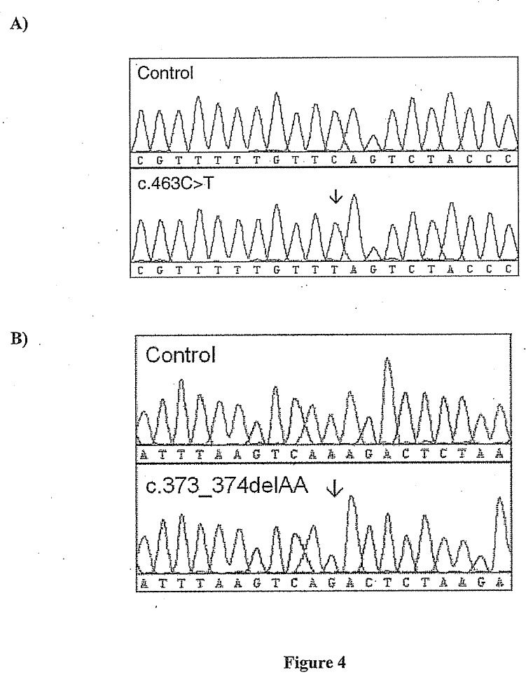

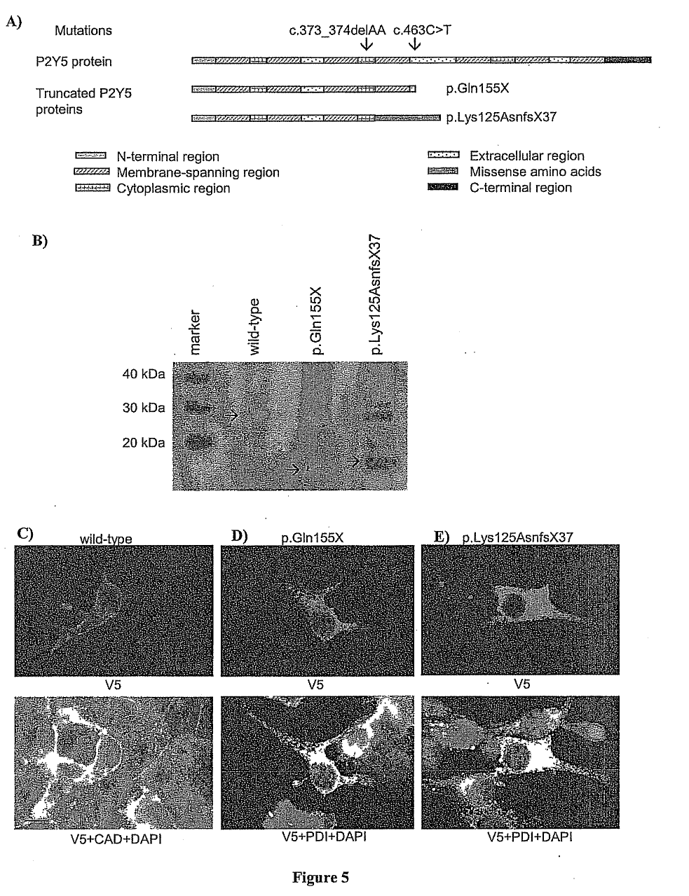

As shown in the examples, direct sequencing of P2RY5 in individuals from a Saudi-Arabian family suffering from autosomal-recessive hypotrichosis simplex (family 1) revealed a substitution of a cytosine to thymidine at the nucleotide position 463 of P2RY5 (c.463C>T; according to position 463 of SEQ ID NO:1), which results in a nonsense mutation leading to premature termination of translation (p.Gln155X; SEQ ID NO:6) (FIGS. 4A and 5A). This mutation was not detected in 506 control chromosomes which included 138 chromosomes of Arabian origin.

In another preferred embodiment of the present invention, said deletion is a deletion of the adenosine at a nucleotide position corresponding to position 373 of the nucleotide sequence of SEQ ID NO:1 and/or a deletion of the adenosine at a nucleotide position corresponding to position 374 of the nucleotide sequence of SEQ ID NO:1. The nucleic acid molecule having this deletion at position 373 and 374 is shown in SEQ ID NO:7.

Sequencing of P2RY5 in two additional HS-families from Saudi-Arabia (families 2 and 3) revealed the deletion of two adenosines from position 373 and 374 of P2RY5 (c.373_{—}374delAA, corresponding to the positions in SEQ ID NO:1) leading to a frame-shift and premature termination of translation of the P2Y5 receptor protein (p.Lys125AsnfsX37; SEQ ID NO:8) (FIGS. 4B and 5A). Due to the frame shift the resulting mutated polypeptide comprises 36 altered amino acids before the premature stop codon. Also this mutation was not detected in 506 control chromosomes which included 138 chromosomes of Arabian origin.

In a further embodiment, the invention also relates to a vector comprising the mutant nucleic acid molecule of the invention as described above.

The term “vector” in accordance with the present invention has been described above.

In a further embodiment, the present invention also relates to a host genetically engineered with the mutant nucleic acid molecule of the invention or with the vector comprising the mutant nucleic acid molecule of the invention as described above, Said host may be produced by introducing said vector into a host which upon its presence mediates the expression of the polypeptide.

The host may be any prokaryote or eukaryotic cell. Suitable prokaryotes/bacteria are those generally used for cloning like E. coli (e.g., E. coli strains BL21(DE3), HB101, DH5α, XL1 Blue, Y1090 and JM101), Salmonella typhimurium, Serratia marcescens, Pseudomonas putida, Pseudomonas fluorescens, Streptomyces lividans, Lactococcus lactis, Mycobacterium smegmatis or Bacillus subtilis. As described above, suitable eukaryotic hosts may be animal cells such as mammalian cells, amphibian cells, fish cells, insect cells such as Drosophila S2 and Spodoptera Sf9 cells, fungal cells, plant cells, transgenic non-human animals or transgenic plants.

A method for the production of a transgenic non-human animal, for example transgenic mouse, comprises introduction of the aforementioned nucleic acid molecule or targeting vector of the invention into a germ cell, an embryonic cell, stem cell or an egg or a cell derived thereof. The non-human animal can be used in accordance with the invention in a method for identification of compounds, described herein above in connection with cells. Production of transgenic embryos and screening of those can be performed, e.g., as described by A. L. Joyner Ed., Gene Targeting, A Practical Approach (1993), Oxford University Press. The DNA of the embryonic membranes of embryos can be analyzed using, e.g., Southern blots with an appropriate probe; see supra. A general method for making transgenic non-human animals is described in the art, see for example WO 94/24274. For making transgenic non-human organisms (which include homologously targeted non-human animals), embryonal stem cells (ES cells) are preferred. Murine ES cells, such as AB-1 line grown on mitotically inactive SNL76/7 cell feeder layers (McMahon and Bradley, Cell 62:1073-1085 (1990)) essentially as described (Robertson, E. J. (1987) in Teratocarcinomas and Embryonic Stem Cells: A Practical Approach. E. J. Robertson, ed. (Oxford: IRL Press), p. 71-112) may be used for homologous gene targeting. Other suitable ES lines include, but are not limited to, the E14 line (Hooper et al., Nature 326:292-295 (1987)), the D3 line (Doetschman et al., J. Embryol. Exp. Morph. 87:27-45 (1985)), the CCE line (Robertson et al., Nature 323:445-448 (1986)), and the AK-7 line (Zhuang et al., Cell 77:875-884 (1994)). The success of generating a mouse line from ES cells bearing a specific targeted mutation depends on the pluripotence of the ES cells (i.e., their ability, once injected into a host developing embryo, such as a blastocyst or morula, to participate in embryogenesis and contribute to the germ cells of the resulting animal). The blastocysts containing the injected ES cells are allowed to develop in the uteri of pseudopregnant non-human females and are born, e.g. as chimeric mice. The resultant transgenic mice are chimeric for cells having either the recombinase or reporter loci and are backcrossed and screened for the presence of the correctly targeted transgene(s), usually by PCR or Southern blot analysis on tail biopsy DNA of offspring so as to identify transgenic mice heterozygous for either the recombinase or reporter locus/loci.

The transgenic non-human animals may, for example, be transgenic mice, rats, hamsters, dogs, monkeys, rabbits, pigs, or cows. Preferably, said transgenic non-human animal is a mouse.

In an additional-embodiment, the present invention relates to a method of producing a polypeptide of the invention comprising culturing a host according to the invention under suitable conditions and isolating the polypeptide of the invention produced from said host or culture.

A large number of suitable methods exist in the art to produce polypeptides in appropriate hosts. If the host is a unicellular organism such as a prokaryote, a mammalian or insect cell, the person skilled in the art can revert to a variety of culture conditions. Conveniently, the produced protein is harvested from the culture medium, lysates of the cultured organisms or from isolated (biological) membranes by established techniques. In the case of a multicellular organism, the host may be a cell which is part of or derived from a part of the organism, for example said host cell may be the harvestable part of a plant. A preferred method involves the recombinant production of protein in hosts as indicated above. For example, nucleic acid sequences comprising the nucleic acid molecule according to the invention can be synthesized by PCR and inserted into an expression vector. Subsequently a suitable host may be transformed with the expression vector. Thereafter, the host is cultured to produce the desired polypeptide, which is isolated and purified. Such methods are well known in the art (see, e.g., Sambrook and Russel “Molecular Cloning, A Laboratory Manual”, Cold Spring Harbor Laboratory, N.Y. (2001)). An alternative method for producing the polypeptide of the invention is in vitro translation of mRNA: Suitable cell-free expression systems for use in accordance with the present invention include rabbit reticulocyte lysate, wheat germ extract, canine pancreatic microsomal membranes, E. coli S30 extract, and coupled transcription/translation systems such as the TNT-system (Promega). These systems allow the expression of recombinant polypeptides upon the addition of cloning vectors, DNA fragments, or RNA sequences containing coding regions and appropriate promoter elements.

In addition to recombinant production, fragments of the protein or the fusion protein of the invention may e.g. be produced by direct peptide synthesis using solid-phase techniques (cf Stewart et al. (1969) Solid Phase Peptide Synthesis; Freeman Co, San Francisco; Merrifield, J. Am. Chem. Soc. 85 (1963), 2149-2154).

Synthetic protein synthesis may be performed using manual techniques or by automation. Automated synthesis may be achieved, for example, using the Applied Biosystems 431A Peptide Synthesizer (Perkin Elmer, Foster City Calif.) in accordance with the instructions provided by the manufacturer. Various fragments may be chemically synthesized separately and combined using chemical methods to produce the full length molecule. As indicated above, chemical synthesis, such as the solid phase procedure described by Houghton (Proc. Natl. Acad. Sci., 1985, 82: 5131) can be used.

Protein isolation and purification can be achieved by any one of several known techniques; for example and without limitation, ion exchange chromatography, gel filtration chromatography, affinity chromatography, high pressure liquid chromatography (HPLC), reversed phase HPLC, hydrophobic interaction chromatography and preparative disc gel electrophoresis. Protein isolation/purification techniques may require modification of the polypeptides of the present invention using conventional methods. For example, a histidine tag can be further added to the protein to allow purification on a nickel column. Other modifications may cause higher or lower activity, permit higher levels of protein production, or simplify purification of the protein.

In an alternative embodiment the invention provides a polypeptide encoded by the nucleic acid molecule of the invention or produced by the method of the invention. The polypeptide encoded by the mutant nucleic acid molecule of the invention is also referred to herein as the “mutant polypeptide”.

In a further embodiment the invention provides an antibody, aptamer or phage that specifically binds to the nucleic acid molecule or the polypeptide of the invention. Said antibody may be, inter alia, a monoclonal or a polyclonal antibody.

The term “antibody” includes monoclonal antibodies, polyclonal antibodies, single chain antibodies, or fragments thereof that specifically bind said peptide or polypeptide, also including bispecific antibodies, synthetic antibodies, antibody fragments, such as Fab, a F(ab₂)′, Fv or scFv fragments etc., or a chemically modified derivative of any of these. Monoclonal antibodies can be prepared, for example, by the techniques as originally described, in Köhler and Milstein, Nature 256 (1975), 495, and Gerfré, Meth. Enzymol. 73 (1981), 3, which comprise the fusion of mouse myeloma cells to spleen cells derived from immunized mammals with modifications developed by the art. Furthermore, antibodies or fragments thereof to the aforementioned peptides can be obtained by using methods which are described, e.g., in Harlow and Lane “Antibodies, A Laboratory Manual”, CSH Press, Cold Spring Harbor, 1988. When derivatives of said antibodies are obtained by the phage display technique, surface plasmon resonance as employed in the BIAcore system can be used to increase the efficiency of phage antibodies which bind to an epitope of the peptide or polypeptide of the invention (Schier, Human Antibodies Hybridomas 7 (1996), 97-105; Malmborg, J. Immunol. Methods 183 (1995), 7-13). The production of chimeric antibodies is described, for example, in WO89/09622. A further source of antibodies to be utilized in accordance with the present invention are so-called xenogenic antibodies. The general principle for the production of xenogenic antibodies such as human antibodies in mice is described in, e.g., WO 91/10741, WO 94/02602, WO 96/34096 and WO 96/33735. Antibodies to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook, Molecular Cloning: A Laboratory Manual, Cold Spring Harbor Laboratory, Cold Spring Harbor, N.Y., 1989. The term “monoclonal” or “polyclonal antibody” (see Harlow and Lane, (1988), loc. cit.) also relates to derivatives of said antibodies which retain or essentially retain their binding specificity. Preferred derivatives of such antibodies are chimeric antibodies comprising, for example, a mouse or rat variable region and a human constant region.

The term “scFv fragment” (single-chain Fv fragment) is well understood in the art and preferred due to its small size and the possibility to recombinantly produce such fragments.

Preferably, the antibody, aptamer, fragment or derivative thereof according to the invention specifically binds the target protein, polypeptide or fragment or epitope thereof whose presence or absence is to be monitored.

The term “specifically binds” used in accordance with the present invention means that the antibody etc. does not or essentially does not cross-react with polypeptides of similar structures, such as wild type P2Y5, the sequence of which is shown in SEQ ID NO:2. Cross-reactivity of a panel of antibodies etc. under investigation may be tested, for example, by assessing binding of said panel of antibodies etc. under conventional conditions (see, e.g., Harlow and Lane, (1988), loc. cit.) to the polypeptide of interest as well as to a number of more or less (structurally and/or functionally) closely related polypeptides. Only those antibodies that bind to the polypeptide of interest but do not or do not essentially bind to any of the other polypeptides which are preferably expressed by the same tissue as the polypeptide of interest, are considered specific for the polypeptide of interest and selected for further studies in accordance with the method of the invention.

Aptamers are DNA or RNA molecules that bind other molecules, such as nucleic acids, proteins, small organic compounds, and even entire organisms. A database of aptamers is maintained at http://aptamer.icmb.utexas.edu/.

More specifically, aptamers can be classified as DNA or RNA aptamers or peptide aptamers. Whereas the former consist of (usually short) strands of oligonucleotides, the latter consist of a short variable peptide domain, attached at both ends to a protein scaffold.

Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. Peptide aptamers are proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable loop length is typically comprised of 10 to 20 amino acids, and the scaffold may be any protein which have good, solubility properties. Currently, the bacterial protein Thioredoxin-A is the most used scaffold protein, the variable loop being inserted within the reducing active site, which is a -Cys-Gly-Pro-Cys-loop in the wild protein, the two cysteins lateral chains being able to form a disulfide bridge. Peptide aptamer selection can be made using different systems, but the most used is currently the yeast two-hybrid system.

Aptamers offer the utility for biotechnological and therapeutic applications as they offer molecular recognition properties that rival those of the commonly used biomolecules, in particular antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications.

Non-modified aptamers are cleared rapidly from the bloodstream, with a half-life of minutes to hours, mainly due to nuclease degradation and clearance from the body by the kidneys, a result of the aptamer's inherently low molecular weight. Unmodified aptamer applications currently focus on treating transient conditions such as blood clotting, or treating organs such as the eye where local delivery is possible, This rapid clearance can be an advantage in applications such as in vivo diagnostic imaging. Several modifications, such as 2′-fluorine-substituted pyrimidines, polyethylene glycol (PEG) linkage, etc. are available to scientists with which the half-life of aptamers easily can be increased to the day or even week time scale.

Phages in accordance with the present invention refer to recombinant phages and are well known in the art and are described, for example, in Griffiths, A. D. at al.: EMBO J. 1994, 13:3245. The phage may carry immunoglobulin fragments or derivatives with a desired binding specificity for the polypeptide of the invention as a fusion protein on their surface, wherein the fusion partner is a surface molecule of the phage.

In a preferred embodiment of the method of the invention said antibody or aptamer or phage is detectably labeled. Whereas the aptamers are preferably radioactively labeled with ³H or ³²P or with a fluorescent marker such as described above, the phage or antibody may e.g. be labeled in a corresponding manner (with ¹³¹I as the preferred radioactive label) or be labeled with a tag such as His-tag, FLAG-tag or myc-tag.

In another aspect, the present invention provides an oligo- or polynucleotide comprising or consisting of an oligo- or polynucleotide selected from the group consisting of: (a) an oligo- or polynucleotide consisting of at least 10 consecutive nucleotides of SEQ ID NO:5 or 7; wherein the at least 10 consecutive nucleotides contain a T at position 463 of SEQ ID NO:5, or a G at position 373 of SEQ ID NO:7; (b) an oligo- or polynucleotide hybridizing under stringent conditions to at least a portion of the oligo- or polynucleotide of (a), wherein said portion comprises the nucleotide in position 463 of SEQ ID NOs:5 or the nucleotide in position 373 of SEQ ID NO:7, wherein said oligo- or polynucleotide contains a T at position 463 of SEQ ID NO:5 or a G at position 373 of SEQ ID NO:7; and (c) an oligo- or polynucleotide identical to the oligo- or polynucleotide of (a) or (b) with the exception that T is replaced by U.

In the context of the present invention the term “oligo- or polynucleotide” refers to nucleic acid molecules of different length: an oligonucleotide is a nucleic acid molecule consisting of up to 30 bp, a polynucleotide is a nucleic acid molecule consisting of more than 30 bp. The oligo- or polynucleotides comprise most preferably at least 17 nucleotides, but may also comprise at least 19, 25, 50, 100, 150, 200 or more nucleotides, whereas oligonucleotides of 10, 11, 12, 13, 14, 15 or 16 nucleotides are also envisaged. The oligo- or polynucleotides of the invention contain either a T at the nucleotide position corresponding to position 463 of SEQ ID NO:5 or a G at the nucleotide position corresponding to position 373 of SEQ ID NO:7, wherein the said nucleotides may be located at any position within the oligo- or polynucleotides. The oligo- or polynucleotides of the present invention are characterized as being associated with P2Y5-mediated diseases, in particular the diseases recited herein above. The G at position 373 of SEQ ID NO:7 is a result of the deletion of two adenosine residues from the wild type nucleic acid molecule at position 373 and 374.

Said complementary oligo- or polynucleotides of item (b) refer to oligo- or polynucleotides the sequence of which is uniquely fitting to (hybridizing to/complementary to preferably 100%) the sequences of the oligo- or polynucleotides described in (a), but not to wild type sequences, which contain a C at the position corresponding to position 463 of SEQ ID NO:5 and an A at the position corresponding to position 373 of SEQ ID NO:5 (as shown in SEQ ID NO:1). Upon hybridization of the oligo- or polynucleotides under stringent conditions to at least a portion of the oligo- or polynucleotide of (a), the nucleotides reflecting the mutations in the P2RY5 gene, i.e. the T at position 463 of SEQ ID NO:5 and/or the G at position 373 of SEQ ID NO:7, have to be contained in the resulting double-strand nucleic acid strand. The sequences of SEQ ID NO:5 and 7 correspond to the coding DNA of the mutant nucleic acid molecules comprising the mutations as compared to the wild type sequence of SEQ ID NO:1. The skilled person knows that also the corresponding mRNA (as represented in SEQ ID NO:3) or the genomic DNA (as shown in SEQ ID NO:4) may be analysed for the absence or presence of mutations.

These oligo- or polynucleotides of the invention may, for example, be useful as probes in Northern or Southern Blot analysis of RNA or DNA preparations, respectively, or can be used as oligonucleotide primers in PCR analysis dependent on their respective size. Also comprised by the invention are hybridizing oligo- or polynucleotides which are useful for analysing DNA-Protein interactions via, e.g., electrophoretic mobility shift analysis (EMSA). Preferably, said hybridizing oligo- or polynucleotides comprise at least 10, more preferably at least 15 nucleotides in length while a hybridizing oligo- or polynucleotide of the present invention to be used as a probe preferably comprises at least 100, more preferably at least 200, or most preferably at least 500 nucleotides in length, wherein the mutations of the present invention preferably have a central location. “Central” meaning most preferably that an equal number of nucleotides is located in the 5′- and 3′-direction adjacent to the position of the mutation. In a different preferred embodiment 60, 70, 80 or 90 percent of nucleotides are located in the 5′- or 3′-direction adjacent to the position of the mutation and the remaining nucleotides are located in the opposite direction.

The person skilled in the art is familiar with the preparation and the use of said probes (see, e.g., Sambrook and Russel “Molecular Cloning, A Laboratory Manual”, Cold Spring Harbor Laboratory, N.Y. (2001)). Said nucleic acid molecules may be chemically synthesized or transcribed by an appropriate vector containing a chimeric gene which allows for the transcription of said nucleic acid molecule in the cell.

Such oligo- or polynucleotides may be used in any of the above described methods. In particular, they may be used in distinguishing nucleic acid molecules carrying the mutations resulting in P2Y5 receptor truncation from nucleic acid molecule encoding the wild-type P2Y5 receptor. Therefore, these oligo- or polynucleotides are of relevance in the diagnosis of diseases associated with P2Y5 receptor mutation, such as hair-loss.

The invention further relates to a diagnostic composition comprising the nucleic acid molecule or the polypeptide according to the invention or the antibody, aptamer or phage of the invention.

The term “diagnostic composition” relates to compositions for diagnosing individual patients for their potential response to or curability by the pharmaceutical compositions of the invention and for diagnosing a predisposition of individual patients for a disease involving P2Y5 receptor dysfunction, such as hair-loss. The diagnostic composition of the invention comprises at least one of the compounds recited above. Said composition may further comprise appropriate buffer(s), and enzymes such as reverse transcriptase, thermostable polymerases etc. The diagnostic composition of the invention may be used to test for the presence of the nucleic acid molecule of the invention using methods well known in the art. Such methods are described, e.g. in Sambrook and Russel “Molecular Cloning, A Laboratory Manual”, Cold Spring Harbor Laboratory, N.Y. (2001).

Methods for testing a sample for the presence of the nucleic acid molecule of the invention include, but are not limited to, nucleic acid amplification, sequencing or hybridization assays.

Examples for nucleic acid amplification assays and means to perform such include without limitation PCR, (including nested PCR, RT-PCR, PCR extension assays, Nucleic Acid Sequence Base Amplification (NASBA), single-strand confirmation polymorphism (SSCP) PCR), amplification refractory mutation systems (ARMSTM) and amplification refractory mutation system linear extension (ALEXTM) assays. Details of such methods can be found in art, see, for example, Newton et al., Nucleic Acids Res, 17 (1989) 2503-2516; Agrawal (Ed.), “Protocols for Oligonucleotides and Analogs: Synthesis and Properties (Methods in Molecular Biology, 20)”, Humana Press, 1993; Hague et al., Diagn. Mol. Pathol. 7 (1998) 248-252; Innis et al. (Ed.), “PCR Applications: Protocols for Functional Genomics”, Academic Press, 1999; Chen and Janes (Ed.), “PCR Cloning Protocols: From Molecular Cloning to Genetic”, 2nd edition, Humana Press, 2002; Pissard et al., Clin. Chem. 48 (2002) 769-772; Steemers et al., Nature Meth. 3 (2006) 31-33; Kakavas et al., J. Clin. Lab. Anal. 20 (2006) 1-7.

Examples for sequencing assays comprise without limitation approaches of sequence analysis by direct sequencing, fluorescent SSCP in an automated DNA sequencer and Pyrosequencing. These procedures are common in the art, see e.g. Adams et al. (Ed.), “Automated DNA Sequencing and Analysis”, Academic Press, 1994; Alphey, “DNA Sequencing: From Experimental Methods to Bioinformatics”, Springer Verlag Publishing, 1997; Ramon et al., J. Transl. Med. 1 (2003) 9; Meng et al., J. Clin. Endocrinol. Metab. 90 (2005) 3419-3422.

Examples for hybridization assays comprise without limitation Northern and Southern blot assays, heteroduplex analysis, detection of mutations by sequence specific oligonucleotide hybridization, allele-specific oligonucleotide hybridization on DNA chips, assays based on Illumina's® technology, assays based on the BeadArray® technology, see, for example, Barnes et al., Nucleic Acids Res. 33 (2005) 5914-5923; Fan et al., Biotechniques 39 (2005) 583-588; Shen et al., Mutat. Res.-Fund. Mol. M. 573 (2005) 70-82; Steemers and Gunderson, Pharmacogenomics, 6 (2005) 777-782.

Examples for assays based on protein detection include without limitation method steps such as ion exchange chromatography, gel filtration chromatography, affinity chromatography, hydrophobic interaction chromatography, reversed phase HPLC, disc gel electrophoresis, capillary electrophoresis, Western blot analysis, immunoprecipitation, immuno-blotting, ELISA, RIA, indirect immunofluorescence experiments, amino acid sequencing, spectroscopic methods (UV, CD; IR, Fluoreszenz) and mass spectrometry (e.g. MS-QTOF), see, for example, Soejima and Koda, Transfusion 45 (2005) 1934-1939; Yeh et al., Anesth. Analg. 101 (2005) 1401-1406; Chou et al., Am. J. Clin. Pathol. 124 (2005) 330-338.

The above described assays are known in the art, e.g. from standard text books such as Lottspeich, Engel “Bioanalytik” Spektrum Akademischer Verlag (2006); Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989); Higgins and Hames (Eds.) “Nucleic acid hybridization, a practical approach” IRL Press Oxford, Washington D.C., (1985); Nollau et al, Olin. Chem. 43 (1997), 1114-1128; The use of some of the recited assays is described in the appended examples.

The diagnostic composition may be particularly useful for determining expression of P2Y5 receptor and P2RY5 gene product in a biological sample. As the above described mutations within the P2RY5 gene result in the expression of truncated, non-functional P2Y5 receptor protein, decreased expression levels of full length protein as well as detection of mutations may be used to ascertain that an individual does not have a disease or disorder, or is at risk of developing diseases or disorders associated with aberrant P2Y5 receptor or P2RY5 expression, preferably diseases selected from those described herein above.

In an alternative embodiment the invention provides a method for testing for the presence of the mutant nucleic acid molecule or the mutant polypeptide of the invention comprising assaying a sample obtained from a subject for the presence of said mutant nucleic acid molecule or mutant polypeptide, wherein the presence of the mutant nucleic acid molecule or the mutant polypeptide of the invention is indicative for hair-loss.

This method of the invention is for use in the determination of the presence of a nucleic acid molecule that is indicative of hair-loss and, thus, serves for diagnosing a predisposition of individual patients for hair-loss. Methods for testing a sample for the presence of the mutant nucleic acid molecule of the invention include, but are not limited to, nucleic acid amplification, sequencing or hybridization assays, such as for example Northern and Southern blots as well as immunohistochemical assays as described herein above in connection with diagnostic methods

In another preferred embodiment of the method of the invention said sample is blood, serum, plasma, fetal tissue, saliva, urine, mucosal tissue, mucus, vaginal tissue, fetal tissue obtained from the vagina, skin, hair, hair follicle or another human tissue. Preferably, the sample is blood, serum, plasma, saliva, urine, mucosal tissue or mucus.

The invention also relates to a Kit comprising at least one of the mutant nucleic acid molecule, the vector containing the mutant nucleic acid molecule, the host, the mutant polypeptide, the antibody, aptamer and/or phage and/or the anti-sense oligo- or polynucleotide, the fragment of the nucleic acid molecule or the primer pair of the invention.

The various components of the kit may be packaged in one or more containers such as one or more vials. The vials may, in addition to the components, comprise preservatives or buffers for storage.

The Figures show:

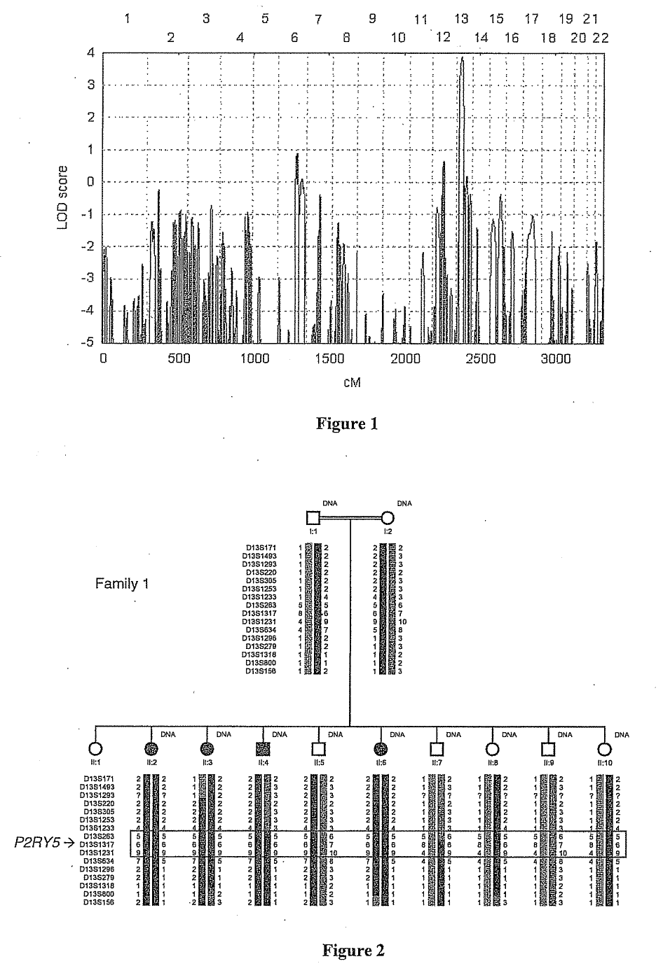

FIG. 1.

Multi-point LOD score for the linkage of Hypotrichosis to STR (short tandem repeat) markers covering chromosomes 1 to 22.

FIG. 2.

Pedigree of family 1, homozygosity mapping. Marker haplotypes on chromosome 13q that are linked to HS are indicated by black bars. Affected family members are shown in black, circles and squares denote females and males respectively. Microsatellite markers are given on the left, and the location of P2RY5 is indicated by an arrow. The homozygous region of the affected individuals is boxed, confined by a recombination in II:4 at the centromeric site and the observation of heterozygosity at the telomeric site.

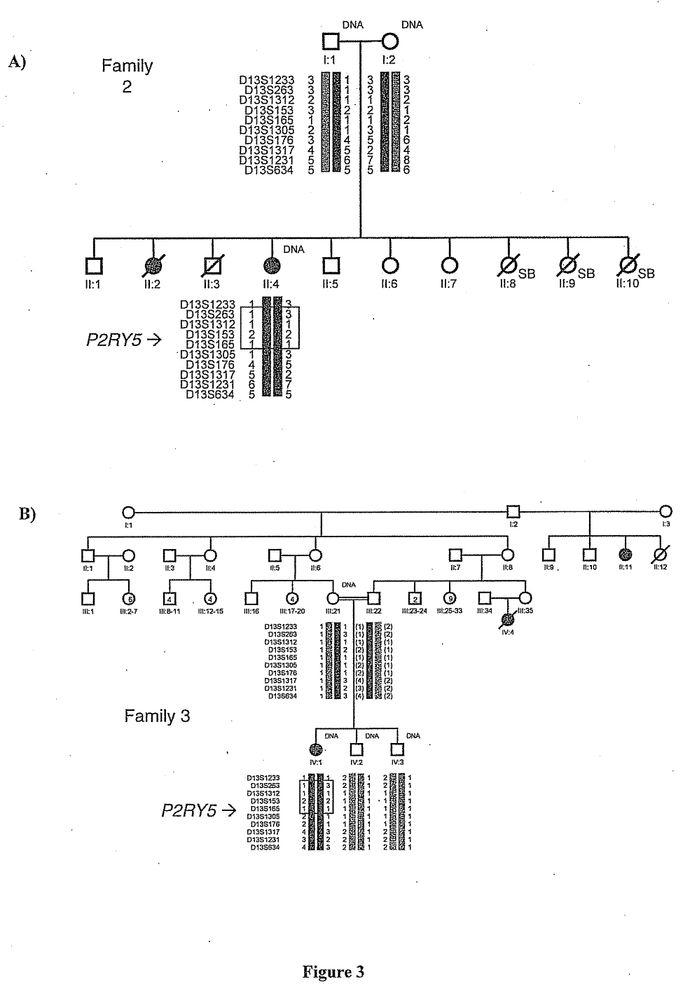

FIG. 3.

Pedigrees of HS-families 2 (A) and 3 (B) showing high resolution microsatellite genotyping in the region surrounding the P2RY5 gene. The chromosomal region carrying the disease mutation shared between the two families is boxed. This indicates a founder effect for the c.373_{—}374delAA mutation observed in families 2 and 3. SB, still birth.

FIG. 4.

Aligned sequences of mutation c.463C>T and a control (A) and of mutation c.373_{—}374delAA and a control (B).

FIG. 5.

Structure and biochemical analysis of P2Y5 wild-type and mutant proteins.

(A) Domain structure of wild-type and mutant proteins of P2Y5. The positions of the mutations c.463C>T and c.373_{—}374delAA are indicated by arrows.

(B) Western Blot analysis of COS7 cells transiently expressing wild-type and mutant P2Y5. The wild-type P2Y5 protein has a size of around 30 kDa, whereas the two truncated proteins have the anticipated sizes of 15 kDa and 17 kDa respectively. The proteins display additional bands, probably resulting from oligomerisation.

(C to E) Immunofluorescence analysis of COS7 cells transiently expressing wild-type and mutant P2Y5. Cells were stained with 4,6-diamidino-2-phenylindole-dihydrochloride (DAPI) and antibodies against V5 (P2Y5), and protein disulfide isomerase (PDI, mutant) or pan-cadherin (CAD, wild-type) respectively. P2Y5 wild-type is located in the cell membrane (C), the mutant P2Y5 proteins are located predominantly in the endoplasmic reticulum (D and E). Scale bars 10 μm.

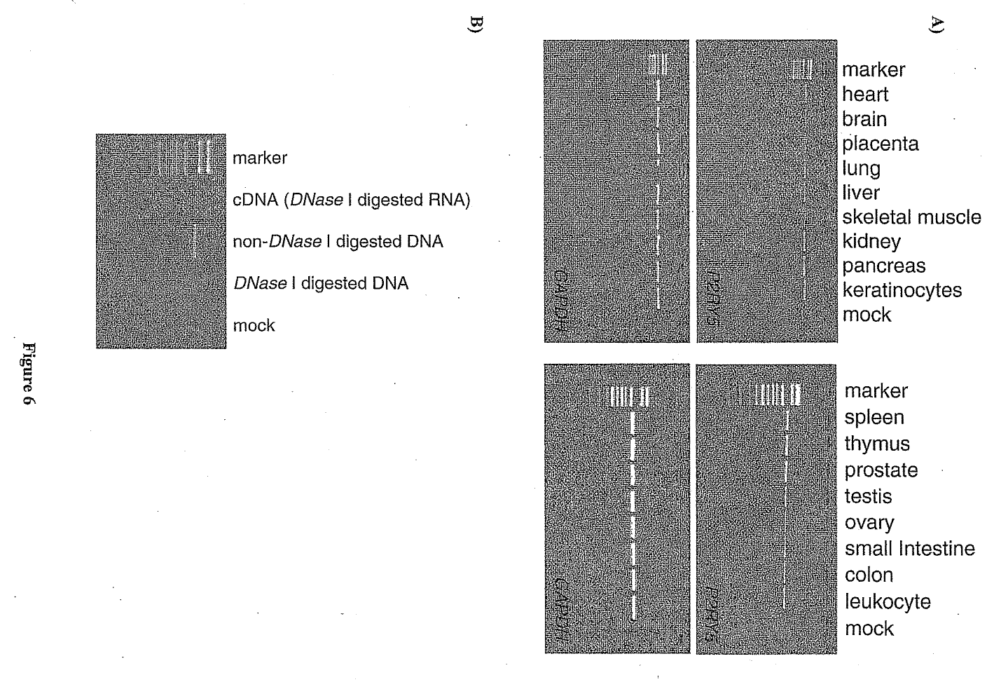

FIG. 6.

(A) Expression of P2RY5 and GAPDH in various normal human tissues and cells. RT-PCR analysis was performed using the Human MTC Panel I and II (BD Biosciences, Franklin Lakes, N.J.) and a human keratinocyte cDNA library (Clontech, Mountain View, Calif.). The last lane is a negative (no template) control. P2RY5 is expressed in all investigated tissues.

(B) DNA contamination of RNA-samples was prevented by DNase I digestion after isolation of RNA. Shown are PCR-products of P2RY5 obtained with primers P2RY5 1.1F and P2RY5 1R.

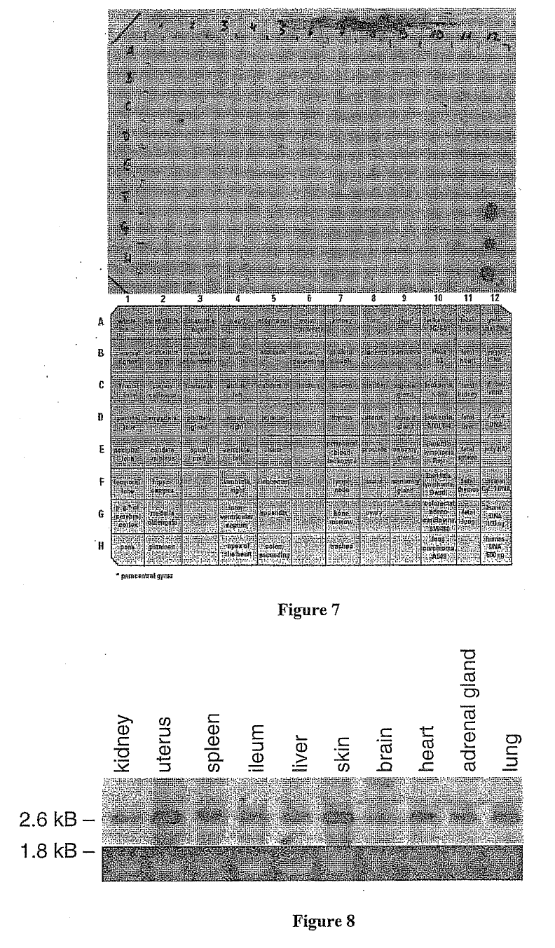

FIG. 7.

BD MTE Multiple Tissue Expression Human Array 3 probed with a P2Y5-probe.

FIG. 8.

Northern blot analysis of P2RY5 expression in mouse tissues. A DIG labelled cRNA probe corresponding to nucleotides 82 to 812 of mouse P2RY5 cDNA sequence was used for hybridisation. A single transcript of 2.4 kb is detected in all tissues. Reference markers of 2.6 kb and 1.8 kb are indicated. Below is shown an ethidium bromide stain of ribosomal RNA as a loading control.

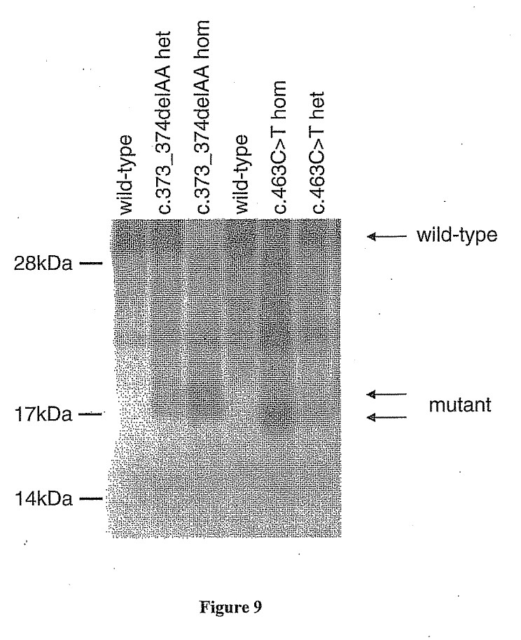

FIG. 9.

Protein truncation test (PTT) performed with the TNT® T7 Quick Coupled Transcription/Translation System (Promega, San Luis Obispo, Calif.) according to manufacturer's instructions. The PTT was performed with RNA from immortalized lymphocytes of a homozygous and a heterozygous member of family 3 carrying the c.373_{—}374AA deletion (FIG. 3B, III:21 and IV:1) and genomic DNA of a homozygous and a heterozygous member of family 1 carrying the c.463C>T transition (FIG. 2, II:2 and II:7) and two control individuals (wild-type).

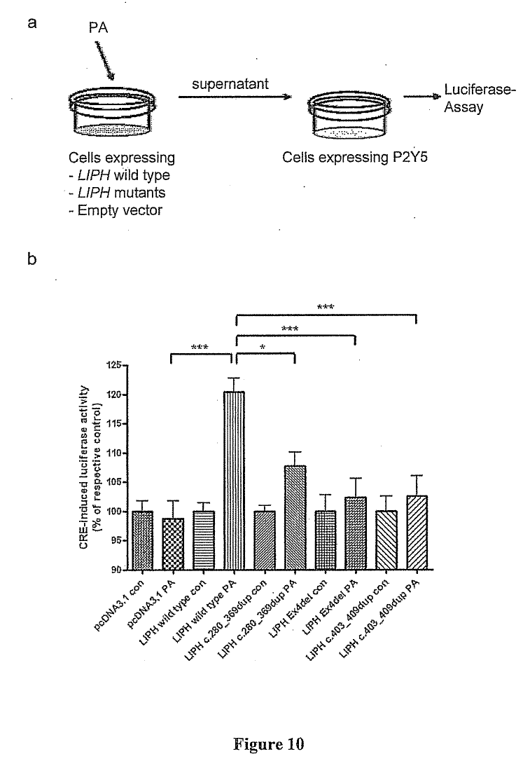

FIG. 10

Pharmacological analysis of LIPH wild type and mutants. (a) Schematic of experimental design. 100 μM phosphatidic acid (PA) or solvent (chloroform) was added for 30 minutes to Flp-In-CHO cells transiently expressing either LIPH wild type protein or a mutant LIPH protein (p.Gly94_Lys123dup, p.Gln137HisfsX1, p.G176_D209del). The supernatant was removed and added to cells expressing the P2Y5 receptor and a CRE-luciferase. Three hours later luciferase activity was assessed using the Bright-Glo™ Luciferase Assay System (Promega). (b) Effect of LIPH produced LPA on the cAMP response element (CRE)-directed luciferase activity in Flp-In-CHO cells transiently expressing P2Y5 and CRE-luciferase. The change in activity is given as percentage of the respective solvent control (con). Means±s.e.m. of 10 measurements. *, **, *** significant differences from the respective control (P<0.05, P<0.01 and P<0.001 respectively; ANOVA followed by the Bonferroni's multiple comparison test).

The Examples illustrate the invention:

### EXAMPLE 1

### Materials and Methods

### i) Linkage Analysis

Blood samples from eleven members of a consanguineous Saudi-Arabian family (K. Al Aboud, K. Al Hawsawi, D. Al Aboud, A. Al Githami, Clin Exp Dermatol 27, 654 (2002)) (the parents are third cousins, family 1, FIG. 2) were collected after obtaining informed consent. Samples from the two smaller families (D. Al Aboud, K. Al Aboud, K. Al Hawsawi, A. Al Aboud, Sudan J Dermatol 3, 128 (2005)) (families 2 and 3, FIG. 3)) were collected independently at a later date. Genomic DNA was extracted from families 1-3 according to standard methods. In addition, EBV- (Ebstein Barr Virus) transformed lymphoblastoid cell lines were created from samples of family 3 according to standard protocols (Current Protocols in Molecular Biology”, Volume 5, John Wiley and Sons, 2005).

A total genome scan was performed using 320 highly polymorphic microsatellite markers (Généthon) at an average density of 10-cM on an ABI 3100 Sequencer (Applied Biosystems, Foster City, Calif.). Mendelian incompatibilities were detected with the program PedCheck and erroneous genotypes deleted in the whole family (J. R. O'Connell, D. E. Weeks, Am J Hum Genet. 63, 259 (1998)). An autosomal-recessive model with full penetrance was assumed and a trait locus mutant allele frequency of 0.0001. The genetic marker map from Généthon with equal male and female recombination rates was used. Two-point LOD scores were calculated between each marker locus and HSS, with the LINKAGE version 5.21 software (G. M. Lathrop, J. M. Lalouel, C. Julier, J. Ott, Proc Natl Acad Sci USA 81, 3443 (1984)). For multi-point LOD score calculation and haplotyping Merlin and Simwalk2 were used (G. R. Abecasis, S. S. Cherny, W. O. Cookson, L. R. Cardon, Nat Genet. 30, 97 (2002); E. Sobel, K. Lange, Am J Hum Genet. 58, 1323 (1996)). The results of the multi-point LOD score calculation are shown in FIG. 1.

### ii) Mutation Screening

39 genes (including P2RY5, table 1) were screened by amplifying the coding regions and splice sites by PCR and direct sequencing of patient II:2 (family 1) and her parents.

PCR was performed in a total volume of 25 μl with 40 ng of genomic DNA using the REDTaq™ ReadyMix™ PCR Reaction Mix with MgCl₂ (Sigma-Aldrich, St. Louis, Mo.), under the following conditions: initial denaturation 95° C. 5 min, 1st cycle: 95° C. 30 s, 63° C. 30 s, 72° C. 30-80 s, 2nd cycle 95° C. 30 s, 60° C. 30 s, 72° C. 30-80 s, 3rd-34th cycle: 95° C. 30 s, 57° C. 30 s 72° C. 30-80 s, final extension 72° C. 7 min.

The PCR products were purified with the GFX PCR DNA Purification Kit (Amersham Biosciences, Buckinghamshire, UK) and were directly sequenced on an ABI 3100 genetic analyzer (Applied Biosystems, Foster City, Calif.) using the BigDye Terminator v1.1 Cycle Sequencing Kit (Applied Biosystems, Foster City, Calif.) and the DyeEx 2.0 Spin Kit (Qiagen, Hilden, Germany).

Primers for amplification of PCR-products for sequencing of P2RY5 and genotyping of microsatellites shown in FIG. 2 and FIG. 3 are given in tables 2, 3 and 4.

TABLE 1
Exclusion of positional candidate genes in chromosomal
region 13q14.11-13q21.33

| Gene abbreviation | Positional candidate gene |
|---|---|
| FOXO1A | Forkhead box protein O1A |
| ELF1 | E74-like factor 1(Ets domain transcription |
| | factor) |
| KBTBD6 | Kelch repeat and BTB (POZ) domain- |
| | containing 6 |
| KBTBD7 | Kelch repeat and BTB (POZ) domain |
| | containing 7 |
| RGC32 | Response gene to complement 32 |
| DGKH | Homo sapiens DGKH-1 mRNA for |
| | diacylglycerol kinase eta1 |
| AKAP11 | A-kinase anchor protein 11 |
| TNFSF11 | Tumor necrosis factor ligand superfamily |
| | member 11 |
| EPSTI1 | Epithelial stromal interaction 1 |
| DNAJC15 | DnaJ homolog subfamily C member 15 |
| ENOX1 | Ecto-NOX disulfide-thiol exchanger 1 |
| TSC22D1 | TSC22 domain family protein 1 |
| GTF2F2 | General transcription factor IIF, |
| | polypeptide 2 |
| KCTD4 | Potassium channel tetramerisation |
| | domain |
| TPT1 | Tumor protein, translationally-controlled 1 |
| weakly similar to | AK057244 |
| TRICHOHYALIN | |
| LCP1 | L-plastin |
| HTR2A | 5-hydroxytryptamine (serotonin) receptor |
| | 2A |
| MED4, DRIP36 | Mediator of RNA polymerase II |
| | transcription subunit 4 (Vitamin D3 |
| | receptor-interacting protein complex 36 kDa |
| | component) |
| RCBTB2 | Regulator of chromosome condensation |
| | and BTB |
| FNDC3A | Fibronectin type-III domain-containing |
| | protein 3a |
| MLNR | Motilin receptor (G protein-coupled |
| | receptor 38) |
| PHF11 | PHD finger protein 11 |
| ARL11 | ADP-ribosylation factor-like protein 11 |
| EBPL | Emopamil binding related protein, delta8-delta7 |
| TRIM13 | Tripartite motif-containing protein 13 (Ret |
| | finger protein 2) |
| KCNRG | Putative potassium channel regulatory |
| | protein |
| INTS6 | Integrator complex subunit 6 isoform b |
| NEK3 | NIMA-related kinase 3 |
| THSD1 | Thrombospondin type I domain-containing 1 |
| CKAP2 | Cytoskeleton-associated protein 2 |
| SUGT1 | Suppressor of G2 allele of SKP1 homolog |
| PCDH8 | Protocadherin-8 isoform 1 precursor |
| PCDH17 | Protocadherin-17 precursor |
| DIAPH3 | Diaphanous homolog 3 isoform a |
| PCDH20 | Protocadherin-20 precursor |
| PCDH9 | Protocadherin-9 precursor |

TABLE 2
Primers used for linkage analysis on chromosome 13.

| | | | | SEQ |
|---|---|---|---|---|
| Primer | | Fam | Fam | ID |
| denomination | sequence | 1 | 2 + 3 | NO: |
| D13S171F | 5′-CCTACCATTGACACTCTCAG-3′ | x | | 26 |
| D13S171R | 5′-TAGGGCCATCCATTCT-3′ | x | | 27 |
| D13S1493F | 5′-ACCTGTTGTATGGCAGCAGT-3′ | x | | 28 |
| D13S1493R | 5′-GGTTGACTCTTTCCCCAACT-3′ | x | | 29 |
| D13S1293F | 5′-TGCAGGTGGGAGTCAA-3′ | x | | 30 |
| D13S1293R | 5′-AAATAACAAGAAGTGACCTTCCTA-3′ | x | | 31 |
| D13S220F | 5′-CCAACATCGGGAACTG-3′ | x | | 32 |
| D13S220R | 5′-TGCATTCTTTAAGTCCATGTC-3′ | x | | 33 |
| D13S305F | 5′-TTGAGGACCTGTCGTTACG-3′ | x | | 34 |
| D13S305R | 5′-TTATAGAGCAGTTAAGGCACA-3′ | x | | 35 |
| D13S1253F | 5′-CCTGCATTTGTGTACGTGT-3′ | x | | 36 |
| D13S1253R | 5′-CAGAGCCGTGGTAGTATATTTTT-3′ | x | | 37 |
| D13S1233F | 5′-AGGACTANAGATGAATGCTC-3′ | x | x | 38 |
| D13S1233R | 5′-GACATGACTCCATGTTTGGT-3′ | x | x | 39 |
| D13S263F | 5′-CCTGGCCTGTTAGTTTTTATTGTTA-3′ | x | x | 40 |
| D13S263R | 5′-CCCAGTCTTGGGTATGTTTTTA-3′ | x | x | 41 |
| D13S1312F | 5′-TCTTCCCAGAATATATGGGA-3′ | | x | 64 |
| D13S1312R | 5′-AGCTGTAAAAGTGTTTGTTTGATGT-3′ | | x | 65 |
| D13S153F | 5′-AGCATTGTTTCATGTTGGTG-3′ | | x | 66 |
| D13S153R | 5′-CAGCAGTGAAGGTCTAAGCC-3′ | | x | 67 |
| D13S165F | 5′-GTTTCGCCAAGCCTGTT-3′ | | x | 68 |
| D13S165R | 5′-GTTGACAATAAAATACGCCACA-3′ | | x | 69 |
| D13S1305F | 5′-GATGGCACCATTGCAC-3′ | | x | 70 |
| D13S1305R | 5′-CAGCACATCCAAACAAGG-3′ | | x | 71 |
| D13S176F | 5′-CTGTGGGATTCCTTAGTGATAC-3′ | | x | 72 |
| D13S176R | 5′-ATATTCAGACAAAAGCCAAGTTA-3′ | | x | 73 |
| D13S1317F | 5′-CTTGGAAACCAACAAGTCAC-3′ | x | x | 42 |
| D13S1317R | 5′-ATTTTGCCACCTAGAACGG-3′ | x | x | 43 |
| D13S1231F | 5′-ACAGTTTTTCGAGGCCATATC-3′ | x | x | 44 |
| D13S1231R | 5′-GGTTAAATAAATCTCCATCCAGAAG-3′ | x | x | 45 |
| D13S634F | 5′-TCCAGATAGGCAGATTCAAT-3′ | x | x | 46 |
| D13S634R | 5′-CCTTCTTCTTCCCATTGATA-3′ | x | x | 47 |
| D13S1296F | 5′-TGCAGAAATGTGAGCC-3′ | x | | 48 |
| D13S1296R | 5′-TCCACCTAGAGCAACTACC-3′ | x | | 49 |
| D13S279F | 5′-TGGTTTGTTGCAGAAAGCACAC-3′ | x | | 50 |
| D13S279R | 5′-TTGGGCCTTGTCAACCTTCATA-3′ | x | | 51 |
| D13S1318F | 5′-GGCAAAGCCTTGCTCTTAAT-3′ | x | | 52 |
| D13S1318R | 5′-GGCTGTGCTCTTCCAAAATA-3′ | x | | 53 |
| D13S800F | 5′-AGGGATCTTCAGAGAAACAGG-3′ | x | | 54 |
| D13S800R | 5′-TGACACTATCAGCTCTCTGGC-3′ | x | | 55 |
| D13S156F | 5′-ATTAGCCCAGGTATGGTGAC-3′ | x | | 56 |
| D13S156R | 5′-GCTGTGGTATGAGTTACTTAAACAC-3′ | x | | 57 |

Fam 1 = Family 1; Fam 2 + 3 = Family 2 and 3

TABLE 3
Primers for amplification and sequencing of the
coding region of P2RY5

| | | SEQ |
|---|---|---|
| Primer | | ID |
| denomination | sequence | NO: |
| P2RY5 Ex1.1F | 5′-TGGAGGTTATAGAGGTTATAATC-3′ | 11 |
| P2RY5 Ex1.2F | 5′-TTATACCAACATGTACGGAAGC-3′ | 12 |
| P2RY5 Ext R | 5′-TGTTAATTTCTTTTGGAGGTGG-3′ | 13 |

TABLE 4
Primers for amplification and sequencing of
P2RY5 UTR

| | | SEQ |
|---|---|---|
| Primer | | ID |
| denomination | sequence | NO: |
| P2RY5 5′UTR 7F | 5′-TTGTGTGTCTTTAAGACCCTCC-3′ | 14 |
| P2RY5 5′UTR 7R | 5′-TCCTGCCTGGGCCGCAGAGC-3′ | 15 |
| P2RY5 5′UTR 6F | 5′-AAATGATACCGATAGACTTATGG-3′ | 16 |
| P2RY5 5′UTR 6R | 5′-TCTTTGATGACACCTATGAATAGC-3′ | 17 |
| P2RY5 5′UTR 5F | 5′-TTATAGGTGTGAACCACAGATCC-3′ | 18 |
| P2RY5 5′UTR 5R | 5′-TGTTGCCATAATGACCTTGTAG-3′ | 19 |
| P2RY5 5′UTR 4F | 5′-TGGGGTTTCTGTGTGGAAGTCC-3′ | 20 |
| P2RY5 5′UTR 3R | 5′-TCTCTATTTCCAACTGAGGTACCC-3′ | 21 |
| P2RY5 5′UTR 2R | 5′-AATGGATAGATCACTATTTGTTG-3′ | 22 |
| P2RY5 5′UTR 1F | 5′-AGGAAGTGCAAACAAACTGGG-3′ | 23 |
| P2RY5 5′UTR 1R | 5′-AGCAGTGGGAGCTGTTAACG-3′ | 24 |
| P2RY5 3′UTR R | 5′-AAAGGAATTCAAAGACATTACAG-3′ | 25 |

iii) Structure and Expression of Human P2RY5

Human occipital hair follicles from various individuals were obtained by plucking and were pooled and immediately stored in RNAlater™ Stabilization Solution (Ambion, Austin, Tex.). We isolated the RNA using the RNeasy Micro Kit (Qiagen, Hilden, Germany), followed by a RT-PCR using the SuperScript™ II RNase H⁻ Reverse Transcriptase (Invitrogen, Carlsbad, Calif.) and random hexamers under standard conditions (25° C. 10 min, 42° C. 50 min, 70° C. 15 min).

Since alternative transcripts of P2RY5 differing in the length of the 5′UTR are given in the database (UCSC, NCBI Build 36.1), we analysed the structure of P2RY5 expressed in hair follicle cells. Primers were created for different parts of the UTR (Table 5) and hair follicle cDNA was amplified by PCR using the REDTaq™ ReadyMix™ PCR Reaction Mix with MgCl₂ (Sigma-Aldrich, St. Louis, Mo.) under the following conditions: initial denaturation 95° C. 5 min, 1st cycle: 95° C. 30 s, 63° C. 30 s, 72° C. 80 s, 2nd cycle 95° C. 30 s, 60° C. 30 s, 72° C. 80 s, 3rd-37th cycle: 95° C. 30 s, 57° C. 30 s, 72° C. 80 s, final extension 72° C. 7 min. Samples were subjected to gel electrophoresis on 1.5% agarose gels. Only one transcript was found to be expressed in hair follicle cells consisting of the one coding exon, and resulting in a short 5′UTR. To determine the exact sequence of the 5′UTR, a RACE analysis with the 5′/3′ RACE Kit, 2^{nd} Generation (Roche Applied Science, Indianapolis, Ind.) was performed according to the manufacturer's instructions using the gene-specific primer 5′-AGCAGTGGGAGCTGTTAACG-3′ (SEQ ID NO:9). The sequence thus obtained is: 5′-NNTTTTTTTTTTTTTTTCACAGCAACAAATTTCATGTTTCTTTTTGG GTAIIICTGAGAAAAAGGAAATATTTATAAAACCATCCAAAGATCCAGATAATTT GCAAATAAATTGGAGGTTATAGAGGTTATAATCTGAATCCCAAAGGAGACTGCA GCTGATGAAAGTGCTTCCAAACTGAAAATTGGACGTGCCTTTACGATGGTAAG CGTTAACAGCTCCCACTGCT-3′ (SEQ ID NO:58).

TABLE 5
Primers for expression analysis of P2RY5 UTR in
human hair follicle cells (each forward-primer
was combined with the P2RY5 5′UTR1R Expr
reverse-primer).

| | | SEQ |
|---|---|---|
| Primer | | ID |
| denomination | sequence | NO: |
| P2RY5 5′UTR7F | 5′-TGGATTAGGAGAGCTTCTGG-3′ | 80 |
| Expr | | |
| P2RY5 5′UTR6F | 5′-TGGAAGTCACATTTCACCATGGC-3′ | 81 |
| Expr | | |
| P2RY5 5′UTR3F | 5′-TCTAAGGGTTGTGAAGATCACG-3′ | 82 |
| Expr | | |
| P2RY5 5′UTR2F | 5′-TCTTCCTCAGTAGAAACAACTGG-3′ | 83 |
| Expr | | |
| P2RY5 5′UTR1F | 5′-AAGATACAACTGGCAACTGAGG-3′ | 84 |
| Expr | | |
| P2RY5 5′UTR1R | 5′-AGCAGTGGGAGCTGTTAACG-3′ | 85 |
| Expr | | |

To study the expression of P2RY5-mRNA in different tissues, the Human MTC Panel I and II (BD Biosciences, Rockville, Md.) were used (FIG. 6A), as well as a human keratinocyte cDNA library (Clontech, Mountain View, Calif.). The cDNA was amplified by PCR using the P2RY5 Ex1.1F and the P2RY5 Ex1R primers (Table 3). As an internal standard the glyceraldehyde-3-phosphate dehydrogenase (GAPDH) was amplified using the primers 5′-AGCCACATCGCTCAGACACC-3′ (SEQ ID NO:62) and 5′-TCCTCTTGTGCTCTTGCTGGG-3′ (SEQ ID NO:63).

To prevent genomic DNA contamination, total RNA was treated with DNase I after isolation (FIG. 6B).

### iv) Northern Blot Analysis of Mouse P2RY5 in Various Tissues

Northern Blot analysis (FIG. 8) was performed using 14 μg of mouse total RNA prepared from tissues using RNeasy kits (Qiagen, Hilden, Germany). Samples were subjected to gel electrophoresis on 1.2% agarose gels in the presence of 0.7% formaldehyde. The RNA was transferred to a positively charged Nylon-membrane (Roche Applied Science, Indianapolis, Ind.) by vacuum blotting using a Model 785 Vacuum Blotter (BIORAD, Hercules, Calif.). Hybridisation was carried out at 68° C. in Dig Easy Hyb (Roche Applied Science, Indianapolis, Ind.). Stringency washes were performed at RT in 2×SSC/1% SDS and at 68° C. in 0.2×SSC/0.1% SDS. Bound digoxygenin (DIG) was visualised using alkaline phosphatase-conjugated anti-DIG antibody and CPD Star according to the manufacturer's recommendations (Roche Applied Science, Indianapolis, Ind.).

A 730 by cDNA fragment corresponding to nucleotides 82 to 812 of mouse P2RY5 cDNA sequence (mRNA, gi|31341175) was amplified by PCR using primers 5′-CTGGGAAGGCTTTCTTGCTCACTTCAG-3′ (SEQ ID NO:74) and 5′-GTTGGATATCAGCCCAAGCACGAAGAC-3′ (SEQ ID NO:75) from a mouse skin cDNA preparation. The PCR product was re-amplified using the forward primer and a modified reverse primer to which the T7 RNA polymerase binding site sequence GTAATACGACTCACTATAGGG had been added to the 5′ end. The identity of the PCR product was verified by sequencing. DIG-labelled cRNA probes were synthesized from the PCR products with T7 RNA polymerase in the presence of DIG RNA labeling mix (Roche Applied Science, Indianapolis, Ind.). Ethidium bromide stained ribosomal RNA was used as loading control.

### v) Cloning, Cell Culture and Protein Analysis

Taq polymerase-amplified PCR products (P2RY5 mutations and wild-type-sequence) were cloned in a eukaryotic expression vector with the pcDNA3.1/V5-His TOPO TA Expression Kit (Invitrogen, Carlsbad, Calif.) using the forward primer 5′-CGAAGCTTCCGCCGCCATGGGTAAGCCTATCCCTAACCCTCTCCTCGGTCTCG ATTCTACGATGGTAAGCGTTAACAGCTCCC-3′ (SEQ ID NO:76) and the reverse primer 5′-GCCTCGAGTCAGGCAGCAGATTCATTGTC-3′ (wild-type; SEQ ID NO:77), 5′-GCCTCGAGCTAAACAAAAACGGCGGGTGC-3′ (c.463C>T; SEQ ID NO:78) and 5′-GCCTCGAGTTACCCTGAGAGTGGGTAGAC-3′ (c.373_{—}374delAA; SEQ ID NO:79), respectively.

After reproduction in host cells (E. coli) and isolation of the vector, COS7 cells were transfected transiently. COS7 cells were cultured in Dulbecco's Modified Eagle's Medium with 4.5 g/L glucose and L-glutamine (Cambrex, East Rutherford, N.J.) supplemented with 10% foetal calf serum (FCS), Penicillin/Streptomycin and Amphotericin B (PAA, Pasching, Austria) at 37° C. in an atmosphere of 5% CO₂/95% air.

For immunofluorescence analysis (FIG. 5C to E), COS7 cells were seeded on glass coverslips in 15-mm Petri dishes and kept in medium without antibiotics 24 h before transfection. Cells were transiently transfected using FuGENE HD transfection reagent (Roche Applied Science, Indianapolis, Ind.) according to manufacturer's instructions. Cells were fixed two days post transfection with ice-cold methanol:acetone (1:1) for 5 min at room temperature (RT) and permeabilized with 1% Triton X-100 in phosphate buffer saline (PBS) for 10 min at RT. After blocking with 3% BSA in PBS for 30 min at RT, we detected the P2Y5 proteins with a monoclonal antibody to V5 (Invitrogen, Carlsbad, Calif.) at a dilution of 1:500. We visualized the endoplasmic reticulum and the cell membrane through anti-PDI (Stressgen Biotechnologies, Victoria, BC, Canada) and anti-pan Cadherin (United States Biological Swampscott, Mass.) polyclonal antibodies respectively at a dilution of 1:250. After being washed in PBS, cells were incubated for 1 h at RT with Cy3-conjugated AffiniPure Goat Anti-Mouse IgG (Jackson Immuno Research, West Grove, Pa.) and Fluorescein Conjugated Affinity Purified Anti-Rabbit IgG (Rockland, Gilbertsville, Pa.) secondary antibodies in a dilution of 1:1000 respectively. Coverslips were thoroughly washed in PBS three times for 5 min before mounting in antifade mounting medium containing 4,6-diamidino-2-phenylindole-dihydrochloride (DAPI; Vector Laboratories, Burlingame, Calif.). Cells were imaged with a Zeiss Axioplan2 microscope with appropriate excitation and emission filter pairs. We processed the images using the CytoVision 2.81 software.

For Western Blot analysis (FIG. 5B), COS7 cells were split in 60-mm Petri dishes 24 h before transfection, and kept in medium without antibiotics. Cells were transiently transfected using Lipofectamine 2000 (Invitrogen, Carlsbad, Calif.) according to manufacturer's instructions. Cells were harvested two days post transfection and centrifuged at 16.100 g for 10 min at 4° C. We resuspended the pellet in an appropriate amount of loading buffer (2× NuPAGE LDS Sample Buffer; Invitrogen, Carlsbad, Calif.) and 1× Complete Mini Protease Inhibitor Cocktail (Roche Applied Science, Indianapolis, Ind.) and lysed the cells using an ultrasonic bath. Equal amounts of proteins, including sample reducing agent (Invitrogen, Carlsbad, Calif.), were boiled at 95° C. for 5 min and subjected to SDS-PAGE. After protein separation on 4-12% Bis-Tris gels at 200 V for 45 min, we blotted the proteins semi-wet onto a Invitrolon PVDF membrane (Invitrogen, Carlsbad, Calif.) for 2 h at 30 V. Western Blot analysis was carried out using the Western Breeze Kit (Invitrogen, Carlsbad, Calif.) according to manufacturer's instructions. For detection of the expressed proteins an anti-V5 antibody (Invitrogen, Carlsbad, Calif.) was used in a dilution of 1:5000. Membranes were then exposed to an X-ray film for 15-30 min.

To verify the results of the Western Blot analysis, a protein truncation test (PTT) was performed with the TNT® T7 Quick Coupled Transcription/Translation System (Promega, San Luis Obispo, Calif.). RNA isolated from immortalized lymphocytes of a homozygous and a heterozygous person carrying the c.373_{—}374delAA deletion in the P2RY5 gene (FIG. 3B, III:21 and IV:1) and a control probe were used. In addition, a PTT using genomic DNA of homozygous and heterozygous persons carrying the c.463C>T transition (FIG. 2, II:2 and II:7) was performed, since P2RY5 contains no introns (FIG. 7).

### vi) Pharmacological Analysis of the Activity of the P2Y5 Receptor

Experiments to identify compounds that activate the orphan P2Y5 receptor may be performed as follows.

A V5 epitope-tagged wild-type human P2Y5 receptor can be stably expressed in Flp-In-CHO cells (vector pcDNA5/FRT/V5-His; Invitrogen, Carlsbad, Calif.). The cells can be cultured in F12 medium in the presence of 10% FCS and hygromycin 500 μg/ml (Invitrogen, Carlsbad, Calif.). Control experiments should be performed in Flp-In-CHO cells stably expressing for example the human platelet P2Y12 receptor or the truncated P2Y5 protein p. Lys125AsnfsX37 (vector pcDNA5/FRT/V5-His).

A possible activation of the receptor and, thus, coupling of the receptor to changes in, for example, intracellular adenylate cyclase activity can be assessed by the cAMP response element (CRE)-directed luciferase reporter gene assay 48 h after transient transfection of the cells with a pCRE-luc vector (Stratagene, Amsterdam, Netherlands). The transient transfection is performed in Optimem medium (Invitrogen, Carlsbad, Calif.) containing 2% FCS using lipofectamine 2000 (Invitrogen, Carlsbad, Calif.) as transfection reagent. Cells are subsequently cultured on 24-well plates and treated with pertussis toxin 200 ng/ml (PTX; Sigma-Aldrich, St. Louis, Mo.) or its solvent 20 h before the experiment. At the start of the experiment the culture medium is replaced by Optimem medium without serum. Drugs or their solvents (water or 2-propanol or dimethylsulfoxide, DMSO) are added for 3 h (all drugs and their solvents were obtained from Sigma-Aldrich, St. Louis, Mo.). The reaction is stopped by addition of Bright-GLO luciferase assay solution (Promega, San Luis Obispo, Calif.) used for cell lysis and the analysis of luciferase activity. Relative light units are measured using a single photon luminometer (Berthold, Wildbad, Germany). The luciferase activity determined for each well with cells is calculated as percentage of control (average activity in cells treated with solvent only). For statistical comparison one-way analysis of variance (ANOVA) followed by the Dunnett's post test is performed using GraphPad Prism version 4.03 (GraphPad, San Diego, Calif.).

Radioligand binding studies using [³H]-labelled test compounds may also be performed on HEK Flp-In cells (Invitrogen, Carlsbad, Calif.) stably expressing the P2Y5 receptor (wild-type) or the two truncated proteins (p.Gln155X and p.Lys125AsnfsX37; vector pcDNA5/FRT/V5-His; for culture media see above). For control experiments HEK Flp-In cells transfected with the empty vector are used. The binding assay is performed on intact cells cultured on 12-well plates coated with poly-L-lysine (Sigma-Aldrich, St. Louis, Mo.) as described previously (G. J. Molderings et al, in press (doi:10.1016/j.neuint.2007.04.022)). After washing of the cells with PBS containing bovine serum albumin (0.25% Sigma-Aldrich, St. Louis, Mo.), the cells are incubated for 30 min at 4° C. with 10 nM [³H]-labelled compound in the presence of the solvent used for the compound or in the presence of 50 μM not-labelled compound. The incubation is stopped by washing the cells with cold PBS (three times 0.5 ml). After cell lysis by NaOH 1 M and sodium dodecyl sulphate 0.1% (Sigma-Aldrich, St. Louis, Mo.), cellular radioactivity is measured by liquid scintillation counting. The values are normalized toward the protein contents of the cell lysates (dpm/mg protein). Specific binding of the [³H]-labelled compound is assessed by the differences in radioactivity measured in cells in the absence and in the presence of not-labelled compound. For statistical comparison one-way analysis of variance (ANOVA) followed by the Dunnett's post test is performed.

### EXAMPLE 2

### Identification of Mutations in P2Y5

We performed a genome-wide linkage analysis using 320 highly polymorphic microsatellite markers (as described above), having first excluded a number of candidate loci in this family (table 7). Homozygosity mapping revealed a new gene locus for HS on chromosome 13q14.11-13q21.33, with a maximum LOD score of 3.9 (θ=0.0) between recombinant markers D13S1233 and D13S634 (FIG. 2 and FIG. 1). Haplotype analysis defined a critical interval of around 28 Mb based on the smallest homozygous haplotype segment shared by the affected siblings (FIG. 2).

TABLE 7
Initial exclusion of candidate loci in family 1

| | Chromosomal | |
|---|---|---|
| Disease gene | location | Literature |
| Corneodesmosin | 6p21.3 | E. Levy-Nissenbaum et al., Nat Genet |
| (CDSN) | | 34, 151 (2003) |
| Hairless (HR) | 8p21 | W. Ahmad et al., Science 279, 720 |
| | | (1998); S. Cichon et al., Hum Mol Genet |
| | | 7, 1671 (1998) |
| Winged-helix-nude | 17q11.2 | J. Frank et al., Nature 398, 473 (1999) |
| (WHN) | | |
| Cadherin 3 (CDH3) | 16q22.1 | E. Sprecher et al., Nat Genet 29, 134 |
| | | (2001) |
| Gene not yet identified | 18p11 | A. Baumer et al., Eur J Hum Genet 8, |
| | | 443 (2000) |
| Vitamin D receptor | 12q13.11 | J. Miller et al., J Invest Dermatol 117, |
| (VDR) | | 612 (2001) |
| Keratin (KRT) gene | Chr. 12 and 17 | M. Hesse et al., Eur J Cell Biol 83, 19 |
| clusters | | (2004) |

The identified region of interest contains 61 known genes. After excluding 38 of these by direct sequencing (as described above; see Table 1), a nonsense mutation was identified in P2RY5 which encodes the orphan G protein-coupled receptor P2Y5. The four affected siblings carry a homozygous C>T transition (c.463C>T) which results in a premature termination of translation (p.Gln155X) (FIGS. 4A and 5A), both parents being heterozygous for the mutation. Sequencing of P2RY5 in two additional HS-families from Saudi-Arabia (families 2 and 3) revealed a two basepair deletion (c.373_{—}374delAA) leading to a frame-shift and a premature termination of translation (p.Lys125AsnfsX37) (FIGS. 4B and 5A) in both families. Haplotype analysis using densely spaced genetic markers suggests an ancestor common to both families (FIGS. 3A and B, Table 2). The families show a similar pattern of hair loss to family 1 and are described in detail elsewhere (D. Al Aboud, K. Al Aboud, K. Al Hawsawi, A. Al Aboud, Sudan J Dermatol 3, 128 (2005)). We did not detect either of the two mutations in 506 control chromosomes which included 138 chromosomes of Arabian origin.

### EXAMPLE 3

### Analysis of P2Y5

P2RY5 consists of one coding exon with a putative open reading frame of 344 amino acids (H. Herzog, K. Darby, Y. J. Hort, J. Shine, Genome Res 6, 858 (1996)). Expression analysis in human tissues (as described above) demonstrated that the P2RY5 mRNA is expressed in heart, brain, placenta, lung, liver, skeletal muscle, kidney, pancreas, spleen, thymus, prostate, testis, ovary, small intestine, colon and peripheral blood leukocytes (FIG. 6A).

Furthermore expression of P2Y5 was assessed using the BD MTE Multiple Tissue Expression Human Array 3. An almost ubiquitous expression of the mRNA was detected (FIG. 7). A listing of P2Y5-RNA tissue distribution is provided in Table 8.

TABLE 8
Tissue distribution of P2Y5 mRNA as determined using a
BD MTE Multiple Tissue Expression Human Array 3

| Tissue | Expression | Tissue | Expression |
|---|---|---|---|
| Whole Brain | + | Kidney | + |
| Cerebral cortex | + | Skeletal muscle | + |
| Frontal lobe | + | Spleen | + |
| Parietal lobe | + | Thymus | + |
| Occipital lobe | + | Peripheral blood | + |
| Temporal lobe | + | leukocyte | |
| Postcentral gyrus of cerebral | + | Lymph node | + |
| cortex | | trachea | + |
| Pons | + | Lung | + |
| Cerebellum, left | + | Placenta | + |
| Cerebellum, right | + | Bladder | + |
| Corpus callosum | + | Uterus | + |
| Amygdale | + | Prostate | + |
| Caudate nucleus | + | Testis | + |
| Hippocampus | + | Ovary | + |
| Medulla oblongata | + | Liver | + |
| Putamen | + | Pancreas | + |
| Substantia nigra | + | Adrenal gland | + |
| Accumbens nucleus | + | Thyroid gland | + |
| Thalamus | + | Salivary gland | + |
| Pituitary gland | + | Mammary gland | − |
| Spinal cord | + | Leukemia, HL-60 | − |
| Heart | + | HeLa S3 | + |
| Aorta | − | Leukemia, K-562 | + |
| Atrium, left | + | Leukemia, MOLT-4 | + |
| Atrium, right | + | Burkitt's lymphoma, Raji | + |
| Ventricle, left | + | Burkitt's lymphoma, | − |
| Ventricle, right | + | Daudi | |
| Interventricular septum | + | Colorectal adeno- | + |
| Apex of the heart | + | carcinoma, SW480 | |
| Esophagus | + | Lung carcinoma, A549 | − |
| Stomach | + | Fetal brain | + |
| Duodenum | + | Fetal heart | + |
| Jejunum | + | Fetal kidney | + |
| Ileum | + | Fetal liver | + |
| Ilocecum | + | Fetal spleen | + |
| Appendix | + | Fetal thymus | + |
| Colon, ascending | + | Fetal lung | + |
| Colon, transverse | + | Yeast total RNA | − |
| Colon, descending | − | Yeast tRNA | − |
| Rectum | − | E. coli rRNA | − |
| | | E. coli DNA | − |
| | | Poly r(A) | − |
| | | Human C₀t-1 DNA | + |
| | | Human DNA 100 ng | + |
| | | Human DNA 500 ng | + |

Western blotting of transiently transfected COS7 cell lysates (as described above) showed a signal at about 30 kDa, corresponding to the size anticipated for the wild-type protein. Both mutants also showed bands in their predicted size ranges (FIG. 5B, see also protein truncation test FIG. 9).

Subcellular localization of P2Y5 receptor proteins was studied using immunofluorescence analyses (as described above). We observed staining of the membrane for wild-type P2Y5 which co-localized with cadherins (FIG. 5C). In contrast, staining of the mutants revealed a network-like structure. Through co-staining for the protein disulfide isomerase (PDI), a marker for the endoplasmic reticulum (ER), we demonstrated the accumulation of truncated P2Y5 in the ER (FIGS. 5D and E).

### EXAMPLE 4

### Analysis of P2Y5 Activation Down-Stream of LIPH

### Reportergene-Assay

Flp-In-CHO cells of passages 19-23 were transiently transfected with different LIPH-constructs (LIPH wild type, p.Gly94_Lys123dup, p.Gln137HisfsX1, Ex4del). In addition, a second set of Flp-In-CHO cells were cotransfected with P2RY5 wild type sequence and cre-luciferase. For the control experiment, Flp-In-CHO cells transfected the empty pcDNA3.1 vector were used. Two days post transfection, cells were washed for three times with 37° C. warm Optimem (Invitrogen). Phosphatidic acid (100 μM PA, 3-sn-Phosphatidic acid sodium salt, Sigma) or its solvent (chloroform, Merck) was added to cells expressing LIPH-constructs for 30 minutes. The medium of the P2RY5 expressing cells was removed and replaced by 500 μl supernatant of the LIPH expressing cells after 30 minutes of PA- or solvent incubation. P2Y5 receptor function was assessed by the cAMP response element (CRE)-directed luciferase reporter gene assay after three hours as reported previously (Pasternack et al., 2008;) as shown in FIG. 10b. Relative light units were normalized against the respective solvent control for each LIPH construct.

### Results

It has recently been published that LPA is a ligand of the P2Y5 receptor and that the CRE-directed luciferase reporter gene assay is an adequate method to measure P2Y5-activity (Pasternack et al., 2008). It has also been observed that mutations in the LIPH gene cause hypotrichosis simplex. Thus, LIPH and P2Y5 may act in the same pathway that regulates hair growth and differentiation.

Dependent on this, we established and optimized an assay to indirectly measure LIPH activity. We therefore added PA or its solvent to LIPH expressing CHO cells and then offered the supernatant to cells expressing the P2Y5 receptor in addition to a CRE-luciferase. Afterwards, P2Y5 activity was measured by the CRE-directed luciferase reporter gene assay. Our data clearly show, that all examined LIPH mutations result in a significantly reduced activity of the encoded enzyme lipase H as shown by reduced CRE luciferase reporter gene activation.

1. A nucleic acid molecule, encoding a polypeptide having P2Y5 receptor function wherein said nucleic acid molecule comprises
a) a nucleic acid molecule encoding a polypeptide having the amino acid sequence of SEQ ID NO:2;
b) a nucleic acid molecule having the DNA sequence of SEQ ID NO:1;
c) a nucleic acid molecule having the sequence of SEQ ID NO:1, wherein each thymidine is replaced by uridine;
d) a nucleic acid molecule that hybridizes under stringent conditions to the complementary strand of a nucleic acid molecule of (a), (b) or (c);
e) a nucleic acid molecule encoding a polypeptide having at least 80% sequence identity to the polypeptide of (a); or
f) a nucleic acid molecule that is degenerate with respect to the nucleic acid molecule of (b), (c) or (d);
for the diagnosis, treatment and/or prevention of hair-loss and the diagnosis of a predisposition for hair-loss.

2. The nucleic acid molecule of claim 1, which is contained in a vector.

3. The nucleic acid molecule of claim 2, wherein the vector is contained in a host cell.

4. A polypeptide encoded by the nucleic acid molecule of claim 1 for the treatment and/or prevention of hair-loss.

5. A pharmaceutical composition comprising
i) a nucleic acid molecule encoding a polypeptide having P2Y5 receptor function wherein said nucleic acid molecule comprises
a) a nucleic acid molecule encoding a polypeptide having the amino acid sequence of SEQ ID NO:2;
b) a nucleic acid molecule having the DNA sequence of SEQ ID NO:1;
c) a nucleic acid molecule having the sequence of SEQ ID NO:1, wherein each thymidine is replaced by uridine;
d) a nucleic acid molecule that hybridizes under stringent conditions to the complementary strand of a nucleic acid molecule of (a), (b) or (c);
e) a nucleic acid molecule encoding a polypeptide having at least 80% sequence identity to the polypeptide of (a); or
f) a nucleic acid molecule that is degenerate with respect to the nucleic acid molecule of (b), (c) or (d);
ii) a vector comprising the nucleic acid molecule of (i);
iii) a host cell comprising the vector of (ii); or
iv) a polypeptide encoded by the nucleic acid molecule of (i).

6. A method of treating and/or preventing hair-loss comprising administering the pharmaceutical composition of claim 5 to a subject in need thereof.

7. A
i) nucleic acid molecule encoding a polypeptide having P2Y5 receptor function wherein said nucleic acid molecule comprises
a) a nucleic acid molecule encoding a polypeptide having the amino acid sequence of SEQ ID NO:2;
b) a nucleic acid molecule having the DNA sequence of SEQ ID NO:1;
c) a nucleic acid molecule having the sequence of SEQ ID NO:1, wherein each thymidine is replaced by uridine;
d) a nucleic acid molecule that hybridizes under stringent conditions to the complementary strand of a nucleic acid molecule of (a), (b) or (c);
e) a nucleic acid molecule encoding a polypeptide having at least 80% sequence identity to the polypeptide of (a); or
a nucleic acid molecule that is degenerate with respect to the nucleic acid molecule of (b), (c) or (d); or
ii) a vector comprising the nucleic acid molecule of (i); or
iii) a host cell comprising the vector of (ii); or
iv) a polypeptide encoded by the nucleic acid molecule of (i)
for use in treating and/or preventing hair-loss.

8. A method for the identification of a compound useful in the treatment of hair-loss or as a lead compound for the development of an agent for treating hair-loss comprising the steps:
i) determining the level of P2Y5 receptor protein or P2RY5 transcript in a cell wherein said cell comprises P2RY5 DNA in expressible form;
ii) contacting said cell with a test compound;
iii) determining the level of P2Y5 receptor protein or P2RY5 transcript in said cell after contacting with the test compound; and
iv) comparing the P2Y5 receptor protein or P2RY5 transcript level determined in step (iii) with the P2Y5 receptor protein or P2RY5 transcript level determined in step (i), wherein an increase of P2Y5 receptor protein or P2RY5 transcript level in step (iii) as compared to step (i) indicates that the test compound is a compound useful in the treatment of hair-loss or as a lead compound for the development of an agent for treating hair-loss.

9. The method according to claim 8 wherein said cell comprises a the nucleic acid molecule fused to a reporter gene, wherein the nucleic acid molecule comprises
a) a nucleic acid molecule encoding a polypeptide having the amino acid sequence of SEQ ID NO:2;
b) a nucleic acid molecule having the DNA sequence of SEQ ID NO:1;
c) a nucleic acid molecule having the sequence of SEQ ID NO:1, wherein each thymidine is replaced by uridine;
d) a nucleic acid molecule that hybridizes under stringent conditions to the complementary strand of a nucleic acid molecule of (a), (b) or (c);
e) a nucleic acid molecule encoding a polypeptide having at least 80% sequence identity to the polypeptide of (a); or
f) a nucleic acid molecule that is degenerate with respect to the nucleic acid molecule of (b), (c) or (d).

10. A method for the identification of a compound useful in the treatment of hair-loss or as a lead compound for the development of an agent for treating hair-loss comprising the steps:
i) contacting a cell containing P2Y5 receptor protein and a P2Y5 target molecule with a test compound; and
ii) determining the level of activity of the P2Y5 target molecule before contacting the P2Y5 protein with the test compound and after contacting the P2Y5 protein with the test compound, wherein an increased activity of the target molecule after contacting the P2Y5 protein with the test compound as compared to the level before contacting the P2Y5 protein with the test compound indicates that the test compound is a compound useful in the treatment of hair-loss or as a lead compound for the development of an agent for treating hair-loss.

11. A nucleic acid molecule deviating from the nucleic acid molecule of claim 1 by at least one mutation, wherein said mutation results in a loss of function of the polypeptide encoded by the nucleic acid molecule of claim 1 and is selected from:
i) a substitution;
ii) a deletion;
iii) an inversion; and/or
iv) an insertion;
and wherein said mutation is causative and/or indicative of hair-loss.

12. The nucleic acid molecule of claim 11, wherein said substitution is a cytosine to thymidine exchange at a nucleotide position corresponding to position 463 of the nucleotide sequence of SEQ ID NO:1.

13. The nucleic acid molecule of claim 11 wherein said mutation is a cytosine to thymidine exchange at a nucleotide position corresponding to position 463 of the nucleotide sequence of SEQ ID NO:1, and/or a deletion of the adenosine at a nucleotide position corresponding to position 373 of the nucleotide sequence of SEQ ID NO:1, and/or a deletion of the adenosine at a nucleotide position corresponding to position 374 of the nucleotide sequence of SEQ ID NO:1.

14. A vector comprising the nucleic acid molecule according to any claim 11.

15. A host transformed with the vector of claim 14.

16. A method of producing a polypeptide comprising culturing the host of claim 15 under suitable conditions and isolating the polypeptide produced.

17. A polypeptide encoded by the nucleic acid molecule according to claim 11.

18. An antibody, aptamer or phage that specifically binds to the nucleic acid molecule according to claim 11 or a polypeptide encoded by the nucleic acid molecule according to claim 11.

19. An oligo- or polynucleotide comprising or consisting of an oligo- or polynucleotide selected from the group consisting of:
(a) an oligo- or polynucleotide consisting of at least 10 consecutive nucleotides of SEQ ID NO:5 or 7; wherein the at least 10 consecutive nucleotides contain a T at position 463 of SEQ ID NO:5, or a G at position 373 of SEQ ID NO:7;
(b) an oligo- or polynucleotide hybridizing under stringent conditions to at least a portion of the oligo- or polynucleotide of (a), wherein said portion comprises the nucleotide in position 463 of SEQ ID NOs:5 or the nucleotide in position 373 of SEQ ID NO:7, wherein said oligo- or polynucleotide contains a T at position 463 of SEQ ID NO:5 or a G at position 373 of SEQ ID NO:7; and
(c) an oligo- or polynucleotide identical to the oligo- or polynucleotide of (a) or (b) with the exception that T is replaced by U.

20. A diagnostic composition comprising the nucleic acid molecule according to claim 1 or a polypeptide encoded by the nucleic acid molecule of claim 1.

21. A method for testing for the presence of the nucleic acid molecule according to claim 11 the a polypeptide encoded by the nucleic acid molecule of claim 11, the method comprising assaying a sample obtained from a subject for the presence of said nucleic acid molecule or polypeptide, wherein the presence of the nucleic acid molecule according to claim 11 or polypeptide encoded by the nucleic acid molecule of claim 11 of is indicative for hair-loss.

22. The method of claim 21, wherein said sample is blood, serum, plasma, saliva, urine, mucosal tissue or mucus.

23. A kit comprising at least one of
a) the nucleic acid molecule according to claim 11;
b) a vector comprising the nucleic acid molecule according to claim 11;
c) a host transformed with a vector comprising the nucleic acid molecule according to claim 11;
d) a polypeptide encoded by the nucleic acid molecule according to claim 11; and/or
e) an antibody, aptamer or phage that specifically binds to the nucleic acid molecule according to claim 11 or a polypeptide encoded by the nucleic acid molecule according to claim 11;
f).

24. The method according to claim 6, wherein the hair-loss is selected from the group consisting of hypotrichosis simplex of the scalp (HSS), hypotrichosis simplex, generalised form (HSG)), alopecia universalis congenitalis, papular atrichia, hypotrichosis Marie Unna and monilethrix.

25. A diagnostic composition comprising the nucleic acid molecule according to claim 11, a polypeptide encoded by the nucleic acid molecule of claim 11, or an antibody, aptamer or phage that specifically binds to said nucleic acid molecule or polypeptide.

26. A diagnostic composition comprising the oligo- or polynucleotide according to claim 19.

27. A kit comprising the oligo- or polynucleotide of claim 19.


SEQUENCE LISTING (97 sequences)

SEQ ID NO: 1 - DNA, 1035 bp, homo sapiens
atggtaagcgttaacagctcccactgcttctataatgactcctttaagtacactttgtatgggtgcatgttcagcatggtgtttgtgcttgggttaatatccaattgtgttgccatatacattttcatctgcgtcctcaaagtccgaaatgaaactacaacttacatgattaacttggcaatgtcagacttgctttttgtttttactttacccttcaggattttttacttcacaacacggaattggccatttggagatttactttgtaagatttctgtgatgctgttttataccaacatgtacggaagcattctgttcttaacctgtattagtgtagatcgatttctggcaattgtctacccatttaagtcaaagactctaagaaccaaaagaaatgcaaagattgtttgcactggcgtgtggttaactgtgatcggaggaagtgcacccgccgtttttgttcagtctacccactctcagggtaacaatgcctcagaagcctgctttgaaaattttccagaagccacatggaaaacatatctctcaaggattgtaattttcatcgaaatagtgggattttttattcctctaattttaaatgtaacttgttctagtatggtgctaaaaactttaaccaaacctgttacattaagtagaagcaaaataaacaaaactaaggttttaaaaatgatttttgtacatttgatcatattctgtttctgttttgttccttacaatatcaatcttattttatattctcttgtgagaacacaaacatttgttaattgctcagtagtggcagcagtaaggacaatgtacccaatcactctctgtattgctgtttccaactgttgttttgaccctatagtttactactttacatcggacacaattcagaattcaataaaaatgaaaaactggtctgtcaggagaagtgacttcagattctctgaagttcatggtgcagagaattttattcagcataacctacagaccttaaaaagtaagatatttgacaatgaatctgctgcctga

SEQ ID NO: 2 - protein, 344 aa, homo sapiens
MVSVNSSHCFYNDSFKYTLYGCMFSMVFVLGLISNCVAIYIFICVLKVRNETTTYMINLAMSDLLFVFTLPFRIFYFTTRNWPFGDLLCKISVMLFYTNMYGSILFLTCISVDRFLAIVYPFKSKTLRTKRNAKIVCTGVWLTVIGGSAPAVFVQSTHSQGNNASEACFENFPEATWKTYLSRIVIFIEIVGFFIPLILNVTCSSMVLKTLTKPVTLSRSKINKTKVLKMIFVHLIIFCFCFVPYNINLILYSLVRTQTFVNCSVVAAVRTMYPITLCIAVSNCCFDPIVYYFTSDTIQNSIKMKNWSVRRSDFRFSEVHGAENFIQHNLQTLKSKIFDNESAA

SEQ ID NO: 3 - DNA, 3003 bp, homo sapiens
tggattaggagagcttctggatagctgaacacatggaggtttctggagggtgtggcccccagagggcatggaagctctgcacccctgctcccatacctcaccctacgcatctcctcatctgtatcctttgcaatatcctttgtaataaaccgaaaattttatcataatgagcatcaaaagaatcccaaatgagaaaactgctttttgcataagtgtaaattaagagatggaagtcacatttcaccatggcaaacatttgctgcatgcaaaatgactgaaacaaggtcttgatctgtcaccaggctgaagtgcagtggtacaatcaaagctcactgcagtctcaacctcctaggctcaaacaatcctcccacttcagcctcccaaccagctgggaccacaggttgacatcaccacacccaggtctgctggaatttgctggaggtccactccagaccctgtttgtctggatatcaccagcagaggttgcagaacaacaaagattgctgcctgttccttcctctggaagctttgtcccagaggggcacccaccagacggcagctggagctctcctgtgtgccactggagtacgaaaaagaaaaaatcctgcagctagctcggtgtctgcccaaacggctgcccagttttgtgcttgaaacccagggcctgggtggtgtaggcacccgagggaattgcctggtctaagggttgtgaagatcacggaaaaggcatagagtctgggccggaatgcatcgttcctcgtggcacattccctcatggcttcccttagctaggggaggaagttccctggccccttgcacttcccgttccctctgctatggctcttcctcagtagaaacaactggcaacaaaattcaagtttatgattcattcatcagcaaacatgtgagaatcatctacaaagaaccaagaattgtgagaaagcgacctcaagatacaactggcaactgaggaaaaggcctcaattcaacaagagctaacaagcttgggagtttatttcggaatctttaaaagactcttctgcttacccacaatctgggatccactgcaggaaaacaaaaaaggaaaacttcatttaaaagaagcaagaagtaaaatgggacaaattgggaatgtttaagtctctgaaactctgcactgaaaagaaaataagattgataacttaagcttaacattctgaggcataaagaaacattaactttggagtattcatcttgactactgaaatacaagtttagaagacaagtggtttcattctggtcacagatcacagcttttctttaaatttataatcctatgggttggactcgttgactgtattttttaaaggttgctcgtcagttaactgagccttggaattcatggattttctaaagactaacaaatgaaaatattttcctgttgaagaacccagcggaaattttacagcaacaaatttcatgtttcttttgggtatttctgagaaaaaggaaatatttataaaaccatccaaagatccagataatttgcaaataaattggaggttatagaggttataatctgaatcccaaaggagactgcagctgatgaaagtgcttccaaactgaaaattggacgtgcctttacgatggtaagcgttaacagctcccactgcttctataatgactcctttaagtacactttgtatgggtgcatgttcagcatggtgtttgtgcttgggttaatatccaattgtgttgccatatacattttcatctgcgtcctcaaagtccgaaatgaaactacaacttacatgattaacttggcaatgtcagacttgctttttgtttttactttacccttcaggattttttacttcacaacacggaattggccatttggagatttactttgtaagatttctgtgatgctgttttataccaacatgtacggaagcattctgttcttaacctgtattagtgtagatcgatttctggcaattgtctacccatttaagtcaaagactctaagaaccaaaagaaatgcaaagattgtttgcactggcgtgtggttaactgtgatcggaggaagtgcacccgccgtttttgttcagtctacccactctcagggtaacaatgcctcagaagcctgctttgaaaattttccagaagccacatggaaaacatatctctcaaggattgtaattttcatcgaaatagtgggattttttattcctctaattttaaatgtaacttgttctagtatggtgctaaaaactttaaccaaacctgttacattaagtagaagcaaaataaacaaaactaaggttttaaaaatgatttttgtacatttgatcatattctgtttctgttttgttccttacaatatcaatcttattttatattctcttgtgagaacacaaacatttgttaattgctcagtagtggcagcagtaaggacaatgtacccaatcactctctgtattgctgtttccaactgttgttttgaccctatagtttactactttacatcggacacaattcagaattcaataaaaatgaaaaactggtctgtcaggagaagtgacttcagattctctgaagttcatggtgcagagaattttattcagcataacctacagaccttaaaaagtaagatatttgacaatgaatctgctgcctgaaataaaaccattaggactcactgggacagaactttcaagttccttcaactgtgaaaagtgtctttttggacaaactatttttccacctccaaaagaaattaacacatggacattttaaagtctttagtataaagaaaatttgtattcaatgtgttaagcattaacatgtattttatttgtgtatccactccatctgatttttctgagccattttgatttgttccttcattaaaaaaaatctcttaaagttatttagtgtctaaaagtgactgacttaaattatgtggtgccaatctgtaatgtctttgaattcctttttatattaaatgatttaatttactaa

SEQ ID NO: 4 - DNA, 35659 bp, homo sapiens
agaaaaatcctatgaaagaacagggttctctcatgaagaataaacagggagatccaggtggatggggtgggttcaaggaaggctgatctgggaaggtaacatggacgctgagtcttgaaatgtgagaaggaggctgtgctttgaaaggagaaggggcagagatcttccaaaagatgaaagatcatatgctaagaaactgatgttctttcagaaaatgaaagaggccaccagaactaaagtacccgggtgggatagtggagaggttgctaaagaagaggttgctgtatgatcacgcagaatctgaaagaccatggtaaaacgtttaaatattctaagagaaacagtaaaccaattacaacagaaactctgttttaggataagtcactaatatagagatagctagttcaattgtgtctggcttcctatcacatcactagcacttagtacagaattggggtcctaaaaatatttggcaatgatgacctgtgttgctttcaggaaagtattccaagtgatagggtccaccataatccatattgctttagctctgttacaagtgacaaaattttctatcaatttccaacttcctagctccaatgtctcaagaatatgccaggcgggtgtaggcacagctccagacagcaaacaactttggggttgtatgcatataactcaggagttaactctattgatctgtgaaattaaaaaaaaaaaaactactatttactcaatttacttaagtctttaacttcatattatgcaaaactgaaaatatataaaaactgttgtgttgttataacgtttggtgtggtaggcctcaggggcaggcctcggaccttctcttgccctcctttaacctgttccaaggcaggactctaatttttcccccatcattgtaaattgtgtgtctttaagaccctccccagagatggtttttgccctatagtgtaggggaggaatgctgacatcataaaaatcccaatgctgacatcataaaaactggattaggagagcttctggatagctgaacacatggaggtttctggagggtgtggcccccagagggcatggaagctctgcacccctgctcccatacctcaccctacgcatctcctcatctgtatcctttgcaatatcctttgtaataaaccggtaagtgtgtttcctttgattttttctttttgttttttcagacagtcttgctctgcggcccaggcaggagtgcagtggcacaatctcagttcactgcaacctctgcctcctgggtttaggtgattctcgtgcctcagcctctggagtagctgggactacaggggcatgccaccacacccagataatttttgtatttttagtagagacggggttttgccatgttggccaggctgttcttgaactcctggcctcaagtgatctgcccacctcgacctcccaaagtgctgggattacaggcttgagccaccgtgcccagtatccttgagttttgtgagcagctccagcaaattaattgaacctgaagagtgtggtgtgagaaccccaatttgaagccagtaggtcagaagttctggaggcctaggatggtgtctgggggtatgaggtatgggcactgagaccccaaccagtgggatctgacactatctccaagtagacagtatcagaactgagttggaggatgcccagctggtatctgctgcttagtgtgtggggaaaaaaacccacacattttgtcacagaagtcttttgtgttgacaactgttgtggtggtatgagagcagaagaaaaacatggtttgagagagttttttcctacacatcactatgttctgggtttttatttcagcaatagcatgtcatcaacaaaaatgtttttcagcttatcttttttccaaaagaaaaaaaaatgtatataagcaatataaaacatagccaggtctaaaagcaacggaggtgcctaagatggaaaatttaaggaggcatttactctcacttgcacaatcttagtatgaaatgtacccttggtgccctgaacactaaactgacaataaaagctagttaatttaagatacactggcaaaatagaatgatgtgtcttttactttcatggtttccttacacaagaatgaaagtactttcagtttaaaaggttttatttgaatgtattacctcatttaatcttcaaaataagtcttaaaattaagtgttattattcattttatgaatcagaaaacttagctattttagtaaactgtgacttaatacattagaaatattttttgagtgtggaataacatttgaagtttatagatattaaaactaaaactcttttaaacttctttttaaaaataaaaaagcaaatgaatagagaagcttcctagaattaaatattaagatatttatgactctttctttaaaacattttattaatttcttatttttcagctttatccatttactgaaaaaggaatcttttttcttagctaaaaaatagctaagcattttttttaagtctttcaaaacagtaatcctttcgctaaattatagactaagaggagatatttttaagaacataacaatgtttaaaggaatagtaatgttatatcaatgacataattttatacacatgaacaaacacattttaaaatgtgtttcaaatggatttattaattaaatgtcttaaaatgtctttcaaagtggatttattaattgatatcttggtcaaaattttcaaagaaaatgagagatatatcaatttgattaggtctgatttcaaaaatttttattttattttaaactaagttactttgtggtgttgggtatttttttaaatatttaaagtattttgaaaagtgcaatatgtaaaatatattttgtgattgttctgttaattttaaaatattgtttaaagaaaggacttcctaactcaaaattagccaagatttctaaaattgggtccaggtttacttgaggatgtctgatataccagctctgggaggcagctccaagggagatgacaaaattcatgtaattaaactaaaatagtcacttatctgaatgaggtgttaacagaagtcaaagtcctcacccatcattactatcttcaattcagtgagggtcttcctcctttctaatagccaggaaaaaaaccttgttaaagagtttttgtggaatttatcctaagtacagtaggaaagttaccttctgaatccaaagaaaaaaatcctgttctaactgaaataagacaaaaatatctatgatgaatatttgcaaaatgaataaaaactcatactatatccatcctggctaacacggtgaaaccccatctctacaaaaaatacaaaaaattagccgggcatggttgcatgcgcctgtagtcccagctactcgggaggctgaggaaggagaatggcgtgaacccaggaggcagagcttgcagtgagtggagatggtgccactgcactccagcctgggcaacagagcaagactccatttcaaaaaaaaaaaataaaattcatactgtaggaagaattaatactataaaaggaactaatacaatgtgagaaacagacattaaaagaggaataggaaagttcttgaagctagcatgtctgaatatgatgaaagtcaaagatgcctcaccaaattttgtagatgtagaaggagtgggttacgactgaatagaaaaacacaagaagggtgaattcgagtttaaaagtcagaaataaacatgcttgtactgctaagtgacaaggcatcaaattatctattaataatgctcatgactaagatttgtgaataagttaaaaacaagtgaataaataggtcctataattaaatgatcaaaattaaagtcatttttaaataattcctgcctcttaactaggatctctataccacagaatgaaaatcaccaggtttttgccaatgggaatctaactggtaccctttacccatatatataattctatgatagcaaattgtgtcaagtttaacctattcctagtgctaaacttttttgttaaaaattgcttgatgattaaacacagtatagttttattttctagtaaataacacttaaaaattaaacaacactattaaaaattgtattggttttttgggttttgctgcttctacttctaggtggacctactccaagttcccagaatttcataatccttaaagcttccaattctgaaacagaactagatctaagacagtaggagtaggcttattagctagtattgggttcctaccaagtgatttttgttgatatgcacatgttactacttctatatgtttggaaacccgttggtcattcttgccctagtgccttaaaatcaaaggttggccagatacaatagaaatttgatatgtaaaggtaacataagggtcttcaggcacaatttagtttcaacacatccctcatgaaaaggatgattagtacccttataaaagggaccccagagagctgccttgtcccttccaccatctgaggacatggtaagaaggtgccatctatgaaccaggaaacaggcctccgccagatatcagatctgctggaaacttgatcttggacttcccagctttcagaactgtgagaaatacatttctgttgtttataagccaccaaatctatggtattttgttaaagcagactgaacagatccagtgtaataagagttcagaaaatctaatgattaatttcaagcttagactatcaatggcctaggtcatccttagaatgtctgcaggattctttttttcttccctacattttaattataagcaaaccttgaatattacaatatagggcattcttagcaatacagatgagagtttaaaatcataattttaaaattgttttctctgataactatgtgttcaatttgctttttaagctcctttcattttctatttattatgaatacattaattttttaaatgaaaagttaagcaagtataagagatgtctcttttttaccttttaacaaagaaaaccctttatctatgagtgcaactacaattttaaatttaactaagttcttcttggttaaaacaaaatcatataattaatactgactctagcccttgatcataaattctttgacacagtagaagaaaggcaaggcagatcgccttaccccaatctttatatatatgaagccaaatactaaatttcaaccaaaaaattaaactttggatcatgtgcccatcaggctatgcatgtaaataacaaacattaaagttaaaaaatatgcattagctatttgtacataagtaaatgaaaattaacagggcccaacatagacaatatcatacataaaagaaagatgtcttcatcaactctagcctccctcctctttgcctcacagaacatagcaaaagttatctcccaaatttcaaccacttctatctcaggaaaaatagttaatattttgtagaacatctgactttagttttcggttatattttatactgtaaactcctatttcctgatatggcttgtaatttatttgggaggcagcatatatcagagtccccaaggaagctttattaaacatgcatgccaggtttcccttttactcccaagattttgatatacccaaaaagagagaatatctacatgtaagaaaagctcctaagcagattctaatgtaatcctactattctatctccttcctaccctattcctgatacttctctcatatacatataaaaatatttagacttatgtatatatgtatgcatgtatctatctaatctcctccaccacctccttaagaagtataaactatatttctgcatttttctaattctgatataaatttctgaaaagtgacttaatattctttcttaaatagcaatattgtgataacagttcaatttcttgtcttcttaggttcaaaaaggccttctttcaaaccttccccaaagcctattttctggaacactaaaaactcaggcaaatttgaggtctatcaaatgtaaaaagtacagactctactgtgaatctcagaaattctctaaatcataacacattcaatagctgacagaagacttttgtttctctttttcccagcatttgggctaaagtgatgatacggtaagaaggcagaatattctgaacatttcattaactttagtttatgaggattaatttttaactttatcccagactcagtgggcaaaaatgttgtgtggactggtgaggatattgcactataaggaagattaacaaccgtaatctttctatcatgcctgatttcgataactatgattaatgattcaaagtcattaccttatacttcatgattctattttatttctctcactacctcactaccaaaattctgtcttcatgattatcttaatttaataatagtactagttaatatttattgaatatctactatatgctaggttctatgctaaatattttacatatattatctcatttaatcttcaaaatacgtccataaagtagatatgtgtattttatagaaattataaattttgccccatattatatggctgataagtgatggagcagtgattcaaacccaagtctacctgtttctaaaacttgtgccgttagctgctatactagtatgggagtcacgttcctttgcttactgcctctttcactctttctttgcccattgtcatgcttttaaccatgtttctaggttgctgtttacccctacaattagttccactggccacaattccatctatcattcattcactgatccattcatctagagatgctaagagaaatagcacattcatagtagtacccactacatagagttgttgtgaaaataaaataacctatataaaaagtttggtatgatacatggcacatgataagcaatcaataattctaccctcaaagagcttaacagataacatttattgaatgccagacatagcaccaaccacattacttatattatttaattctcacaacaaactctagaaagaaggtaattattttttaaatgccattttataagtgaagaaaggtagacaaggagtaaattgtccagtcacatgaataagtggtggatccaggacacagttccaaactgtttgatttcagagcctttgttttgaacttttatgctatagtttacagtctaatgaaaaaaaaaacagataagtaaataaaaaatagcaatacaaagtgctaagtaccagaataaaagaatacacagggtgccatgaaaatcagaagagacaactaactcagattaggagcattagagaaaacttcttggaagagattatgcttgagttgaatcctaaagaaagaacaaattattttaagtaaaaagcagaagaatagtgtgctcagaagcaagagaagaaaaaataatatggccctggggaaacttcatgcagctaatctataagataagaaaaataatgcttactatagggctgttttgacaactaagttagataataaatgtaaagtgcttagaagggcacctggcacacaagcctttgagacagaaatttttctttccataatgctaaaatacagatttcatagcaaggaaatagcaaaatataaactcgctgaaggattttagacaggtgactaccagggttcagatgggactttcagggagttcttaaaataaaattccagtgccaagcatataatggacacttggtaaatatttctagaatattgacatatgatgcagccatatcatgagtcaaattaactacagcaaagcacaaacaagaaaactgataataggatttgttttctaattgacactaagtcataactatggcagccagcctccaagatcaccccaataatctctgacactgggtatttatatccttgtgcagacagttcctaaactgtaccagggttagtctgtgggccaatgacatagggcagaagtgatggcatgtcacttctgagatgaatttataaaaatctgcagtttccatcttaggttctctccctctctaggatctcttccactggggttgaggcagggagctgctatggagtagccctacggagaggctcacatggcaaaaaaaatgaagcccctggccatcagccagcaaagaactgaggcctgccaacaaccacacgagtgaacttggaagtagactctccaatcccagtgaagccttaaactagctgcagccttggttgacaatttaagaatctctcatgaggaaacatgagtcaaaatcacacagtaaagccactcccagtttcccgactttcagaatttgtgtgagataataaatgttagttgttctaacagataattaatatagcaattgatcttttttaactataaagaaaatatttcatggaatacttaacttttgaaagtaaaatatgtatatttctttctaaagcattgtttctatccaggacttgggcagtagttttctcttgtactcaactcatgttattacttatctactcaaattaactgtcagcctaattgctctctgaacaacgcttcactctgttttgctacttagtcttgtgaaggaaaccaggctctcactgtcagcaagattagtataaatttcactgggtaaataatattgatagaaaaagtagaagctataagtctgttctacatgacttttcataactaacatacatttctaaggcaagcatgaacttgtctagaaagtaaaggactccaaggctattaagtcaaggtgctcctaaaagagattctgattatcaaagtaagtacaagtcagtagtgctagtagaaaaattaacaatagaggaagtggaaggcagaaattgctattgttttctgggtattttaaggaatattattagaactacattacaggaaatgtttttgtgccaccctaagctaaattctaagactgttttgcctcgcaagaggaaactagaatagaaaatagaagaattttggcagaccttggcctttctgaagatcttttccattttaccttgctggtcctttaatactaatatttctgcagatctgcttgccaatacatatgctggctgagccactatcatgatttttttttttaagagagagtcttgctcttgtcacccaggctggagtgcagtggtgcaatcttggctcactgcaacctccgtctcccgggttcaagcgattctcctgcctcagcctcccgagtagctgggattacaggcatgtgccaccatactcagctaatttttgtatttttagtagagatgaggtttcaccatgttggccaggctggtctcgaacttctgaccgcaggtgatccgcctgccttggcctctcaaagtgctgagattacaagcatgagccacagcaattggcctcatgattttaactatatctatattattcctaagtctacatttctgactaaaactatgctttcaagttctagatccatatttccaactggacaccaaagatatccacatgaatatgtggcaaacatctcaaatataataggttcaaacattaacatgaactcatttccttcatcaccaaccagctatgagatggtctttcaatacctactttggttaattgtgttatcaaccacctaatcacccatagtacaaatcttaaaattatcctcagttaacctcagttccatccaggacctaatcttttcattcatccttttaacatattcgtgtctttatatttacaatagatttcttttagatagcatagagttgggttttgtttttcatccactctgataatctctgccttttaattgggctgcttagagcactgacatttaatgcaattattaatataattagtttaaattctacatctatttgtctcatcttttctttgttccctttttgacaaagatacaaaagcaaatcaatagaaaaaggatagcctttaaaataaatggtgctgaaacaactgaacatccatggtgcccaactcagcaccccctaaaaaaacctcaacccaaatctcaaactttgtacaaaaataaacttaaaatggatcatggacttaaaatgtgaaactataattttttaagtataagggaaaatcttcaggatctggagttcttagatttgacaccaaaagcataatccataaaaggaaaaagtgataaactggactttatgaaaattaaaaacttttggatgagaaacatgatgtgaagaagatgaagaaagaagctacaggccaagagaaaatatttgctaaccacatatctaacaaaaaagactagtattgagaacatataaagaaccctcaaaactcaatggtaaaaaacaaacaatccaatcagaaaatgagcaaagacatgaagagaaatttcaccagagaggaaatacaggtcacaaataaacagggaaaggttaatcaatatcattagctatcatcctgatgggaaatgcaaattataacctcactgagatatccttacactatcagaatgactaaaataaaaaataaagacatcaccaaatgatggaaagaatgcagagaaactggatcactcactcgtatattgctggcggaaatttaaaatgattcaaccactctggaaaatacttttgcagtcttttatcaaagaaaatgcaaaataaccatacaactcttcaattttactcattaggcatttatcccagagaaaagaaaacccatactgatactaaacataaatgttcatagcagctttatttgtgatagccaaaaactagaacctgtcctgatattcttcaacatgtgtctggccaaagaaactgtagtacattcatgctacaaaatactagtcagcaataaaaaggtatgaactattgatacatgcaatatcgtgaatggatctccagaaaattatactgaatgagaaaaaaaactaatcctgtttgattccacttacataacatttttgaaatgacaaaatttgagaattgaagggcagattagcagtggccaggtgttagggacaggggtagagaggctggagggatatgggtgcaattgcttgtggtggtggatacatggacctacacattttcaccaggtcaagctgcctggtggaatggtgtatagtggaaacccctgaatagtgtctctctacaggcctggtgttcctgggctgagaaagagtctcggctgtagggttaaagagacttcctgggccagggtgttttttgtggctgggtgccttctgctggtttcacttgcttgcctggagcttcctaatggggaagggaatcttaggagtcttaggcttgttggataaaagcacctgaagaaaaatgactaatgctatttccagatattgctgacagagcaatattgaaataatctatctgttaaaattttttttctccgtttttattatcaagactgaatccaaaaacagattcactgaagaataattttatttatttatttattaaatggagagagattctcactatgttgtccaggccgttctcaaactactggcctcaactgatcctcctgcctcagcctcccaaagtgtaaggattacaggcatgagccaccaggcctggctcattgaagaagaattttaaaacttcaatgtttcaatttataatataataaaatacaaagctattatgattggagaaattatttattttaattctcaacattttaagaacatatattcttgtaacctttaaataaatattgaggccaggcacagtggctcatgcctataatcccagcactttgggaggctaaggcaggagaataacttgagtccaggagttcaagaccagcctgggcaacatagcaagaccctatctgaaaaaaagtatatatgtatatacatataaataaaacatattgaggtaataaagacaaaagttgtaagatatgcttcttctcaagaagaaactaattcaaagatgatgaatggaataaatctgaataagccaaagtccttatttgtctagtttaactgaaagttttccaaaagatatcattgttttgtaatacaatgcatacctactaaaggtaaaaaaaaaaaaatgcatttttgttaacagtaaaacaaataaaacagtggaacagttttatcaagaaacaattatgcttttgtcataatatttggtgtaagttataaaaaatggtttcatttaaatacagtctatcctatgtggcatacttatttaagacttatttagtggcagaaattctctgaacaaaaattttttacagattcatctccagcaggtactctgagggagtccctgaaaaagctcttttaagttacaaagcaaggcaacagaatcccaattctcttcttcaaatagtatacatcttaaataagaattttaatgcttctaatcaaagataattttaaaatttagagttagtggtatatttaaataattttaaatgttttaaaacatcaaagtacattttcctatgttctatatttacccaggtatactcataaatagaaaagtacatatgtatctttttgaaaaaaaaattaacgtctaaaagaatctatttctaataagtcacgttttagatatttcataatactgaggtttagtagcttacatgtatgttcatatcagaaattaatttatttctcatattgtttaaaatgtacagtggttaaaatcactttgagaaacttatggtatgacagagtttataagacacagtagaaagagttcttatctaagagcacataaaaagaaagagcagtatcccattccaaatcttaaagatattattctctgatttcaactatataagcataatagcaaagactgaaattaaatctggaaaatcataaacagcagatttattagagttgtagggttatgctatcttttttccttttaattttattctgtctcttataattgcttttcaagagccttaatagtaatatctctcctatattataaattaaaattttattccaaggaaataaaattttatattcctgtacgcttactcaagcatagttaggacaaataattgacagaagtcaaggaagacagttgacataggctgtcagatgtccctgctatcagggaaattaaattcaggagaagaaagttcagtctcttgaggtaaacaggatgctaaccaaagcattaaagagtaaaagacatgaacctaatcaccgaatggtgagaagagacaagtcagggtggaatgtgactgaaatggaactgttgatctggggctctttaatgagagacagctactctccgagaagtatttagtttcagaacactggcaagtagcccaaaaaaaaaaacaaaaaaacaaaacatgcattcctattttatgaggtgccatgaaaaataagaatcagaagcagaaatgtagaatcttaccaagttgaaatactgtttagaaatttctatttgccccaaagatcttggggacatgaacatggtgagtgaatttctgggaggtgagctggaaatacagattggagaaggccttttaaagatgttgatcagttaaaggttaattccctgagacaaccacatgattcattccttcacttctttcaagtctctgttcaaatgtccccttattagcaaggccttctttgacctccctatgtagaacagtaaaagcccaccccctttttcctcctaaattatttttcttcagagaacttatcaccatatgaccagttgtataattgtttatttattgattgtcttctccaaataaaatataagctcagggattttgttttattcactgctatattcccagcacctagaaaagcacctggtacacagttacactcaataaatattttgctactggaaaaaaaaataagtatcagttgacccagagatgaagacaacataagcaggaaaaaattttaatatgcctgtcttaatttcctatttatatggccagggcaaaaatccagcaatctttttcggttaggttgaaatagtccacttctgtttaactaaaacaacaacaaacggctgggctttaacagggggccttttgtagaagccgtcctttggtcttttgtttctaacctttctggagtagtgctcgtatcttctagtatagacaggaaaccctcaaattttccaaacatgtgaaaacagttgattaaagcaagcacactagaaaaccaataaaaaaaagtaaccacagaaattttgctctgggaatagatgactagtggcttagaatcataccaataattcttccttacctttgagtatcagtatctgttaaagagctagtctctttgaattccaagtgggttaagagcgaaacatttatctgcataaatgcctaaagtcaaaatatgtttttggttttaagcatacttgctttaatgttcatagcactagtccttaaaaatcaatcatctataataaaaagaaaagaaagaagtctaatcaaatgtatagtcatttttattttgaaggccaaaacttaaaaaaaatacaaatattaaatttgggtacctaaaactgagaaccacttttaagttaaggacaaaatgatatgcttgtttagatgcatctcccacctttttgaatgggttgttattttttgttagtttgttctaacagacttgtttccataatttgtcatttaactattctgtaaatgttataactcatatgattcatatgatggactgatgttttcattccactttatgttgttcttgcaacaaaattatttagtcaaacctatctttttttaaacactagattttatataatacttagaaagcctctactgctatttttaaatatattattttattttaattgttagagttgggctgtcacccaggctgaagtacagtggcatggcctactgcagccccaaactcctgggctccagaaatcaatccttctgcttcagcctctttgctgggattacaggtgtgagccacagcaggcctctactgttttttttttttgtttgtttgtttgtttttttgtttttgagatggagactcactctgtcacccaggctagagtgcagtggcacaatctcggctcactgcaacctccgccccctgggttcaagccattctcctgcctcagccttccgagtatctgggactacaggcacgtgccaccatgcccagctaatttttgtatttttagtagagacggggtttcaccacgttagctaggctggtgtcaaactcctgacctcagatgacccacctgtctcgcctcccaaagtgctgggattataggcatgagccacagtgcctggcctctactgttatttttctaacgtgctcatttttttccagaacttatatggagtcattttttaaatggagtcattttttattgagattattgatctagctagaatttattttggtataaagaataaatcaaagtctacctctttttttcaaattgctatcctgataactcatgagcatttactgaattatcctgaattttctccactgatttgaagtggataaattatcatacattaaattcccctgggtatgggattatttctggactccttataactaaagaaatgcaaactaaacaagatactactttgacctgttaaaagtctaataataatctggtttgggtgaaggtgtggagaatgcaaattaggaggggatttttttcttagaaatgaaactgctaggtgatataacccacagtatctacaggtgaacaaacttgtaaaaacatgcactgaaaacatactgagatttttactggattggcattaaatgtatagatttgtcccagaaaaactgcctggcacatgttcaagtattctcttatggcctttattaaagttttgtagtcttttgctttgttttcatctacatcctgcactttgcctattaagcttattcctaaggtattttgtactatttgttattgctgtaaattgaatatttttcattacattttccagttgattactgctggtacataaattgtcttatttgagagattggcaccagttaatttaaaaagctaagcaggaaatcattttaaaataatatatataattccatataacattttaattttaaaaacacaaaaagtgtattcatttgaaaatctctttacgtaaggtcaaggttttttgttttttttttagttctaagttgtttttcttctataaaccacaccaataatgtgggtttatgattttcactatgtgcagggtgaggacagaaaaatgcaaaggagtctgaggcctagagacacaatccttctactttccgacctgggtgggttcgcccctccttgggcaacctggtagtcccctgctcccaggaggtcaccatattgatgcagaacttagtgtggacacctaatcagcatagcgcacagcccagaactcctgggctccagtgatcttcccacctcagcctacagagtagctgggactacaggcatgtgtcactgcacctagcaatcctttctagtttcttacgttgccccctcatatcttttctacatctaagtctacaatattttctgcaacttgcctctgagtcatttcaaagcaccgaacttggtttgactaaaacaacccttttttgcacttccattttataacattacaataatatgaaaaatcagcaaatataaatcagggagtaaacaatgcttaaacttttaatacacacacaatttcactactgggagcaaaagaattcaggtatatcagagaatagctttatagccacaagcatgcagcttccaggagcatcctgagagaccaatcctttttaacttctacccctcagtaataggtaaaagatacaagtcaataagtgtttactttaactgaagagtacttggatttctgataataatctatgctggaaggtgttaaatttactattcacagaaaacccttgttctaattatttgaaagcagcagagatatttcctgcttttgtataaaacttggcacattactatgtacaaatgcaaactttttaaataaataaagatgaagctaaaaatgaaggatggtcatcatacgattttaatctacagaaggatgaaaatgtctaaaatctcttaatcttaaatttctatcataatgttagaatttagtgttttaaaaagtctgcagagcttactcatttaaagacaaagttctttgcttaatatttacctaagctatggaccaagtttcctattttatgtctgatatggtttcaatgtttgtcccttccaaatctcgtgttgaaatgtgatccccaatgctggaggtgaggtttggtagaagatgactcgtgttgaaatgtgctccccaatgctggaggtgaggtttggtagaagatgactcgtgttgaaatgtgctccccaatgctggaggtgaggtttggtagaagatgactcgtgttgaaatgtgctccccaatgctggaggtgaggtttggtagaagatgactcgtgttgaaatgtgctccccaatgctggaggtgaggtttggtagaagatgactcgtgttgaaatgtgctccccaatgctggaggtgaggtttggtagaagatgactcgtgttgaaatgtgctccccaatgctggaggtgaggtttggtagaagatgactcgtgttgaaatgtgctccccaatgctggaggtgaggtttggtagaagatgactcgtgttgaaatgtgctccccaatgctggaggtgaggtttggtagaagatgactcgtgttgaaatgtgctccccagtgctggaggtgaggtttggtagaagatgactcgtgttgaagtgtgctccccagtgctggaggtgaggtttggtagaagatgactcgtgttgaagtgtgctccccagtgctggaggtgaggtttggtagaagatgactcgtgttgaagtgtgctccccagtgctggaggtgaggtttggtagaagatgactcgtgttgaagtgtgctccccagtgctggaggtgaggtttggtagaagatgactcgtgttgaagtgtgctccccaatgctggaggtgaggtttggtagaagatgactcgtgttgaagtgtgctccccaatgctggaggtgaggtttggtagaagatgactcgtgttgaagtgtgctccccagtgctggaggagaggtttggtagaagatgactcgtgttgaagtgtgctccccaatgctggaggtgaggtttggtagaagatgactcgtgttgaagtgtgctccccaatgctggaggtgaggtttggtagaagatgactcgtgttgaagtgtgctccccaatgctggaggtgaggtttggtagaagatgactcgtgttgaagtgtgctccccaatgctggaggtgaggtttggtagaagatgactcgtgttgaagtgtgctccccaatgctggaggtgaggtttggtagaagatgactcgtgttgaagtgtgctccccaatgctggaggtgaggtttggtagaagatgactcgtgttgaagtgtgctccccaatgctggaggtgaggtttggtagaagatgactcgtgttgaagtgtgctccccaatgctggaggtgaggtttggtagatgactcgtgttgaagtgtgctccccaatgctggaggtgaggtttggtagaagatgactacatcaggggggtggatccctcatgaatggtttagcaccacccccttggtgataaccgagctctcgctcagttagttcacaagagatctgcttgtttaaaaagggcatgggacctcctccttcctcttgctcctattgtcaccacgtgacatgcctgtttcccctccgacttctgccattaatataaacttcctcaggactcaccagaagctgaggagaagctgatgccatgcttcctgtacagcctgaagaaccgtgagccaactaaaccctactcttttctttacaaattacccagtctcaggtatttctttacagcaatgcaagaacgaactagcacaacatcagtaagaaaaaaagggtgaaagctacctgttactacgagaacatttgactagaagtcaagaaactaggattcgagttcctgctccagcaataatcagctatggttttagtttcctaagttgtacttgagtgactggaaggagagctaaactatataccaaagaagatgaatggctagttgtaacaggggtaaggtggacttttattctgaatttctaggatccagtgctagctccaacattagaggatattaagactaatgcaatatgcaatgtgttaaccaccaagcaaactaacttagaaatatatgtggtttttataagcaaagcctatgtctatttgatttaaattggctgtacattagcagtgctaaaaaaggcataccaaatgcatcacatgatcacttctttatctgacagagcacaggcatcctatctggtcaagaaaacaaaacaaatcttattgttccatgatttcccacaacataacctttatttctataaagttaagatattataggtgatgtcatctataatagtatctttttagaggtttacattcagaatatgttttcatttcattttattctgaaaactcttcataaggtcaaaagggcatgtcatatccatcttaaagttcagggcagagtttgcccaaggtaaggtcaaaatttagaaatcaaggattctggatctctaggtcacagtgctttctattatcctttatgcatgcactcactaagttgtattctttcattatacaaacatgtataaggaactgggttgggtacttcttgtgaggacccagtttttagttttgacttcgtcattctccaccaataatcactttgtaatcctaagcaagtcacaacttctctgaattttagtttcttggtctgtaaaattgaaacaataatatatgttctttcttgtcattgaaaattagtggggagactgaattagatagaaagctaagagctgaaaagaattctataggtctaatttaatactctccttttacaaatatacaatagagaagctaaattatttgctcaaggtcatgtcattttttttttcctttaagagttgaggactctctatattgcccaggctaaactggaattcctgggctcaagagatcctcctacctctgcctcctgagtaggtggggctcaggagacgcacattaatttttagtaacttggattagaatccaagtcacttaacacctagtgaaattggtcctttcattacactttgcagcgtctaaactaggtgaaaaatttttgaaagctacaacccacctagccttacccaactaaggttttatgaccaaattcagcctttatgccacaaggcatatattgtatgtgaagaattaacatcttccaggaattcagagtaggactaggttacatagcatccttgagagaatggagaaaatagtcactcaaatagttttctgtgtttttggatgagaaaggcagcatgactatattaatgggacattattaccaggaatggctctagccttcaagacctgaaatttaaaatttgtattcaactgtaaaggctagccttcattacacatcatctcctcaagaagctctttactaaattcgtagattctcatgtctatgtgttactaatagctgttgttatatcttgtattgcaattatttctttgttttgtattcttttatttttctttttttttgagacagagtcttgctctgtcacccaggctggagtgcagtggcatgatctccgtcactgcaacctctgcttcccaggttcaagtgattctcctgccttagccttctgagtagctgggattacaggtgtgagccaccacacctggataacattttgtatttttagtagagacagagtttcaccatgttggccaggctggtttcaaactcctgacctcaagaggtctgcccgccttggcctcccaaagtgctaggattacatgcatgagccaccacacatggccttgtttttcattcttaattgaattgcacgtatatgtcttaaaaggcaaactaagtactgtggaagtacagaaaaggaactgatgagattaaggagactccatagaaaagacgtgatatgattctaagagttggagatgaagaagcaacaagagagttcggaaaaaaagataaggaagaagggacaactcaagtgtatgcacagataaatatgcttattcaagttttaaatcttaccacaataacattttcactcctcttgatttcctttccctacttgtttgaacctgcttcaaccatcattcccatcaacttttactttgctctaatcatcattcatcaccctttaaatctttcctcttctactcccatccaactctataaaagttggttcaactctttgaacttgggtagatgactcacattccggtaaaaagtaacataaagtattaagtgtttacaataatgaaaggaaggtaaatcacttcgaaatgataccgatagacttatggtaatactagtctagaatttaagattttttctccttaggctaaaagatatttttatagaaaataaatatattctgtcaacataaattacaaatgttcatttctcaaattgaaaaaggtttatttgtttctatacctaaatagaaaattttatcataatgagcatcaaaagaatcccaaatgagaaaactgctttttgcataagtgtaaattaagagatggaagtcacatttcaccatggcaaacatttgctgcatgcaaaatgactgaagtaagtttagaataattctttttaaattttaaaatatcaatgtcccatacttttttctttctttcttcaagagacagtgtctcgttatgttgcccaggctggagtgctgtggctattcataggtgtcatcaaagagcaatgaagccccaaactcctatggccttaagtgatcctcttgtctcagcctctagagtagcttcgactacaggagtgtaccactatgcctggctcacactttcttttgaacttaaatcaatttatttctgttttaagtctcacatactctctcatttgatttttttcttttttctttttgcatatcccctgttctcaatgttatgtacaatttcaattatggccttcgaaacttttaatgagagaattctttaaagttttaatcgattcctctttgatcaattttatagtaaaagaacacggtgtcttattgtggaaaaaaataggtcttttaaagaagaatgctattgaaacataaagggaagccttttacagaatatttaatcttatacatgtttcttgtgcaacctgtttcaaaaatcagaaatccttaaacataaagagtgtaaggtgactccttgatctattatacctgagagtgaaaaaacccaacctgcggaggttgcagtgagccgagatcgcgccactgcactccagcctggatgacagagcaagactccgtctcaaaaacacaaaaacaaaaacaaaaacaaaaacccaacccagcagcttaagcagtgaaagtattcaaagaatcagaatcagatgtcattaaattttaaaaaccgatgcattacataatgaaattcactatatttcattctttttatttatttacttatttatttgagaacaggcctctctctgtcactcaggatggagtaaaatggtacaatcatagctcaatgcagcctcaatctcccgggcccaagtgatccacccacctcagcctccctagtaaccaggactacaggtgcccaccaccacacttggctaatttttggaaacagggtgtcactatgttgcctaggctggtcttgaaatcctggtctcaagtgatcctctcatctcagcctcccaaagtgttgggattataggtgtgaaccacagatcctggccaagattcactatattttgaattggttgtatatgtaggctatattctttcctttttgttttttttttagacaaggtcttgatctgtcaccaggctgaagtgcagtggtacaatcaaagctcactgcagtctcaacctcctaggctcaaacaatcctcccacttcagcctcccaaccagctgggaccacaggttgacatcaccacacccaggtaatttttaaattttttatggagacagggtttccctatgttgccaaagctgatacactatatttcaaatcagtctacaaggtcattatggcaacagtcctaaaaaaaggcttgtcttcaccattttttaaaagtattatactttaagtattgggatacatgtgcagaacgtgcaggtttgttacataggtatacacgtgccatggtggtttgctgcacccatcaacccatcatctatgttaagtatttctcttaatgctatccctcctctaacccccaaccccgacaggccccgatgtgtgatgttcccctccctgtgtccatgtgttctcattgttcagttcccacttatgagtgagaacatgcggtgtttggttttctgttcctgtgttagtttgctgaaagtgatggtttccagcttcatccatgtccctgcaaaggacatgaactcatccttgttcatggctgcatagtattcgatggtatatatgtgccacattttctttatcctgtctatcattgatgcgcatttgggtgggttccaagtctttgctattgtaaatactgctgcaataagcatatgtgtgcatgtgtctttatattagaattatttataatcctttgggtatatacccagtaatgggcttgctgggtcaaatggtatttctggttctagatccttgaggaatcgccacactgtcttccacaatggttgaactaatttacactcccaccaacagtgtaaaagcattcctattctccacatcctctccagcatctgttgtttcctgacgttttaataatcgccattctaacggccatgagatggtatctcattgtggttttgatttgcatttcattaatgaccgctgatgatgagcttttgttcatatgcttgttggccacataaatgtcttcttttgagaagtgtcagttcatatccttcacccactttttgatggagttgtttgcttttttcctgtaaatttgtttaagttccttgtagattctggactttagccctttgtcagatggatagatcgcaaaaattttctcccattctgtaggttgcctgttcactctgatgatagtttcttttgctgtgcagaagctctttagtttaattagatcccatttgtcaattttggcttttgttaccattgcttttggtgttttggtcaggaagtctttgcccatgcctatgtcctgaatggtattgcctaggttttcttctagggttttcatggctttaggtcttacatttaactctttaatccatcttgagttaatttttgtataaggtgtaagaaaggggtccagtttcagttttctgcacatggctagccagttttcccaacaccatttattaaacggaatcctttgtctcatctttgtcaaagatgagatggttgtagatgtgtggcattatttctgaggcctctcttctgttccattggtcaatatatctgttttggtaccaataccatgctgttttggttattgtagccttgtagtatagtttgaagtcaggtagcatgatgcctccagctttcttctttttgcttaggattgtcttggctatatgggcttttttggttccatatgaaatttaaagtagttttttctaattttataaaggaagtcaatggtagctcgatagggatagcattgaatctataaattactttgagtggtatggccattttcacaagattgattcttcctatccatgaacatggaatgttcttccatttgtttgtgtcctctttatttccttgagcagtgaagtttgtagttcttgaagatgtccttcacatcccttgtgtgttgtatgcctaggtattttattctctttgtagcatttgtgaatgggagttcactcatgatttggctctctgtttgtctattattgttgtataggaatacttgtgatttctgcacattaattttgtatcctgagactttgctgaagttgcttatcagcttaaggagattttgggctgagataatggggttttctaaatagacaatcattgtctctgcaaacagagacaatttaatttcctctcttcctacttgaatacgctttatttcttcctcttttctaactgccctgaacagaacttccaatactatgttgaataggagtggtgagagagggcatccttgtcttgtgccagttttcaaagggaatgcttccagcttttgctgattcagtatgatattggctgtgggtctgccataaatagctcttattattttgagatacattccatcaatatgaagtttattgagagtttttagcatgaagcagtgttgaatttcatcaatggccttttctgcatctattgacataatcatgtggtttttatctttggttctgtttatgtgatggattacgtttattgatttgcatatgttgaaccagccttgcatcccagggatgaagccgacttgtttgtggtggataagctttttgatgtgctatggattcagtttgccagtattttattgagggttttcacgtcgatattcatcaaggatattggccgaaattttctttttttgttgtgtctctggcaggttatggtatcaggatgattcttggcttataaaatgagttaaggaggactctcttttcttctattgcttggaatagtttcagaaggaatagtaccagctcctccttgtacctctggcagaattcagctgtgaatccatctgctcctgggatttttttggttggtacgctattaattactgcctcaatttcagaacttgttattggtctattcagggattcgacttcttcctggtttactcttgggagggtgtatgtgtccaggaatttatccatttcttctagattttctagattatttgcatagagatgtttacagtattctctgatggtagtttgtatttctgtgggatcagtggtgatatcccctttagcatttttttattgtgtctatttgattcttctctcttttcttctttattagcctggctagtggtctatctattttgttaatcttttcaaaaaaccagctcctggattcattgattttttgaagggtttttcgtgtttctctctccttcagttcttctctgaccttggttatttcttatcttctgctagcttttgaattagtttgctcttgcttctctagttcttttaattgtgatgttagggtgtcaattttacatcttttctgctttctcctgtgggcatttagtgctataaacttccctctaaacattgctttagctgtgtcccagagatttgggtatgttgtgtcttggttctcattggtttcaaagaacttctttgtttctgccttaatttcattattttcccagtattcattcaggaacaggttgttcagtttccatgcagttgtgcaggtttgagtgagtttcttaatcctgagttctaatttgattgcactgaggtctcagagactgtgttatgatttccattgttttgcatttgttgaggagtgttttactcccaatcatgtggttagttttagaataagtgtgatgtggtgctaagaagaatgtctattctgttgatttggggtggagagttctgtagatgtctattaggtctgcttggtgcagagctgagttcaagtcctgaatatccttgttaattttctgtctcattgatctgtcaaatattgacagtggggtgttaaagtctcccactattattgtgtgggagtctaagtctctttgtaggtctccaaggacttgctttatgaatctgggtgctcctgtattgggtgcatacatacttaggatagttagctcttcttgttgcattgatccctttaccattatgtaatgcccttcttgtcttttttgatcttcgttggtttaaagtctgttttatcagagactaggattgcaacccctgcttttttttttttttgctttccatttgcttggtaaatcttcctccattcccttatttagagcacgtgtctttgcatgtgagatgagtctcctgaatacagcacactgatgggtcttgactctttatccaacttgccagtctgtgtcttttaattgtggcacttagcccgtttacttttaaggttaatattgttatatgtgaatctgatcctgtcattatgatgctagccagttagtttgcccattagttgatgcccattagtttcttcatagtgtcaatggtctttacaatttggtatgtttttgcagtggctgggaccaatttttcctttccatatttagtgcttccttcaggagctcttgtaatgcaggcctggtggtgacaaaatctctcaacatttgcttatctgtgaaggattttatttctcattcacttatgaagcttagtttggctggatacaaaattctgggttgaaaattcctttctttaagaatgttgaatattggcccccactcttttctggcctgtagggtttctgcagagagatccactattagtctgatgggcttccctttgtaggtaacccgacctttctctctggctgcttttaacattttttccttcatttcaaccttggtgaatctgacgattatgtgtcttggggttgctcttcttgaggagtatctttgtggtgttctctgtatttcctgaattggaatgttggcctgtcgtgctaggttggggaaattctcctggataatatcctgaagagtgttttccaacttggttcaattctccctgtcactttcaggtacagggacaatcaaacataggtttggtcttttcacatagtcccctatttcttggaggctttgttcgtttcttttcattctttttcctctaatcttgtcttcacactttatttcataaaggtgatcttcaatctctgatatcctttcttctgctttggctgttgatacttgtgtatgcttcacgaagttcttgtgctgtgtttttcagctccatcaggtcatttatgttcttctctaaactgcttattttaggtagcaattcctctaaccttttttcaaggtttttagcttccttgcattgggttagaacatggtcctttggcttggaggagtttattattctctgtccagttttgttcccttgctggcgaggagttgtgattctttggaagagaaaaggcattctggtttttggaattgtcagcctttttgcgctggtttttcctcatcttcatggatttatctacctttggtcttcgatgttggtgaccttcaaatggggtttctgtgtggaagtcctttttgttgatattgatgctattcatttctgtttgttagttctccttctaacagtcaggcccctctgctgcaggtctgctggaatttgctggaggtccactccagaccctgtttgtctggatatcaccagcagaggttgcagaacaacaaagattgctgcctgttccttcctctggaagctttgtcccagaggggcacccaccagacggcagctggagctctcctgtgtgagttgtctgtcgattcctgctgggaggtgtttcccagtcaggagacatgggggtcagagacccacttgaggaggcagtctgtcccttagcagagctcgagcaatgtcctgggagatctgctgctctcttcagagccagcaggcaggaacgtttaagtctgctgaagctgcaccaatagccacccatagcacccttcccccaggtgctctgtcccagggagatggcagttttatctataagcccctgactggggctgctgcctttttttcagagatgccctgcccagagaggaggactctagagaagcagttgggctacagtgggctccacccagttcaaacttcccggtggctttgcttacactgtgaggtgaaaaccacctactcaagcctcagtaatggcagaagcccctcctcccaccaagctccagcatcccaggtcgacttcagactcctgtgctggcagtaagagtttcaagccagtggatcttagcttgctgggctctgtgggggtgggatccactgagctagaccacttggctccctggcttcagcccctgttccaggggagttaacagttctgtctcactggtgtttcaggtgccactggagtacgaaaaagaaaaaatcctgcagctagctcggtgtctgcccaaacggctgcccagttttgtgcttgaaacccagggcctgggtggtgtaggcacccgagggaattgcctggtctaagggttgtgaagatcacggaaaaggcatagagtctgggccggaatgcatcgttcctcgtggcacattccctcatggcttcccttagctaggggaggaagttccctggccccttgcacttcccgtgtaaggcgatgccccaccctgctttggctcgccttccatgggctgcaccccctgtctgaccagtctcaatgagatgagctgggtacctcagttggaaatagagaaatcacccaccttctgcgttgatttcgctgggagctgcagactccagctgttcctgtgttcaccattttaacatctatctggctctgtttaattttcgctccccccaaaaaatccatataagaaataagttgattctcagagttaagttccagaagtgtagagatctcacattaaaataacagtttgcctgagtttatcaaatacaaaagtatctattaaatataattgattatatggattcattacctccctttgcaagtagttactctgtatttttccaattctaacctaagaatttttcaaagtttctaaattcccctttggttccttaattagaggccaaatttaatatctggaaaaaatgcagtattcaattatcttcttcctgttcaaacagtccctctgctatggctcttcctcagtagaaacaactggcaacaaaattcaagtttatgattcattcatcagcaaacatgtgagaatcatctacaaagaaccaagaattgtggtaagaattatacaaacatttactatgatactgataataatgaaaagcctaattcaacaaatagtgatctatccatttttaagccccagtaactcaaacattaaaaaactataaatgaacaggccgggcacggtggctcacacatggaatcccagcacttggggaggctgaagtgggtggatgacttgaggtcaggagttcaagaccagcctggccacatggtaaaaccctatctctactaaaaatacaaaaattagctgggtgtggtggcgggtgcccataatcccagctactcgggaggctgaggtgggaaaattgctggaacctgggaggcagaggttgcagtaagacaagatcgcaccactgcactccagcctgggcggcagagtgagaaaaaaaaaagaaagaaagaaaaagaaaaagaaaagaaaagaaaaaactatagatgaatgacttaagtgaataacaagtcaagtgagtaatgacaatatgtagaaaaatattccaagtagaattaataaattttaattatttgaatgagaaaatgccgccatattcaaaatgaaagtaaaagtaggatgagatcagcatatacaactgcatatatttcattaattaaagatgactaaagaggagactatcaatttgaaagaagattactaagattttgtttttttaaaacatactgaaccaaataaaaaaaaaataacagtaagacataactactaacagaaaattttttccaatgagaatacaagcttattaaccactccttttttcattaatgctcactcagcttctaccagccttctaataagaacacacacatacatcttttctataccaaatgtgatataaaagatattcaacccttcaaaattttaagtattggttataagtaatactcatctttttttgtcatttgataatagatgaattttctagtccagacagtagaaatagattatagttcatcaacaaatgacaaaaattgctgagtgtgaaagaatggtgcaaaacaacacaaaacattttactgtaataagttatttttaagctagctaagtaatttatgagaaaaaaataaattgttcttaaattacttaatacccaagaggaagagtttcagcttaaactagaccaaattaaaaaaataataacagtaaggcataactagatctagccttttctgaagaaaatgatatttaaaacaagaaaaacatgtgccatagaggttgtgatatcctcaatctttgaggaaataacacctacatttaagtaccctcaccttacacacactttcttcttctgattttggagtctttaaaatataagtatactttttttttttttttttgagatagtctcgctgtcgcccaggctggagtgcagtggcttgatcttggttcactgcaacatctgcctcccaggttcaagcgattctcctgcctcagtctcctgggtagctgggactacaggcacgcactattgccctaggctagttttgtatttttggtagagatggggtttcgccattttggccaggctgctctcaaactcctgatctcaggtgatccacctgcctcggccttccaaagtgctgggattataggcgtgagccactgcacctggcccaaaatatgagtatactttaaatataaacatataaaccattcatagaaacttttagatggagacaaaatctgaaatatatttcaatatatagttaatgtcatattaaggggttaagggaagagctgggaggaagaaggaagataagactatgaacctaaaattttgccaagtagtaggatttttattctctaaaccttatttcaatcaatttaaggttaaaaaaattttgcttaaaaagatgcaatttcattttgtgcttagaaagtaaaattaacaccacaccaaagataaacattcttgagtttgttcttttggagaaatgagtaatttaaaaatttaaacagctctaaaatattaatacaactgcataggaaagaggaaagaatgttagaaaattgcttatcaaaaactaatgagacccaggataagagctactcataatgataacaattaaatattgagtatttttcagaaaccaagagtcaaatgcttgttataccttaaaggttaaaggcccctgtttcatagtcattaaacagttctttgaaatgctatgttaagatatcactaacaatttcaatttataaatacacacccatacacattttcttttctaaattgtcatatcccaaacacgaaaacaatcgaataagagaacaggctgaatagtgatttttttctttcaattattactactgaaacaaatcaaaacaactctgagaggttgccagcaccgcagaaggcatttccacataaaaagagttaatgagcagaccacgcagttcaacagccagtgaccatttgcaagcctcctaggtatattcccagcagacacattatcagattaaaggaagtgcaaacaaactgggctgaaagtttctttgtccaaacccagcctcttcttcctgtgatgtcatattacaaatctaggaagcctttctttttcacttcagagaaagcgacctcaagatacaactggcaactgaggaaaaggcctcaattcaacaagagctaacaagcttgggagtttatttcggaatctttaaaagactcttctgcttacccacaatctgggatccactgcaggaaaacaaaaaaggaaaacttcatttaaaagaagcaagaagtaaaatgggacaaattgggaatgtttaagtctctgaaactctgcactgaaaagaaaataagattgataacttaagcttaacattctgaggcataaagaaacattaactttggagtattcatcttgactactgaaatacaagtttagaagacaagtggtttcattctggtcacagatcacagcttttctttaaatttataatcctatgggttggactcgttgactgtattttttaaaggttgctcgtcagttaactgagccttggaattcatggattttctaaagactaacaaatgaaaatattttcctgttgaagaacccagcggaaattttacagcaacaaatttcatgtttcttttgggtatttctgagaaaaaggaaatatttataaaaccatccaaagatccagataatttgcaaataaattggaggttatagaggttataatctgaatcccaaaggagactgcagctgatgaaagtgcttccaaactgaaaattggacgtgcctttacgatggtaagcgttaacagctcccactgcttctataatgactcctttaagtacactttgtatgggtgcatgttcagcatggtgtttgtgcttgggttaatatccaattgtgttgccatatacattttcatctgcgtcctcaaagtccgaaatgaaactacaacttacatgattaacttggcaatgtcagacttgctttttgtttttactttacccttcaggattttttacttcacaacacggaattggccatttggagatttactttgtaagatttctgtgatgctgttttataccaacatgtacggaagcattctgttcttaacctgtattagtgtagatcgatttctggcaattgtctacccatttaagtcaaagactctaagaaccaaaagaaatgcaaagattgtttgcactggcgtgtggttaactgtgatcggaggaagtgcacccgccgtttttgttcagtctacccactctcagggtaacaatgcctcagaagcctgctttgaaaattttccagaagccacatggaaaacatatctctcaaggattgtaattttcatcgaaatagtgggattttttattcctctaattttaaatgtaacttgttctagtatggtgctaaaaactttaaccaaacctgttacattaagtagaagcaaaataaacaaaactaaggttttaaaaatgatttttgtacatttgatcatattctgtttctgttttgttccttacaatatcaatcttattttatattctcttgtgagaacacaaacatttgttaattgctcagtagtggcagcagtaaggacaatgtacccaatcactctctgtattgctgtttccaactgttgttttgaccctatagtttactactttacatcggacacaattcagaattcaataaaaatgaaaaactggtctgtcaggagaagtgacttcagattctctgaagttcatggtgcagagaattttattcagcataacctacagaccttaaaaagtaagatatttgacaatgaatctgctgcctgaaataaaaccattaggactcactgggacagaactttcaagttccttcaactgtgaaaagtgtctttttggacaaactatttttccacctccaaaagaaattaacacatggacattttaaagtctttagtataaagaaaatttgtattcaatgtgttaagcattaacatgtattttatttgtgtatccactccatctgatttttctgagccattttgatttgttccttcattaaaaaaaatctcttaaagttatttagtgtctaaaagtgactgacttaaattatgtggtgccaatctgtaatgtctttgaattcctttttatattaaatgatttaatttactaaattattttatactaatttatgtttatgtaatattttattcaacataccatttttattttgaattaaaactactaatcatatttcttaaactatgattaacaaatgagaaagaagagtaatttgaaataaatgttttgggagtattttaaaatatatgtataactaaaaacgtagtcttctgtagaaatgatgacatattaacattgttggaagatcttgagcagtaacacattgataacattttgggagtttggtctgtgtaggcaatttgtagaaagcagatgctatctatatggtatgctttgaagttactattttgattttctgtatataatgcacccaatttcaaaatgatatatgccacaaaaattgagagggagaaaactaataaaatgtcattttgctaagtttttacttagcaaaaaaataaaatactaccagaggtacattttaaaggtgcacttacatcacagtcaagaacttatgttttaagtatgttaggtcggagaaacatcagagatagtgaaatatagtacttggcatttagaaagtaatatactgtcacgtgttgcttatgatgcttgcttaggtaattatgtcgtgcaaacatcacagtgtgtacttacacaactctagaaggtatacactattacacacctaggcttatatggtacaccctactgcttctaggctacaaacctatacagcatgctactgcaaagaatactgtaggcaactgtaaaataatggtacttgtgtatctaaatacatctcaacacagaaaaggtacagtaaaaatatagtatgataatcttatgagaccaccatcgtttatgcggtcattgaccaaaatgctattacgtgatgcatgactgtaaatattttcttttccttcactcttctggccattttaagccaaatattgggaagttcaaaacattatttcaagtaggcaagatgttattgtacgtcaaaatgattaattaaggacacagttcct

SEQ ID NO: 5 - DNA, 465 bp, homo sapiens
atggtaagcgttaacagctcccactgcttctataatgactcctttaagtacactttgtatgggtgcatgttcagcatggtgtttgtgcttgggttaatatccaattgtgttgccatatacattttcatctgcgtcctcaaagtccgaaatgaaactacaacttacatgattaacttggcaatgtcagacttgctttttgtttttactttacccttcaggattttttacttcacaacacggaattggccatttggagatttactttgtaagatttctgtgatgctgttttataccaacatgtacggaagcattctgttcttaacctgtattagtgtagatcgatttctggcaattgtctacccatttaagtcaaagactctaagaaccaaaagaaatgcaaagattgtttgcactggcgtgtggttaactgtgatcggaggaagtgcacccgccgtttttgtttag

SEQ ID NO: 6 - protein, 154 aa, homo sapiens
MVSVNSSHCFYNDSFKYTLYGCMFSMVFVLGLISNCVAIYIFICVLKVRNETTTYMINLAMSDLLFVFTLPFRIFYFTTRNWPFGDLLCKISVMLFYTNMYGSILFLTCISVDRFLAIVYPFKSKTLRTKRNAKIVCTGVWLTVIGGSAPAVFV

SEQ ID NO: 7 - DNA, 483 bp, homo sapiens
atggtaagcgttaacagctcccactgcttctataatgactcctttaagtacactttgtatgggtgcatgttcagcatggtgtttgtgcttgggttaatatccaattgtgttgccatatacattttcatctgcgtcctcaaagtccgaaatgaaactacaacttacatgattaacttggcaatgtcagacttgctttttgtttttactttacccttcaggattttttacttcacaacacggaattggccatttggagatttactttgtaagatttctgtgatgctgttttataccaacatgtacggaagcattctgttcttaacctgtattagtgtagatcgatttctggcaattgtctacccatttaagtcagactctaagaaccaaaagaaatgcaaagattgtttgcactggcgtgtggttaactgtgatcggaggaagtgcacccgccgtttttgttcagtctacccactctcagggtaa

SEQ ID NO: 8 - protein, 160 aa, homo sapiens
MVSVNSSHCFYNDSFKYTLYGCMFSMVFVLGLISNCVAIYIFICVLKVRNETTTYMINLAMSDLLFVFTLPFRIFYFTTRNWPFGDLLCKISVMLFYTNMYGSILFLTCISVDRFLAIVYPFKSDSKNQKKCKDCLHWRVVNCDRRKCTRRFCSVYPLSG

SEQ ID NO: 9 - DNA, 20 bp, Artificial sequence
  source: /note=“Description of artificial sequence Primer”
agcagtgggagctgttaacg

SEQ ID NO: 10 - DNA, 374 bp, Artificial sequence
  source: /note=“Description of artificial sequence DNA-Probe”
ttataccaacatgtacggaagcattctgttcttaacctgtattagtgtagatcgatttctggcaattgtctacccatttaagtcaaagactctaagaaccaaaagaaatgcaaagattgtttgcactggcgtgtggttaactgtgatcggaggaagtgcacccgccgtttttgttcagtctacccactctcagggtaacaatgcctcagaagcctgctttgaaaattttccagaagccacatggaaaacatatctctcaaggattgtaattttcatcgaaatagtgggattttttattcctctaattttaaatgtaacttgttctagtatggtgctaaaaactttaaccaaacctgttacattaagtagaagca

SEQ ID NO: 11 - DNA, 23 bp, Artificial sequence
  source: /note=“Description of artificial sequence Primer”
tggaggttatagaggttataatc

SEQ ID NO: 12 - DNA, 22 bp, Artificial sequence
  source: /note=“Description of artificial sequence Primer”
ttataccaacatgtacggaagc

SEQ ID NO: 13 - DNA, 22 bp, Artificial sequence
  source: /note=“Description of artificial sequence Primer”
tgttaatttcttttggaggtgg

SEQ ID NO: 14 - DNA, 22 bp, Artificial sequence
  source: /note=“Description of artificial sequence Primer”
ttgtgtgtctttaagaccctcc

SEQ ID NO: 15 - DNA, 20 bp, Artificial sequence
  source: /note=“Description of artificial sequence Primer”
tcctgcctgggccgcagagc

SEQ ID NO: 16 - DNA, 23 bp, Artificial sequence
  source: /note=“Description of artificial sequence Primer”
aaatgataccgatagacttatgg

SEQ ID NO: 17 - DNA, 24 bp, Artificial sequence
  source: /note=“Description of artificial sequence Primer”
tctttgatgacacctatgaatagc

SEQ ID NO: 18 - DNA, 23 bp, Artificial sequence
  source: /note=“Description of artificial sequence Primer”
ttataggtgtgaaccacagatcc

SEQ ID NO: 19 - DNA, 22 bp, Artificial sequence
  source: /note=“Description of artificial sequence Primer”
tgttgccataatgaccttgtag

SEQ ID NO: 20 - DNA, 22 bp, Artificial sequence
  source: /note=“Description of artificial sequence Primer”
tggggtttctgtgtggaagtcc

SEQ ID NO: 21 - DNA, 24 bp, Artificial sequence
  source: /note=“Description of artificial sequence Primer”
tctctatttccaactgaggtaccc

SEQ ID NO: 22 - DNA, 23 bp, Artificial sequence
  source: /note=“Description of artificial sequence Primer”
aatggatagatcactatttgttg

SEQ ID NO: 23 - DNA, 21 bp, Artificial sequence
  source: /note=“Description of artificial sequence Primer”
aggaagtgcaaacaaactggg

SEQ ID NO: 24 - DNA, 20 bp, Artificial sequence
  source: /note=“Description of artificial sequence Primer”
agcagtgggagctgttaacg

SEQ ID NO: 25 - DNA, 23 bp, Artificial sequence
  source: /note=“Description of artificial sequence Primer”
aaaggaattcaaagacattacag

SEQ ID NO: 26 - DNA, 20 bp, Artificial sequence
  source: /note=“Description of artificial sequence Primer”
cctaccattgacactctcag

SEQ ID NO: 27 - DNA, 16 bp, Artificial sequence
  source: /note=“Description of artificial sequence Primer”
tagggccatccattct

SEQ ID NO: 28 - DNA, 20 bp, Artificial sequence
  source: /note=“Description of artificial sequence Primer”
acctgttgtatggcagcagt

SEQ ID NO: 29 - DNA, 20 bp, Artificial sequence
  source: /note=“Description of artificial sequence Primer”
ggttgactctttccccaact

SEQ ID NO: 30 - DNA, 16 bp, Artificial sequence
  source: /note=“Description of artificial sequence Primer”
tgcaggtgggagtcaa

SEQ ID NO: 31 - DNA, 24 bp, Artificial sequence
  source: /note=“Description of artificial sequence Primer”
aaataacaagaagtgaccttccta

SEQ ID NO: 32 - DNA, 16 bp, Artificial sequence
  source: /note=“Description of artificial sequence Primer”
ccaacatcgggaactg

SEQ ID NO: 33 - DNA, 21 bp, Artificial sequence
  source: /note=“Description of artificial sequence Primer”
tgcattctttaagtccatgtc

SEQ ID NO: 34 - DNA, 19 bp, Artificial sequence
  source: /note=“Description of artificial sequence Primer”
ttgaggacctgtcgttacg

SEQ ID NO: 35 - DNA, 21 bp, Artificial sequence
  source: /note=“Description of artificial sequence Primer”
ttatagagcagttaaggcaca

SEQ ID NO: 36 - DNA, 19 bp, Artificial sequence
  source: /note=“Description of artificial sequence Primer”
cctgcatttgtgtacgtgt

SEQ ID NO: 37 - DNA, 23 bp, Artificial sequence
  source: /note=“Description of artificial sequence Primer”
cagagccgtggtagtatattttt

SEQ ID NO: 38 - DNA, 20 bp, Artificial sequence
  source: /note=“Description of artificial sequence Primer”
aggactaaagatgaatgctc

SEQ ID NO: 39 - DNA, 20 bp, Artificial sequence
  source: /note=“Description of artificial sequence Primer”
gacatgactccatgtttggt

SEQ ID NO: 40 - DNA, 25 bp, Artificial sequence
  source: /note=“Description of artificial sequence Primer”
cctggcctgttagtttttattgtta

SEQ ID NO: 41 - DNA, 22 bp, Artificial sequence
  source: /note=“Description of artificial sequence Primer”
cccagtcttgggtatgttttta

SEQ ID NO: 42 - DNA, 20 bp, Artificial sequence
  source: /note=“Description of artificial sequence Primer”
cttggaaaccaacaagtcac

SEQ ID NO: 43 - DNA, 19 bp, Artificial sequence
  source: /note=“Description of artificial sequence Primer”
attttgccacctagaacgg

SEQ ID NO: 44 - DNA, 21 bp, Artificial sequence
  source: /note=“Description of artificial sequence Primer”
acagtttttcgaggccatatc

SEQ ID NO: 45 - DNA, 25 bp, Artificial sequence
  source: /note=“Description of artificial sequence Primer”
ggttaaataaatctccatccagaag

SEQ ID NO: 46 - DNA, 20 bp, Artificial sequence
  source: /note=“Description of artificial sequence Primer”
tccagataggcagattcaat

SEQ ID NO: 47 - DNA, 20 bp, Artificial sequence
  source: /note=“Description of artificial sequence Primer”
ccttcttcttcccattgata

SEQ ID NO: 48 - DNA, 16 bp, Artificial sequence
  source: /note=“Description of artificial sequence Primer”
tgcagaaatgtgagcc

SEQ ID NO: 49 - DNA, 19 bp, Artificial sequence
  source: /note=“Description of artificial sequence Primer”
tccacctagagcaactacc

SEQ ID NO: 50 - DNA, 22 bp, Artificial sequence
  source: /note=“Description of artificial sequence Primer”
tggtttgttgcagaaagcacac

SEQ ID NO: 51 - DNA, 22 bp, Artificial sequence
  source: /note=“Description of artificial sequence Primer”
ttgggccttgtcaaccttcata

SEQ ID NO: 52 - DNA, 20 bp, Artificial sequence
  source: /note=“Description of artificial sequence Primer”
ggcaaagccttgctcttaat

SEQ ID NO: 53 - DNA, 20 bp, Artificial sequence
  source: /note=“Description of artificial sequence Primer”
ggctgtgctcttccaaaata

SEQ ID NO: 54 - DNA, 21 bp, Artificial sequence
  source: /note=“Description of artificial sequence Primer”
agggatcttcagagaaacagg

SEQ ID NO: 55 - DNA, 21 bp, Artificial sequence
  source: /note=“Description of artificial sequence Primer”
tgacactatcagctctctggc

SEQ ID NO: 56 - DNA, 20 bp, Artificial sequence
  source: /note=“Description of artificial sequence Primer”
attagcccaggtatggtgac

SEQ ID NO: 57 - DNA, 25 bp, Artificial sequence
  source: /note=“Description of artificial sequence Primer”
gctgtggtatgagttacttaaacac

SEQ ID NO: 58 - DNA, 228 bp, homo sapiens
  variation 1: /replace=“c” /replace=“g” /replace=“t”
aatttttttttttttttcacagcaacaaatttcatgtttcttttgggtatttctgagaaaaaggaaatatttataaaaccatccaaagatccagataatttgcaaataaattggaggttatagaggttataatctgaatcccaaaggagactgcagctgatgaaagtgcttccaaactgaaaattggacgtgcctttacgatggtaagcgttaacagctcccactgct

SEQ ID NO: 59 - DNA, 20 bp, artificial feature
  source: /note=“Description of artificial sequence Primer”
agcagtgggagctgttaacg

SEQ ID NO: 60 - DNA, 21 bp, Artificial sequence
  source: /note=“Description of artificial sequence Primer”
gaaccagtgagtttttattac

SEQ ID NO: 61 - DNA, 21 bp, Artificial sequence
  source: /note=“Description of artificial sequence Primer”
agacacagcatataatacatg

SEQ ID NO: 62 - DNA, 20 bp, Artificial sequence
  source: /note=“Description of artificial sequence Primer”
agccacatcgctcagacacc

SEQ ID NO: 63 - DNA, 21 bp, Artificial sequence
  source: /note=“Description of artificial sequence Primer”
tcctcttgtgctcttgctggg

SEQ ID NO: 64 - DNA, 20 bp, Artificial sequence
  source: /note=“Description of artificial sequence Primer”
tcttcccagaatatatggga

SEQ ID NO: 65 - DNA, 25 bp, Artificial sequence
  source: /note=“Description of artificial sequence Primer”
agctgtaaaagtgtttgtttgatgt

SEQ ID NO: 66 - DNA, 20 bp, Artificial sequence
  source: /note=“Description of artificial sequence Primer”
agcattgtttcatgttggtg

SEQ ID NO: 67 - DNA, 20 bp, Artificial sequence
  source: /note=“Description of artificial sequence Primer”
cagcagtgaaggtctaagcc

SEQ ID NO: 68 - DNA, 17 bp, Artificial sequence
  source: /note=“Description of artificial sequence Primer”
gtttcgccaagcctgtt

SEQ ID NO: 69 - DNA, 22 bp, Artificial sequence
  source: /note=“Description of artificial sequence Primer”
gttgacaataaaatacgccaca

SEQ ID NO: 70 - DNA, 16 bp, Artificial sequence
  source: /note=“Description of artificial sequence Primer”
gatggcaccattgcac

SEQ ID NO: 71 - DNA, 18 bp, Artificial sequence
  source: /note=“Description of artificial sequence Primer”
cagcacatccaaacaagg

SEQ ID NO: 72 - DNA, 22 bp, Artificial sequence
  source: /note=“Description of artificial sequence Primer”
ctgtgggattccttagtgatac

SEQ ID NO: 73 - DNA, 23 bp, Artificial sequence
  source: /note=“Description of artificial sequence Primer”
atattcagacaaaagccaagtta

SEQ ID NO: 74 - DNA, 27 bp, Artificial sequence
  source: /note=“Description of artificial sequence Primer”
ctgggaaggctttcttgctcacttcag

SEQ ID NO: 75 - DNA, 27 bp, Artificial sequence
  source: /note=“Description of artificial sequence Primer”
gttggatatcagcccaagcacgaagac

SEQ ID NO: 76 - DNA, 83 bp, Artificial sequence
  source: /note=“Description of artificial sequence Primer”
cgaagcttccgccgccatgggtaagcctatccctaaccctctcctcggtctcgattctacgatggtaagcgttaacagctccc

SEQ ID NO: 77 - DNA, 29 bp, Artificial sequence
  source: /note=“Description of artificial sequence Primer”
gcctcgagtcaggcagcagattcattgtc

SEQ ID NO: 78 - DNA, 29 bp, Artificial sequence
  source: /note=“Description of artificial sequence Primer”
gcctcgagctaaacaaaaacggcgggtgc

SEQ ID NO: 79 - DNA, 29 bp, Artificial sequence
  source: /note=“Description of artificial sequence Primer”
gcctcgagttaccctgagagtgggtagac

SEQ ID NO: 80 - DNA, 20 bp, Artificial sequence
  source: /note=“Description of artificial sequence Primer”
tggattaggagagcttctgg

SEQ ID NO: 81 - DNA, 23 bp, Artificial sequence
  source: /note=“Description of artificial sequence Primer”
tggaagtcacatttcaccatggc

SEQ ID NO: 82 - DNA, 22 bp, Artificial sequence
  source: /note=“Description of artificial sequence Primer”
tctaagggttgtgaagatcacg

SEQ ID NO: 83 - DNA, 23 bp, Artificial sequence
  source: /note=“Description of artificial sequence Primer”
tcttcctcagtagaaacaactgg

SEQ ID NO: 84 - DNA, 22 bp, Artificial sequence
  source: /note=“Description of artificial sequence Primer”
aagatacaactggcaactgagg

SEQ ID NO: 85 - DNA, 20 bp, Artificial sequence
  source: /note=“Description of artificial sequence Primer”
agcagtgggagctgttaacg

SEQ ID NO: 86 - DNA, 23 bp, artificial feature
  source: /note=“Description of artificial sequence Primer”
agcatgacttcgatctgatcagc

SEQ ID NO: 87 - DNA, 22 bp, artificial sequence
  source: /note=“Description of artificial sequence Primer”
tgagatagaagtaggccaaccc

SEQ ID NO: 88 - DNA, 21 bp, artificial sequence
  source: /note=“Description of artificial sequence Primer”
attttcttctacgtgcggcgg

SEQ ID NO: 89 - DNA, 22 bp, artificial sequence
  source: /note=“Description of artificial sequence Primer”
tgtatagtggacagactcgcgg

SEQ ID NO: 90 - DNA, 21 bp, artificial sequence
  source: /note=“Description of artificial sequence Primer”
atctacgtgtacgtcaagagg

SEQ ID NO: 91 - DNA, 21 bp, artificial sequence
  source: /note=“Description of artificial sequence Primer”
ttgtaggagtagatgatgggg

SEQ ID NO: 92 - DNA, 22 bp, artificial sequence
  source: /note=“Description of artificial sequence Primer”
tcgtactattaggactaggagg

SEQ ID NO: 93 - DNA, 22 bp, artificial sequence
  source: /note=“Description of artificial sequence Primer”
acaacagttcagagttgcaagg

SEQ ID NO: 94 - DNA, 22 bp, artificial sequence
  source: /note=“Description of artificial sequence Primer”
tgtactactttagcgccgaggg

SEQ ID NO: 95 - DNA, 22 bp, artificial sequence
  source: /note=“Description of artificial sequence Primer”
agaacgagaggcgttgggagtc

SEQ ID NO: 96 - DNA, 23 bp, artificial sequence
  source: /note=“Description of artificial sequence Primer”
tggaggttatagaggttataatc

SEQ ID NO: 97 - DNA, 22 bp, artificial sequence
  source: /note=“Description of artificial sequence Primer”
tgttaatttcttttggaggtgg